SEQUENCE LISTING (91 sequences)

SEQ ID NO: 1 - DNA, 20 bp, Artificial Sequence
  flgE ncRNA_19_003_1
tttatccaatccatcactgt

SEQ ID NO: 2 - DNA, 20 bp, Artificial Sequence
  flgE ncRNA_19_003_2
tagcattggttctgatggaa

SEQ ID NO: 3 - DNA, 20 bp, Artificial Sequence
  fliR ncRNA_19_003_3
aaacctatgcgatgaacagc

SEQ ID NO: 4 - DNA, 20 bp, Artificial Sequence
  fliR ncRNA_19_003_4
acatttcgatgtccaaatag

SEQ ID NO: 5 - DNA, 20 bp, Artificial Sequence
  flhG ncRNA_19_003_5
tcttctgctgataaaataaa

SEQ ID NO: 6 - DNA, 20 bp, Artificial Sequence
  flhG ncRNA_19_003_6
caaacagcacataatcgaac

SEQ ID NO: 7 - DNA, 20 bp, Artificial Sequence
  GFP ncRNA
tctgttagtggagagggtga

SEQ ID NO: 8 - protein, 512 aa, Artificial Sequence
  JE1 amylase polypeptide
MKQQKRLYARLLTLLFALIFLLPHSAAAAHHNGTNGTMMQYFEWHLPNDGNHWNRLRDDASNLRNRGITAIWIPPAWKGTSQNDVGYGAYDLYDLGEFNQKGTVRTKYGTRSQLESAIHALKNNGVQVYGDVVMNHKGGADATENVLAVEVNPNNRNQEISGDYTIEAWTKFDFPGRGNTYSDFKWRWYHFDGVDWDQSRQFQNRIYKFRGKAWDWEVDSENGNYDYLMYADVDMDHPEVVNELRRWGEWYTNTLNLDGFRIDAVKHIKYSFTRDWLTHVRNATGKEMFAVAEFWKNDLGALENYLNKTNWNHSVFDVPLHYNLYNASNSGGNYDMAKLLNGTVVQKHPMHAVTFVDNHDSQPGESLESFVQEWFKPLAYALILTREQGYPSVFYGDYYGIPTHSVPAMKAKIDPILEARQNFAYGTQHDYFDHHNIIGWTREGNTTHPNSGLATIMSDGPGGEKWMYVGQNKAGQVWHDITGNKPGTVTINADGWANFSVNGGSVSIWVKR

SEQ ID NO: 9 - DNA, 3727 bp, Artificial Sequence
  plasmid pE194
gatcagtacaagaaagatactgtatttcataaacaggaactgcaagaagttaaggatgagttacagaaggcaaataagcagttacagagtggaatagagcatatgaggtctacgaaaccctttgattatgaaaatgagcgtacaggtttgttctctggacgtgaagagactggtagaaagatattaactgctgatgaatttgaacgcctgcaagaaacaatctcttctgcagaacggattgttgatgattacgaaaatattaagagcacagactattacacagaaaatcaagaattaaaaaaacgtagagagagtttgaaagaagtagtgaatacatggaaagaggggtatcacgaaaaaagtaaagaggttaataaattaaagcgagagaatgatagtttgaatgagcagttgaatgtatcagagaaatttcaagctagtacagtgactttatatcgtgctgcgagggcgaatttccctgggtttgagaaagggtttaataggcttaaagagaaattctttaatgattccaaatttgagcgtgtgggacagtttatggatgttgtacaggataatgtccagaaggtcgatagaaagcgtgagaaacagcgtacagacgatttagagatgtagaggtacttttatgccgagaaaactttttgcgtgtgacagtccttaaaatatacttagagcgtaagcgaaagtagtagcgacagctattaactttcggttgcaaagctctaggatttttaatggacgcagcgcatcacacgcaaaaaggaaattggaataaatgcgaaatttgagatgttaattaaagacctttttgaggtctttttttcttagatttttggggttatttaggggagaaaacataggggggtactacgacctcccccctaggtgtccattgtccattgtccaaacaaataaataaatattgggtttttaatgttaaaaggttgttttttatgttaaagtgaaaaaaacagatgttgggaggtacagtgatagttgtagatagaaaagaagagaaaaaagttgctgttactttaagacttacaacagaagaaaatgagatattaaatagaatcaaagaaaaatataatattagcaaatcagatgcaaccggtattctaataaaaaaatatgcaaaggaggaatacggtgcattttaaacaaaaaaagatagacagcactggcatgctgcctatctatgactaaattttgttaagtgtattagcaccgttattatatcatgagcgaaaatgtaataaaagaaactgaaaacaagaaaaattcaagaggacgtaattggacatttgttttatatccagaatcagcaaaagccgagtggttagagtatttaaaagagttacacattcaatttgtagtgtctccattacatgatagggatactgatacagaaggtaggatgaaaaaagagcattatcatattctagtgatgtatgagggtaataaatcttatgaacagataaaaataattacagaagaattgaatgcgactattccgcagattgcaggaagtgtgaaaggtcttgtgagatatatgcttcacatggacgatcctaataaatttaaatatcaaaaagaagatatgatagtttatggcggtgtagatgttgatgaattattaaagaaaacaacaacagatagatataaattaattaaagaaatgattgagtttattgatgaacaaggaatcgtagaatttaagagtttaatggattatgcaatgaagtttaaatttgatgattggttcccgcttttatgtgataactcggcgtatgttattcaagaatatataaaatcaaatcggtataaatctgaccgatagattttgaatttaggtgtcacaagacactcttttttcgcaccagcgaaaactggtttaagccgactgcgcaaaagacataatcgattcacaaaaaataggcacacgaaaaacaagttaagggatgcagtttatgcatcccttaacttacttattaaataatttatagctattgaaaagagataagaattgttcaaagctaatattgtttaaatcgtcaattcctgcatgttttaaggaattgttaaattgattttttgtaaatattttcttgtattctttgttaacccatttcataacgaaataattatacttttgtttatctttgtgtgatattcttgatttttttctacttaatctgataagtgagctattcactttaggtttaggatgaaaatattctcttggaaccatacttaatatagaaatatcaacttctgccattaaaagtaatgccaatgagcgttttgtatttaataatcttttagcaaacccgtattccacgattaaataaatctcattagctatactatcaaaaacaattttgcgtattatatccgtacttatgttataaggtatattaccatatattttataggattggtttttaggaaatttaaactgcaatatatccttgtttaaaacttggaaattatcgtgatcaacaagtttattttctgtagttttgcataatttatggtctatttcaatggcagttacgaaattacacctctttactaattcaagggtaaaatggccttttcctgagccgatttcaaagatattatcatgttcatttaatcttatatttgtcattattttatctatattatgttttgaagtaataaagttttgactgtgttttatatttttctcgttcattataaccctctttaatttggttatatgaattttgcttattaacgattcattataaccacttattttttgtttggttgataatgaactgtgctgattacaaaaatactaaaaatgcccatattttttcctccttataaaattagtataattatagcacgagctctgataaatatgaacatgatgagtgatcgttaaatttatactgcaatcggatgcgattattgaataaaagatatgagagatttatctaatttcttttttcttgtaaaaaaagaaagttcttaaaggttttatagttttggtcgtagagcacacggtttaacgacttaattacgaagtaaataagtctagtgtgttagactttatgaaatctatatacgtttatatatatttattatccggaggtgtagcatgtctcattcaattttgagggttgccagagttaaaggatcaagtaatacaaacgggatacaaagacataatcaaagagagaataaaaactataataataaagacataaatcatgaggaaacatataaaaattatgatttgattaacgcacaaaatataaagtataaagataaaattgatgaaacgattgatgagaattattcagggaaacgtaaaattcggtcagatgcaattcgacatgtggacggactggttacaagtgataaagatttctttgatgatttaagcggagaagaaatagaacgattttttaaagatagcttggagtttctagaaaatgaatacggtaaggaaaatatgctgtatgcgactgtccatctggatgaaagagtcccacatatgcactttggttttgtccctttaacagaggacgggagattgtctgcaaaagaacagttaggcaacaagaaagactttactcaattacaagatagatttaatgagtatgtgaatgagaaaggttatgaacttgaaagaggcacgtccaaagaggttacagaacgagaacataaagcgatg

SEQ ID NO: 10 - DNA, 1653 bp, Artificial Sequence
  JE1zyn expression cassette
ttgttaaaaattcggaatatttatacaatatcatatgtatcacattgaaagggaggagaatcatgaaacaacaaaaacggctttacgcccgattgctgacgctgttatttgcgctcatcttcttgctgcctcattctgcagccgcggcacatcacaatggtacgaatgggacgatgatgcagtatttcgagtggcatctgccgaatgacggcaaccattggaacaggctgcgggacgacgcgtccaatttaaggaatcgcggcataactgcgatctggataccaccggcgtggaagggtacgtcacaaaacgacgtgggatatggagcgtacgacctctacgatttaggagaattcaaccagaaaggcacagttcgaacaaagtacgggacgcgctctcaattggagtcagcgatccatgctctgaaaaacaacggcgttcaagtttatggcgatgttgtcatgaaccataaaggcggtgcagatgcgacagaaaatgtgctcgcagttgaagtaaatccaaacaatcgaaaccaagagatttccggagactatacgatcgaagcctggacaaaatttgattttccaggtcggggcaacacatatagtgattttaaatggcgctggtaccatttcgacggcgtagattgggatcagtcacggcaattccaaaaccgaatctataagtttagaggcaaagcttgggactgggaagttgattcggaaaacggaaactatgattatttaatgtatgcggacgtggatatggatcatccggaggtggtgaacgaacttcgccgttggggggaatggtataccaatacgttaaacctggatggatttcgcatagatgccgtgaaacatattaaatactcatttacccgggattggttaacgcatgtccggaacgccacgggtaaagagatgttcgccgtcgcggaattttggaaaaatgacctgggcgccttggaaaattaccttaacaaaacgaactggaatcacagcgtctttgacgtaccgcttcactacaacttatataatgcatctaactcaggaggcaattatgacatggccaagcttttgaatggaacggtggttcaaaagcacccgatgcatgcagtgacgttcgtcgataaccatgattcacagcctggggagtccctcgaaagtttcgtccaggagtggttcaaaccattagcatatgcccttattttgacgagggaacaaggatatcctagtgttttttacggcgactattatggaatcccgacacattctgtgccggccatgaaggcaaaaatcgatccaatcttggaagcgcgtcaaaacttcgcctatgggacgcaacatgattactttgaccaccataatattattggatggacacgcgaagggaataccacacaccccaattcaggattagcaacaattatgtcggacggtccagggggcgaaaaatggatgtatgtcggacaaaacaaagcaggccaagtgtggcatgacataacaggcaataaaccggggacagtgacgattaacgcagatggctgggcaaatttttcagtcaacggaggttcggtctccatttgggtgaaaagataaatcaataaaaaaacgctgtgcggttaaagggcacagcgttttttttgtgtat

SEQ ID NO: 11 - DNA, 1790 bp, Bacillus subtilis
cagccagagaacagcttgcaaagccaatataaaggtgacaacggggtaaagcctgataaatgtttgaaaattttcagtccgtataaacattccgagccccttttcttcattttggtatatctatagtaacacgtttatgagacaaaccatttaaatagtacgtacgcaaaggaggttatcatacatgatttacggcattgggctggacattaccgagcttaaacggatcgcctctatggctgggcgccagaaaaggtttgccgagcggattttgacgcgaagcgagcttgaccaatactatgagctttcagagaaaagaaaaaacgaatttctcgcgggcagattcgcggcaaaagaagcgttctcgaaagcatttggcaccggcattgggaggcagctcagctttcaggacattgaaattaggaaagaccaaaatggcaagccctatatcatttgtacgaaactgagccaggccgccgttcacgtatcgatcactcatacaaaagaatacgctgccgcgcaggttgtgattgaaaggttgtcaagctagtctgcatattagggaaaccccactcatatatttgatagtgcattaagggagacaagttgtttgaggcttttatggtacgcatctgttctgcctaaacgtgtaccgagcgtaccgttaaagtcaaacaagcgatttcttccttttacatcaattgagaaaaaggggttgaaaaaggtgagaaaaagctttgttttgcttttaacgggactgcttgctgttcttattctttctgcctgcgggcaaaaaacacagcaagatattgtggccgggttagatgaaaaggcgaaagaatacacctcgtacaaagcaaaagcgaaaatgaccatcgagacagggagtgagcctcaagtatacaatgtggaaatctggcacaaaaagccgtctctgtacagggtttatttagaaaatccgaaaaaggaccaaaaccaagtcattttacgaaatgaaaacggagtattcgttctcacgccgtctttaaataagagcttcagatttcaaagcgattggccgaacaacagcagccaggtttatctgtttgaatcgctcgtaaaggatgttcaaaacgattcggatgcagttttcacagcgaaagaaaagaaatacgtatttgaaacaaaaacaaactatcagcataacaaaatgctgcccacgcaagaaatcacatttaacaaaaaagatatgagcccgtcatctgtcaaagtgatggatactgaccggaaagtgatggtcaaagtagaattcagcagctttgaatttaataaacaatttgataaagaatcatttgatgaaaagaaaaatatgaccctttctcaaatggatgtcgccacaagcgcaaagccttccgatacatttgcggtcaaaacgccgctggaactgccgcttggcgtcaagctgcttgaagaaaaagatatatctactgaagacgggaagcgcatcatcatgacgtacggaggagaaaaatcatttacgttaattcaggaaaaagcccagattgccaaggcttcctcctccgttacgctgaacggcgaaccggtaaatctcggctacaccatcggcgccctgtcggatgcatcattatcatggacatatgacggcgtagattaccttctctcttctaaagatctttctaaagaggaaatggtgacagtagcgaaaagcatgcagggacaatcatcgaaataaccgccaaaggccaaacatgatttggcctttttttcgttagacatcgtttccctttagc

SEQ ID NO: 12 - DNA, 2292 bp, Bacillus subtilis
ttagacatcgtttccctttagcctttaattttagtatgatatgtaaatgatattgaataaaagctaggaagtgtcgtaatgagcacaaaacctttttacagagatacgtgggcggaaattgacttgtccgcgataaaggaaaatgtcagcaatatgaaaaaacatatcggtgaacatgtccacttgatggcagttgtgaaagcaaacgcctacgggcatggtgatgcagaaacagcaaaggctgctcttgacgcaggtgcttcatgcttggccgtggccattttggatgaagcgatttcactgcgcaaaaagggattgaaggcgcctatattggtgcttggcgcggttcccccggagtatgtggcaatcgctgctgagtatgacgtgaccttaacaggttattctgttgaatggcttcaggaggcagcccgccacacgaaaaaaggttctcttcattttcatctgaaggtcgatacggggatgaacagacttggtgtaaaaacagaggaagaagttcagaacgtgatggcaattcttgaccgcaaccctcgtttaaagtgcaaaggggtatttacccattttgcgacagcggatgaaaaagaaagaggctatttcttaatgcagtttgagcgctttaaagagctgattgctccgctgccgttaaagaatctaatggtccactgcgcgaacagcgccgctggactccggctgaaaaaaggcttttttaatgcagtcagattcggcatcggcatgtatggccttcgcccgtctgctgacatgtcggacgagataccgtttcagctgcgtccggcatttaccctgcattcgacactgtcacatgtcaaactgatcagaaaaggcgagagcgtcagctacggagccgagtacacagcggaaaaagacacatggatcgggacggtgcctgtaggctatgcggacggctggctccgaaaattgaaagggaccgacatccttgtgaagggaaaacgcctgaaaattgccggccgaatttgcatggaccaatttatggtggagctggatcaggaatatccgccgggcacaaaagtcacattaataggccggcagggggatgaatatatttccatggatgagattgcaggaaggctcgaaaccattaactatgaggtggcctgtacaataagttcccgtgttccccgtatgtttttggaaaatgggagtataatggaagtaagaaatcctttattgcaggtaaatataagcaattaacttacctaaatggagaattcataaaacagctttgcttgaagagtgaataatggtatcattatcactggatggataaaagagtcgtatccgtaagtttggtggaggtgtatgttttttgtctgaatccagcgcaagaaccgaaatgaaaatcagcttgcccgaaaacctagtagctgaattggatggtgtagcgatgcgggagaaacgaagcagaaacgaactgatatcacaagcagtgagagcgtatgtcagcgaacgaacaactcgtcacaaccgtgatttgatgagacgcggctatatggaaatggcgaaaatcaacctgaatatttcttctgaggctcactttgcggagtgcgaggctgaaacgacagttgagcgcttagtcagcggaggataatcatttgattgtgaaacgcggcgatgtttattttgctgatttatctcctgttgttggctcagagcaaggcggggtgcgcccggttttagtgatccaaaatgacatcggaaatcgcttcagcccaactgctattgttgcagccataacagcacaaatacagaaagcgaaattaccaacccacgtcgaaatcgatgcaaaacgctacggttttgaaagagattccgttattttgctggagcaaattcggacgattgacaagcaaaggttaacggataagattactcatctggatgatgaaatgatggataaggttgatgaagccttacaaatcagtttggcactcattgatttttagacatatttgcaggttgctcaaatagagcaactttttttgttttcaaaaaacataaacgatataatagtgaaataacgaaaaaatatgttgttttttattgggaggtaagcgaatttgatgtcgaaccagactgtataccagttcattgccgaaaatcaaaatgaactgctccaactgtggactgacacactaaaagaattaagcgagcaagaatcctatcagctgactgaccaagtgtatgaaaatatatctaaagaatatatcgacattctgctgctgtctgttaaggacg

SEQ ID NO: 13 - DNA, 2089 bp, Artificial Sequence
  Up_frag sequence for HTP integration into alr
gaaggaaacgcgcttttcccagcatgcgttccaaagccggggcaaataaaaagatggacatggaattaaacagcagatgtgtgaaaccggcgtgcagcaaaatcggtgtgataagccgccaccattcaccgtttgcgacacctagattatagcctgtgacggtatccctccataacacgactgaatgggcaggaagtgaaaaaaacagccagagaacagcttgcaaagccaatataaaggtgacaacggggtaaagcctgataaatgtttgaaaattttcagtccgtataaacattccgagccccttttcttcattttggtatatctatagtaacacgtttatgagacaaaccatttaaatagtacgtacgcaaaggaggttatcatacatgatttacggcattgggctggacattaccgagcttaaacggatcgcctctatggctgggcgccagaaaaggtttgccgagcggattttgacgcgaagcgagcttgaccaatactatgagctttcagagaaaagaaaaaacgaatttctcgcgggcagattcgcggcaaaagaagcgttctcgaaagcatttggcaccggcattgggaggcagctcagctttcaggacattgaaattaggaaagaccaaaatggcaagccctatatcatttgtacgaaactgagccaggccgccgttcacgtatcgatcactcatacaaaagaatacgctgccgcgcaggttgtgattgaaaggttgtcaagctagtctgcatattagggaaaccccactcatatatttgatagtgcattaagggagacaagttgtttgaggcttttatggtacgcatctgttctgcctaaacgtgtaccgagcgtaccgttaaagtcaaacaagcgatttcttccttttacatcaattgagaaaaaggggttgaaaaaggtgagaaaaagctttgttttgcttttaacgggactgcttgctgttcttattctttctgcctgcgggcaaaaaacacagcaagatattgtggccgggttagatgaaaaggcgaaagaatacacctcgtacaaagcaaaagcgaaaatgaccatcgagacagggagtgagcctcaagtatacaatgtggaaatctggcacaaaaagccgtctctgtacagggtttatttagaaaatccgaaaaaggaccaaaaccaagtcattttacgaaatgaaaacggagtattcgttctcacgccgtctttaaataagagcttcagatttcaaagcgattggccgaacaacagcagccaggtttatctgtttgaatcgctcgtaaaggatgttcaaaacgattcggatgcagttttcacagcgaaagaaaagaaatacgtatttgaaacaaaaacaaactatcagcataacaaaatgctgcccacgcaagaaatcacatttaacaaaaaagatatgagcccgtcatctgtcaaagtgatggatactgaccggaaagtgatggtcaaagtagaattcagcagctttgaatttaataaacaatttgataaagaatcatttgatgaaaagaaaaatatgaccctttctcaaatggatgtcgccacaagcgcaaagccttccgatacatttgcggtcaaaacgccgctggaactgccgcttggcgtcaagctgcttgaagaaaaagatatatctactgaagacgggaagcgcatcatcatgacgtacggaggagaaaaatcatttacgttaattcaggaaaaagcccagattgccaaggcttcctcctccgttacgctgaacggcgaaccggtaaatctcggctacaccatcggcgccctgtcggatgcatcattatcatggacatatgacggcgtagattaccttctctcttctaaagatctttctaaagaggaaatggtgacagtagcgaaaagcatgcagggacaatcatcgaaataaccgccaaaggccaaacatgatttggcctttttttcgttagacatcgtttccctttagctgctgtccagactgtccgctgtgtaaaaaaaaggaataaaggggggttgacattattttactgatatgtataatataatttgtataagaaaatg

SEQ ID NO: 14 - DNA, 2430 bp, Artificial Sequence
  Down_frag sequence for HTP integration into alr
tcaataaaaaaacgctgtgcggttaaagggcacagcgtttttttgtgtatgtcgattcacaaaaataggcacacgaaaaacttagacatcgtttccctttagcctttaattttagtatgatatgtaaatgatattgaataaaagctaggaagtgtcgtaatgagcacaaaacctttttacagagatacgtgggcggaaattgacttgtccgcgataaaggaaaatgtcagcaatatgaaaaaacatatcggtgaacatgtccacttgatggcagttgtgaaagcaaacgcctacgggcatggtgatgcagaaacagcaaaggctgctcttgacgcaggtgcttcatgcttggccgtggccattttggatgaagcgatttcactgcgcaaaaagggattgaaggcgcctatattggtgcttggcgcggttcccccggagtatgtggcaatcgctgctgagtatgacgtgaccttaacaggttattctgttgaatggcttcaggaggcagcccgccacacgaaaaaaggttctcttcattttcatctgaaggtcgatacggggatgaacagacttggtgtaaaaacagaggaagaagttcagaacgtgatggcaattcttgaccgcaaccctcgtttaaagtgcaaaggggtatttacccattttgcgacagcggatgaaaaagaaagaggctatttcttaatgcagtttgagcgctttaaagagctgattgctccgctgccgttaaagaatctaatggtccactgcgcgaacagcgccgctggactccggctgaaaaaaggcttttttaatgcagtcagattcggcatcggcatgtatggccttcgcccgtctgctgacatgtcggacgagataccgtttcagctgcgtccggcatttaccctgcattcgacactgtcacatgtcaaactgatcagaaaaggcgagagcgtcagctacggagccgagtacacagcggaaaaagacacatggatcgggacggtgcctgtaggctatgcggacggctggctccgaaaattgaaagggaccgacatccttgtgaagggaaaacgcctgaaaattgccggccgaatttgcatggaccaatttatggtggagctggatcaggaatatccgccgggcacaaaagtcacattaataggccggcagggggatgaatatatttccatggatgagattgcaggaaggctcgaaaccattaactatgaggtggcctgtacaataagttcccgtgttccccgtatgtttttggaaaatgggagtataatggaagtaagaaatcctttattgcaggtaaatataagcaattaacttacctaaatggagaattcataaaacagctttgcttgaagagtgaataatggtatcattatcactggatggataaaagagtcgtatccgtaagtttggtggaggtgtatgttttttgtctgaatccagcgcaagaaccgaaatgaaaatcagcttgcccgaaaacctagtagctgaattggatggtgtagcgatgcgggagaaacgaagcagaaacgaactgatatcacaagcagtgagagcgtatgtcagcgaacgaacaactcgtcacaaccgtgatttgatgagacgcggctatatggaaatggcgaaaatcaacctgaatatttcttctgaggctcactttgcggagtgcgaggctgaaacgacagttgagcgcttagtcagcggaggataatcatttgattgtgaaacgcggcgatgtttattttgctgatttatctcctgttgttggctcagagcaaggcggggtgcgcccggttttagtgatccaaaatgacatcggaaatcgcttcagcccaactgctattgttgcagccataacagcacaaatacagaaagcgaaattaccaacccacgtcgaaatcgatgcaaaacgctacggttttgaaagagattccgttattttgctggagcaaattcggacgattgacaagcaaaggttaacggataagattactcatctggatgatgaaatgatggataaggttgatgaagccttacaaatcagtttggcactcattgatttttagacatatttgcaggttgctcaaatagagcaactttttttgttttcaaaaaacataaacgatataatagtgaaataacgaaaaaatatgttgttttttattgggaggtaagcgaatttgatgtcgaaccagactgtataccagttcattgccgaaaatcaaaatgaactgctccaactgtggactgacacactaaaagaattaagcgagcaagaatcctatcagctgactgaccaagtgtatgaaaatatatctaaagaatatatcgacattctgctgctgtctgttaaggacgaaaatgctgctgaaagccaaatcagcgaactggcgcttcgagcggtccaaatcggac

SEQ ID NO: 15 - DNA, 795 bp, Bacillus subtilis
atgttacgttcactttattctggaatcagcggtatgaaaaactttcaaacaaagcttgacgtaatcggtaacaacattgccaacgtaaatacagtaggattcaaaaagagccgtgtgacctttaaagatatggtaagccaaacgattgccggcggttctgctgctggtgcaacaatcggcggaacaaactctaagcaaatcggtttaggttcatcatctggcacaattgatacaattcattcaacaagcgcgacacaaagcacaggaagaacacttgatttagcaattgacggcgacggttacttccgaattgatacaggtgacgggacagcttatacaagagcagggaacttctatttagataatactggaacactggttacaggtgatggctaccatgtactaaatatgaatggcggaacaattaaaattcctactgatgctcaaagctttagcattggttctgatggaaaggtgtcaattgttgatgctgaggggaaaacacaagacgggggacaaatcgggattgttacttttgccaacagtgatggattggataaaatcggaagcaacctataccgtgaatctttaaactctggaacggccagtgcagcgaaccagcctggtgacggcggaactggtgctcttaaatcaggatttctcgaaatgtctaacgtcgatttaactgacgaatttactgaaatgatcgtagcccagcgcggattccaatcaaactcaaaaattattacaacatctgatgaaatccttcaagaactggttaaccttaaacgttaa

SEQ ID NO: 16 - DNA, 780 bp, Bacillus subtilis
atgaattcaattattgacttatttcctgcttttttattggtttttattagaatctctgctttttttgtaacgattccgctatttggacatcgaaatgtgccagctgttcatcgcataggttttgctttttttcttgcggtcatttgtttcagcaccattgataaaccgccctctttggagatagatgagcactatatgcttttggcgtttaaagaagctttggtcggcctatgtctgggtttaattgcttatatgatgattgccgcagtgcagattgccggctcgtttattgattttcaaatgggattttcaatagcaaacgttattgatccgcaaaccggtgcacaaagtccgttgattggccagtttatctatacgatggcacttttgtttatgctgagtgtcaatgcccaccatttgttgctggatggtatttactacagctttcagtatatttcagttgatcaggcatttccgaatttcggcgatgaaaagtttgcctattttattgcgaaaagctttaatgcaatgtttattatcgcttttcaaatgtcagcgccggttgtggccagtttgtttctagttgatttagcattaggtatcgtggctcgaactgttccgcagttaaatgtatttgtggtcggtctgcctttaaagatcgctgtcagtttcattatgctcattgtatgtatgtccgttatcttcgtcgttgtccggaatgttttcagtttaacgattgaaacgatgcggaatcttttagcattggtcggtgtttcttaa

SEQ ID NO: 17 - DNA, 891 bp, Bacillus subtilis
atgaacagatatgaccaagcagcaactttacgggcgaaaatggaaaaacgtgagcgcgttctgccaatggtttattcacaaaaagcgaagacacttgctgtcatcagcggcaagggcggtgtcggaaaatccaatattaccttaaatatggcacttgcgctgcaggataaaggtaagaaggtgctgctcatcgaccttgatatcgggatggggaacattgatatattaataggaaattcatcgtctgccacgataatcgatgttttaaccgatcgtaagcctttgctgcagtcattatccgttggcccaaagggtttgcggtatatatcagggggaaccggtcttgatgtgatgtttcagctcgatcagagaaaatggacgttttttgccaatgaactttctcatgcattaagccagttcgattatgtgctgtttgacatgggagcaggcttatcaaaagatcaactgccttttattttatcagcagaagatattttgattattacaactcccgagccgacggccattatggacgcatacagcgctgtcaagcacttggttttgacagaaaataagctttcaatgaaggtggctgtcaatcggtgccgtgaccaaaaggaagggcttgacgcttttgcccgcctctcccgtacaattcatatgtttttggatgttcaggttcagtttgccggttccgtttctgacgatgtgatcgtgagcaaagcggttgtcgaacaggttccttttttcataaaaagccctcaggcaaaagccagccggtcagtccgtattttagcggacgccttgtttgaaagagaagaaacgagacacaaagaagacaaacagacatttattgagaaattatcttcttttttaatgaggagggcttaa

SEQ ID NO: 18 - DNA, 20 bp, Bacillus subtilis
acagtgatggattggataaa

SEQ ID NO: 19 - DNA, 20 bp, Bacillus subtilis
tagcattggttctgatggaa

SEQ ID NO: 20 - DNA, 20 bp, Bacillus subtilis
gctgttcatcgcataggttt

SEQ ID NO: 21 - DNA, 20 bp, Bacillus subtilis
ctatttggacatcgaaatgt

SEQ ID NO: 22 - DNA, 20 bp, Bacillus subtilis
tttattttatcagcagaaga

SEQ ID NO: 23 - DNA, 20 bp, Bacillus subtilis
gttcgattatgtgctgtttg

SEQ ID NO: 24 - protein, 129 aa, Bacillus subtilis
MSLFSGTIQNLENALSRADIKQKVITNNIANIDTPNYKAKKVSFQNLLDQESSRLEAIKTDYRHVDFSDTDSNYSIVASGDTSYQQNGNNVDVDKEMTELAQNQINYQALVERMNGKFNSLKTVLTGGK

SEQ ID NO: 25 - protein, 150 aa, Bacillus subtilis
MTAFHSLNVSASALTAQRVRMDVVSSNLANMDTTRAKQVNGEWVPYRRKMVSLQSKGESFSSILNSQMSGSGNAGNGVKVSKITEDDSDFNLVYDPTDPDANAEGYVQKPNVDPLKEMVDLVSSTRSYEANVTAMNATKGMLMKALEIGK

SEQ ID NO: 26 - protein, 140 aa, Bacillus subtilis
MTSISSEYKLPEKTNTVSTNNSSLGKDEFLKILMTQVQNQDPLNPIDDKEFISQMATFSSLEQMMNLNTTMTQFVENQDPFTTYVDWMGKEVSWTDGKSATDKTGTVSSVKHFKGNYYLVLDDGTEISPANVMSVGQSSK

SEQ ID NO: 27 - protein, 264 aa, Bacillus subtilis
MLRSLYSGISGMKNFQTKLDVIGNNIANVNTVGFKKSRVTFKDMVSQTIAGGSAAGATIGGTNSKQIGLGSSSGTIDTIHSTSATQSTGRTLDLAIDGDGYFRIDTGDGTAYTRAGNFYLDNTGTLVTGDGYHVLNMNGGTIKIPTDAQSFSIGSDGKVSIVDAEGKTQDGGQIGIVTFANSDGLDKIGSNLYRESLNSGTASAANQPGDGGTGALKSGFLEMSNVDLTDEFTEMIVAQRGFQSNSKIITTSDEILQELVNLKR

SEQ ID NO: 28 - protein, 507 aa, Bacillus subtilis
MTSTFMGLETARRALSAQQAALSTTANNVANANTDGYTRQRVSLEATDYFPAVSKNAEKTAGQMGTGVQGKSVERIRDIFLDYQYRLQNNSAGYYDTKAKALSQMEGVLNETDDSGLNSVLNSFWNSLQELSNNTNEESARSVVARKGQAVAETFNYISESLTNVQSNLKAELNTTVLDVNSLLSQLNSLNKQIAQVEPVGLLPNGLYDQRDLLIDKLSSMVDIKVSYNKSGGNALASAEGTVSIEILDKNKQSLGTVLDGKNYEVSELAANYDNETGLVSSISIGDTAVQAESFSSKGSLLGFIESYGYITADGQEKGVYPEMLSDLDNMALEFAKAFNEVHRNGVTKSGEQGGDFFDFTGGETEPAKGAAGKIKVADSIIDSKGANIAFSLTGAANDNANATKLANVLTGKITINGKETSVLDYYAGLIGELGIEAQEANRLASNTETQLNDADINRQQMSAVSLDEEMTNMIQFQHAYNAAARMVTLQDELLDKVINGMGVGGR

SEQ ID NO: 29 - protein, 298 aa, Bacillus subtilis
MRVTQGMIQQNSLRYIGSSYSKLDKLQSQISSGKKISKASDDPVVAMKSLKYNTQLSQVQQYKSNASQAFTWLENTETNITEGIDILSKVRELAVEAQNDTNGEPERQAIGVEVKQLKEQLLNIANTQVNGRYIFNGTNSDKPPVTDNGDGTYTISDTADVVVNISNNMSLKVNSDPKSAFGGTSESGQNVFEMLDSFEKALNSGSLDGMDSVLNDIDHFSDGMSAERSDLGARYNRLELVNTRLSAQEETATKVLSDNEDVELEEVITEFIAQQSVHRATLAVNAQIVQPTLIDFLK

SEQ ID NO: 30 - protein, 677 aa, Bacillus subtilis
MSTRDLSVLISVVLIVAMLVIPFPPWLLSILIIINISLALIVLLTTMNMQEALQFSIFPSLLLLLTLFRLGLNVSTTRSILSHGEGGKVVETFGNFVVGGNVLVGLVVFIILIIIQFIVITKGAERVSEVAARFTLDAMPGKQMSIDADLNAGMITEQEAKHRREKVAREADFYGAMDGASKFVKGDAIAGIIIVMINIIFGIVIGMLQQGMSIQEAASHFTMLTVGDGIVSQIPALLISTATGIVVTRAASEGNLGHDITGQLFAYPKLLYVAAATIMLLGIFTPIGILLTGPLAGLLAFGAYTLSKSGKEKEEVDEILEEEAEVDELKSPESVVQLLHIDPIEFEFGYGLIPLADANQGGDLLDRIVMIRRQLALELGLVIPVVRIRDNIALQPNEYRLKIKGNEVAKGELLLDHYLAMSPTPEDDLIEGIETVEPSFGLPAKWISEAVKDEADMLGYTVVDPASVVSTHITEKIKQHAHELIGRQETKQLIDHLKESYPVLVEEVTPNPLSVGDIQKVLAKLLKEKVSIRNLVTIFETLADYGKLTTDSDLLTEYTRQALAKQITAQFAKENEVLKVVTCSGRVEKAIADGVQQTEHGNYLSLEPDISESIVRSVAKEAEQLSLRQETAILLCSPPVRMYVKQLLERYFPDLPVLSYNELEANVEVQSIGVVDI

SEQ ID NO: 31 - protein, 360 aa, Bacillus subtilis
MKLRVDLQFFAGEKTEKATPKKRKDTRKKGQVAKSSDVNTAVSLLVIFLSLIAIGPYMRDRLLSFIETFYTESLTMKLSESNVHTLFVSLLKDMGMILAPILLVALVAGVVSNYMQVGFLFSAEVIQPKLEKLDPIKGFKRIYSMRAIVELIKSILKIVVVGFAAFAVLWLHYGEILRLPLLTPEEALSFVSKLTLWMGLSGAGALLILAGLDYLYQRFDYEKNIKMSKQDIKDEYKKSEGDPIIKSKIKQRQREMAMRRMMQEVPKADVIITNPTHYAIALKYDEEKMDAPYIVAKGVDHLALKIRKIAKEHDVMMVENRPLARALYDQVEIDQAVPEEFFKVLAEILAYVYKTKQKVY

SEQ ID NO: 32 - protein, 366 aa, Bacillus subtilis
MKIKKFTAASMQEAALLIRKELGNEAVILNSKKIKKRKWFGLVNKPAVEVIAVLDQDFLEKKTPQKAAEPKQTLKTPVSSPKIEERTYPPQIPAQQELGDFSAYQSVLPEPLRKAEKLLQETGIKESTKTNTLKKLLRFSVEAGGLTEENVVGKLQEILCDMLPSADKWQEPIHSKYIVLFGSTGAGKTTTLAKLAAISMLEKHKKIAFITTDTYRIAAVEQLKTYAELLQAPLEVCYTKEEFQQAKELFSEYDHVFVDTAGRNFKDPQYIDELKETIPFESSIQSFLVLSATAKYEDMKHIVKRFSSVPVNQYIFTKIDETTSLGSVFNILAESKIGVGFMTNGQNVPEDIQTVSPLGFVRMLCR

SEQ ID NO: 33 - protein, 298 aa, Bacillus subtilis
MQMNRYDQAATLRAKMEKRERVLPMVYSQKAKTLAVISGKGGVGKSNITLNMALALQDKGKKVLLIDLDIGMGNIDILIGNSSSATIIDVLTDRKPLLQSLSVGPKGLRYISGGTGLDVMFQLDQRKWTFFANELSHALSQFDYVLFDMGAGLSKDQLPFILSAEDILIITTPEPTAIMDAYSAVKHLVLTENKLSMKVAVNRCRDQKEGLDAFARLSRTIHMFLDVQVQFAGSVSDDVIVSKAVVEQVPFFIKSPQAKASRSVRILADALFEREETRHKEDKQTFIEKLSSFLMRRA

SEQ ID NO: 34 - protein, 270 aa, Bacillus subtilis
MLKGLYTATSAMIAQQRRTEMLSNNIANANTSGYKADQGSMRAFPEMLLSRIESKSPAGTSRTEIGSVNTGVYMQELKPLFTQGSLKSTDQPTDIALIENQVPMSAETNEKAALFYPVQTADGIRYSKSSTFSLNENNQLTINGRPILSTDRQPITVDNENFTVSENGTVTTNGRTAGQIDVRMAEDVRNLKRDGNDLYSTADGNDLPSAAGNNQVAYSLKQGVSELSNVDVTSAYTEMTEAYRSFEANQKVIQAYDKSMDKAANEIGKI

SEQ ID NO: 35 - protein, 269 aa, Bacillus subtilis
MLRSMLTASTTLNQLQQQIDTISSNLSNSNTTGYKAKDTNFSELVRQQFDQVDEKNEEVAKARKTPPGLRLGVGAMMSSRLVSDQGSIQKTDRDLDIAFTSPYQYLQVNVNGNRQYTRDGALYVTPSAANANQLQLVTGNGYPVLDENGNTVNIDSSMKNITINKNGTLTASDGNAVQRFNLGVVQVNNPQELKSEGNNLFSIDNAAAFEELNGANRQNIGMQQGSLEMSNVDISEQMTDLITSQRSYQLNSRTITMGDQMLGLINSVR

SEQ ID NO: 36 - protein, 498 aa, Bacillus subtilis
MVTRITGLASGMDIDDIVSKLMQTERAPLDKLTQKKQTLEWQRDSYREVNSKIKELQDYMSKNTLTYPSTYQSKTVTSSNESVLTATGSVSAPNSSSTVEVASLATAATYKANNYTGYTQGDYNLAFNVVAPGETTAKTVNISVTSADTIDNVISKLNSSDLGVSAFKDKIWNGTEYVETIAFSSKATGAGGSIQAADSATADFMSGQLGFSLDADNKLTAYKEGTNAKVTINGFEMEKLTNNFTVNGVTYSIKNTTAATGPVTTSVSTDVDGIYNQIKEFVDKYNELVDSLNEKLKEEKYRDYTPLTSEQKEAMSDKEVELWEEKAKSGLLRNDSSISTGTNQMRTDFYTQVNADGKTYQLTEFGITTSSAYQLRGHLEINEEKLKAKIAEDPQGVANLFTSGTNDSNYSDKGIMKRITNTLRSTVKSIEAKAGNSTMGASSYSIGKNLNSISTEITDMQDRLNTIENRYYSKFSAMDSAIQKMNEQASYLSQLLVQ

SEQ ID NO: 37 - protein, 106 aa, Bacillus subtilis
MINAISPFQVQNTQNTQNATNQVNNSQKTDSSNQTSFSELLKNSISSLNESQVASDNMTNALAAGKDVNLDEVMIAAQKASISLTAATEFRNKAVEAYQEIMRMQM

SEQ ID NO: 38 - protein, 536 aa, Bacillus subtilis
MNRTLMQMKNKTSEFWKNRSKLQKILMVSALAAIIIIGIIISVFASNSKMAPLYKDLSAEEAGQIKEELDAKKVPNELSNGGTVISVPEDQVDSLKVQMAAEGLPKTGSIDYSFFGQNAGFGLTDNEFDMVKVKATQTELSNLINEMDGIKNSKVMINLPKDAVFVGEEQSAASASIVLQIQPGYTLDQSQINGLYHLVSKSVPNLKEDNIVIMDQNSTYYDKSDSDAGSYADSYSSQQGIKSQVEKDIQKHVQSLLGTMMGQDKVVVSVTADIDFTKENRTEDIVEPVDKENMEGIAVSAEKVSETYQGDGAANGGTAGTGEEDVTNYKADGENTESGNYEKNSNKINYEVNRIHKEIAESPYKVRDLGIQVMVEPPDAKNTASLSTERQDDIQKILSTVVRTSLDKDETQNQNLSDADINNKIVVSVQPFDGKVNLDTNTEESSGIPLWAYIVGGVLIAAIIVLIIMLIRKKRAQEDEFEEYEYEVPQEPINLPDINEEENETAESVRRKQLEKMAKDKPEDFAKLLRSWLAED

SEQ ID NO: 39 - protein, 338 aa, Bacillus subtilis
MARRDQDKLTGKQKAAILMISLGLDVSASVYKHLTDEEIERLTLEISGVRSVDHQKKDEIIEEFHNIAIAQDYISQGGLSYARQVLEKALGEDKAENILNRLTSSLQVKPFDFARKAEPEQILNFIQQEHPQTMALILSYLDPVQAGQILSELNPEVQAEVARRIAVMDRTSPEIINEVERILEQKLSSAFTQDYTQTGGIEAVVEVLNGVDRGTEKTILDSLEIQDPDLAEEIKKRMFVFEDIVTLDNRAIQRVIRDVENDDLLLSLKVASEEVKEIVFNNMSQRMVETFKEEMEFMGPVRLKDVEEAQSRIVSIVRKLEEAGEIVIARGGGDDIIV

SEQ ID NO: 40 - protein, 253 aa, Bacillus subtilis
MILLSNIIKQESSISPQKEKRKLSIQEVRIDHSHLLQAEENPEALMARVKEEADRISEQANSHIENIRRQIEQEKNDWAAEKQKLIEEAKAEGFEQGVALGKAEAMKQYAELIGQANSITEMSRKAVEDKLEDANEEIVELAVALAKKVWQQKSDDKEAFLLLVQQVINEVKEYDDISIYVDPYYYETIFQQKDEIQQLLYKECRLGIYADEKAQKGTCYIETPFGRVDASVDTQLMQLKDKLLTALEAGAAE

SEQ ID NO: 41 - protein, 438 aa, Bacillus subtilis
MKTQSLIDCIEMTDSYKRYGKVKRVIGLMIESKGPASSIGDLCLIYAKGQSGKVIKAEVVGFQEENILLMPYLEAASIAPGSIVEATGESLRVKVGTGLIGQVIDAFGEPLDGKLLPKGLSPVSTEQSPPNPMKRPPIREKMGVGVRSIDSLLTVGKGQRIGIFAGSGVGKSTLMGMIAKQTEADLNVIALVGERGREVREFIEKDLGKEGLKRSIVVVATSDQPALMRLKAAYTATAIAEYFRDKGQNVMFMMDSVTRVAMAQREIGLAAGEPPTTKGYTPSVFAILPRLLERTGANEHGTITAFYTVLVDGDDMNEPIADTVRGILDGHIVLDRALANKGQFPAVNVLKSISRVMSNISTKQHLDAANKFRELLSTYQNSEDLINIGAYKRGSSREIDEAIQFYPQLIQFLKQGTDEPALLEESIAALTSLTGNEE

SEQ ID NO: 42 - protein, 147 aa, Bacillus subtilis
MAYQFRFQKLLELKENEKDQSLSEYQQSVSEFENVAEKLYENMSKKELLEQNKEKKLKSGMSVQEMRHYQQFVSNLDNTIYHYQKLVIMKRNQMNQKQEILTEKNIEVKKFEKMREKQFKMFALEDKAAEMKEMDDISIKQFMIQGH

SEQ ID NO: 43 - protein, 487 aa, Bacillus subtilis
MKLLELAGPLLQTTTGSAAKNMKSSQGVFQNWLMSEAGLKELSEQGKGTPNSEDQLLADALKKIGEWLNASPEDQDKQNADLLQTLSKLTGKQLDANALQMLQNLLQAVESKMSGGTDQLLTETEKIFSEAKTALSANDSASDINGALKSDKEQSNQENEVSEPAKELIYIQMFISQLVEGNKLTDLGNGNEAHAIYQNGDQFLSALEKKGVSQQLIQDLKQQIFTKAESSSKLYSMTASELKSFQSLMDQMSMLPQKGTKEWSLAESQLKAFLLSKSSESSQDFGKSVLTPLSQSSSSKNASDVSGSIQPVDSKSGLQMLFSGYRGIGGVQTLDLQQMSSDIPNAETKTVADQVINAWKQMKYTPFGRSTGSFTIRLNPEHLGFVTIKLTNENGMFQSKIIASSQSAKELLEQHLPQLKQSLPNMAVQIDRFTLPVQSGDQPIYGQLADEQKQQQEGQRQQRQKKQSNDFGDLLDEVSMVEMEEEE

SEQ ID NO: 44 - protein, 140 aa, Bacillus subtilis
MKKKLMIILLIILIVIGALGAAAYFVLGGKSEKSEAKKSIDEIVASSVDVEEITTNLKSDNIIRLAIKLETDSDKSKEELEKRDFQVKDAVISLLADTNADQIEGDKGKETFKKELKDKINSYLQEGKVEKVYITSFNLQ

SEQ ID NO: 45 - protein, 332 aa, Bacillus subtilis
MSGEVLSQNEIDALLSAISTGEMDAEELKKEEKEKKVKVYDFKRALRFSKDQIRSLTRIHDNFARLLTTHFSAQLRTYIHISVSSVDQVPYEEFIRSIPNMTILNLFDVHPMEGRIMMEVNPTIAYTMMDRVMGGIGISHNKVDSLTEIETKIISNLFENALGNYKEAWQSIADIEPEMTEFEVNPQFVQMVSPNETVVVISLNTQIGEISGVINLCIPHIVLEPLIPKLSVHYWMQSDRNEPKPEETKSLEKRIMTAQIPVVAELGTSELTIEEFLSLEVGDCITLDKSVTDPLTVLVGDKPKFLGQAGRVNRKQAVQILDHDIRGEQDGE

SEQ ID NO: 46 - protein, 221 aa, Bacillus subtilis
MNEFINIFSSSDPENVSSTVKLLLLLTVFSVAPGILILMTCFTRIVIVLSFVRTSLATQSMPPNQVLIGLALFLTFFIMAPTFSEINKEALTPLMDNKISLDEAYTKAEEPIKEFMSKHTRQKDLALFMNYAKMDKPESLKDIPLTTMVPAFAISELKTAFQIGFMIFIPFLIIDMVVASVLMSMGMMMLPPVMISLPFKILLFVLVDGWYLIVKSLLQSF

SEQ ID NO: 47 - protein, 89 aa, Bacillus subtilis
MSSEFVISMAEKAVYVTLMISGPLLAIALLVGLIVSIFQATTQIQEQTLAFIPKIVAVLLALIFFGPWMLSTILSFTTELFSNLNRFAG

SEQ ID NO: 48 - protein, 259 aa, Bacillus subtilis
MNSIIDLFPAFLLVFIRISAFFVTIPLFGHRNVPAVHRIGFAFFLAVICFSTIDKPPSLEIDEHYMLLAFKEALVGLCLGLIAYMMIAAVQIAGSFIDFQMGFSIANVIDPQTGAQSPLIGQFIYTMALLFMLSVNAHHLLLDGIYYSFQYISVDQAFPNFGDEKFAYFIAKSFNAMFIIAFQMSAPVVASLFLVDLALGIVARTVPQLNVFVVGLPLKIAVSFIMLIVCMSVIFVVVRNVFSLTIETMRNLLALVGVS

SEQ ID NO: 49 - protein, 378 aa, Bacillus subtilis
MENNRLSQDEIDALLNGTGSTLDEPEIPEVDDLSEMERDAIGEIGNISFGSSATALSTLLNQKVDITTPSVTVIPRSKISDAFPEPYVAIEVNYTEGFSGSNLLVVEQSDAAIIADLMIGGDGKGADPSLGEIHLSAVQEAMNQMMGSAATSMSTVFSKKIDISPPRVELLDVTEEKGTDRIPDDEMLVKVSFNLKVGELIDSSIMQLYPLTFAKDLISSLMNSESAEEEETVQPEVTYEQPKEPVTPEPRIEPKQQQQPPKRQGTAKKAAPVQVSPVEFSAFDPNEAVQAPIHNLDMLLDIPLSITVELGRTKRSVKEILELSAGSIIELDKLAGEPVDILVNQRIVAKGEVVVIEENFGVRVTDILSQAERINNLK

SEQ ID NO: 50 - protein, 219 aa, Bacillus subtilis
MKKSQYFIVFICFFVLFSVHPIAAAAADSDNSTVNEWFQKKDEKTADQSEQKKEKTTKTADETEGAAAPSVSAFDFVKMIFALLFVIVLIYGLVKLMNKRNRLLKPFQYVENIGGTSVGQNRSIQLIKVGKSVLVVGVGETIQLLKEIEDEKEIEVILSQHEEAMSSKIEWQKFVKPLKSSEHQPQQKLPSFSKALKEQLEELKQNRSEGKKKGPRHHE

SEQ ID NO: 51 - protein, 304 aa, Bacillus subtilis
MRINHNIAALNTLNRLSSNNSASQKNMEKLSSGLRINRAGDDAAGLAISEKMRGQIRGLEMASKNSQDGISLIQTAEGALTETHAILQRVRELVVQAGNTGTQDKATDLQSIQDEISALTDEIDGISNRTEFNGKKLLDGTYKVDTATPANQKNLVFQIGANATQQISVNIEDMGADALGIKEADGSIAALHSVNDLDVTKFADNAADTADIGFDAQLKVVDEAINQVSSQRAKLGAVQNRLEHTINNLSASGENLTAAESRIRDVDMAKEMSEFTKNNILSQASQAMLAQANQQPQNVLQLLR

SEQ ID NO: 52 - protein, 270 aa, Bacillus subtilis
MDKTSLIGIILAFVALSVGMVLKGVSFSALANPAAILIIIAGTISAVVIAFPTKEIKKVPTLFRVLFKENKQLTIEELIPMFSEWAQLARREGLLALEASIEDVDDAFLKNGLSMAVDGQSAEFIRDIMTEEVEAMEDRHQAGAAIFTQAGTYAPTLGVLGAVIGLIAALSHMDNTDELGHAISAAFVATLLGIFTGYVLWHPFANKLKRKSKQEVKLREVMIEGVLSVLEGQAPKVIEQKLLMYLPAKDRLKFAEQGEAQNGEKKEEEA

SEQ ID NO: 53 - protein, 261 aa, Bacillus subtilis
MARKKKKKHEDEHVDESWLVPYADILTLLLALFIVLYASSSIDAAKFQMLSKSFNEVFTGGTGVLDYSSVTPPENESDGIDEVKKEKEEKEKNKKEKEKAADQEELENVKSQVEKFIKDKKLEHQLETKMTSEGLLITIKDSIFFDSGKATIRKEDVPLAKEISNLLVINPPRNIIISGHTDNMPIKNSEFQSNWHLSVMRAVNFMGLLIENPKLDAKVFSAKGYGEYKPVASNKTAEGRSKNRRVEVLILPRGAAETNEK

SEQ ID NO: 54 - protein, 204 aa, Bacillus subtilis
MSGKKKESGKFRSVLLIIILPLMFLLIAGGIVLWAAGINVLKPIQDAAAKTPVLKELVPETENKKGAASSKDSSNTAALEKTIKDQKSEISILNKDLETSKSEIDRLNQKIRSLEKTAEDQKKSSEDHTEGSADSKASSENDKVISVYKSMDSGKAAKIIAQLKEQEALKILNGLSKKQLADILAKMTPEQAATYTEKIAASQE

SEQ ID NO: 55 - protein, 71 aa, Bacillus subtilis
MIKVTRLNGQPFTLNALFIEQIECFPDTTITLSNGKKFVVKEDEEAVLEKIAAFYRKIQIFAMDQGIEEPE

SEQ ID NO: 56 - protein, 120 aa, Bacillus subtilis
MAHRILIVDDAAFMRMMIKDILVKNGFEVVAEAENGAQAVEKYKEHSPDLVTMDITMPEMDGITALKEIKQIDAQARIIMCSAMGQQSMVIDAIQAGAKDFIVKPFQADRVLEAINKTLN

SEQ ID NO: 57 - protein, 357 aa, Bacillus subtilis
MIRVLVVDDSAFMRKMISDFLTEEKQIEVIGTARNGEEALKKIELLKPDVITLDVEMPVMNGTDTLRKIIEIYNLPVIMVSSQTEKGKECTINCLEIGAFDFITKPSGSISLDLYKIKEQLVERVVAAGLSGKRKRPVSQTVRPEPIVRAVVKPELSKPKPGTGRQIVCIGTSTGGPRALQKVIPKLPKDLNAPVVVVQHMPEGFTASLADRLNHLSDIQVKEAKDGEAALNGCVYIAPGGKNISVIKNSEGLQVVLDNHDTPSRHKPSADYLFRSVGKLTDYEKVAVIMTGMGSDGTAGLKDMLTAGNVKAIAESEESCVVYGMPKAAVKAGLIHEIKHVEDIAASITSCVKKERV

SEQ ID NO: 58 - protein, 672 aa, Bacillus subtilis
MDMNQYLDVFIDESKEHLQTCNEKLLLLEKDPTDLQLVHDIFRAAHTLKGMSATMGYTDLAHLTHLLENVLDAIRNGDMEVTSDWLDILFEALDHLETMVQSIIDGGDGKRDISEVSAKLDVNGAHAESAASAEPAEAQSSASDWEYDEFERTVIQEAEEQGFKRYEIKISLNENCMLKAVRVYMVFEKLNEVGEVAKTIPSAEVLETEDFGTDFQVCFLTHQSAEDIEQLINGVSEIEHVEVIQGAPLTSAEKPEESKQEDSPAAAVPAKQEKQKQPAKNDEQAKHSAGGSKTIRVNIDRLDSLMNLFEELVIDRGRLEQIAKELEHNELTETVERMTRISGDLQSIILNMRMVPVETVFNRFPRMIRQLQKELNKKIELSIIGAETELDRTVIDEIGDPLVHLIRNSIDHGIEAPETRLQKGKPESGKVVLKAYHSGNHVFIEVEDDGAGLNRKKILEKALERGVITEKEAETLEDNQIYELIFAPGFSTADQISDISGRGVGLDVVKNKLESLGGSVSVKSAEGQGSLFSIQLPLTLSIISVLLIKLEEETFAIPISSIIETAVIDRKDILQTHDREVIDFRGHIVPVVYLKEEFKIEDTRKDAEQLHIIVVKKGDKPTAFVVDSFIGQQEVVLKSLGDYLTNVFAISGATILGDGEVALIIDCNALII

SEQ ID NO: 59 - protein, 156 aa, Bacillus subtilis
MTAEIKTGEKMIVFMVNGKEYAISVTQVKSIEKWQKPTRVPGVEPYICGVINLRGVVTPVIDLRKRLNLPEYEITDETRIIIIAYRDIEVGWIVDEANDVITVHESEIESAPEGVQKDTDVSIEQIVKQENRLLNIIDANAVLDKESSQSAVPDQA

SEQ ID NO: 60 - protein, 209 aa, Bacillus subtilis
MSIFNGIKEEQMDILREVGNIGAGHSASAMAQLLNRKIDMEVPFAKLLSFDELVDFFGGADVPVASIFLRMEGDLTGSMFFIMPFFQAEQFIRELIGNPDFDIEDLGEDHMSSSALHELGNILAGSYLTALADLTKLQLYPSVPEVSLDMFGAVISEGLMELSQVGEHAIVVDTSIFDQSHQQELKAHMFMLPDYDSFEKLFVALGASL

SEQ ID NO: 61 - protein, 166 aa, Bacillus subtilis
MSTTEAVVIKVGIADVKIARFPDTIRTSGLGSCVGLVLYDKEKQTAGLVHVMLPDSTLSKTAELNRAKYADTAVQTTIDMLIEAGCRKFALKAKLAGGSEMFKFKSTNDLMKIGPRNVLAIKEQLSLFNIPIISEDTGGSSGRTIEFEPKSCMLHIRTVKQGEKTI

SEQ ID NO: 62 - protein, 254 aa, Bacillus subtilis
MQSLNYEDQVLWTRWKEWKDPKAGDDLMRRYMPLVTYHVGRISVGLPKSVHKDDLMSLGMLGLYDALEKFDPSRDLKFDTYASFRIRGAIIDGLRKEDWLPRTSREKTKKVEAAIEKLEQRYLRNVSPAEIAEELGMTVQDVVSTMNEGFFANLLSIDEKLHDQDDGENIQVMIRDDKNVPPEEKIMKDELIAQLAEKIHELSEKEQLVVSLFYKEELTLTEIGQVLNLSTSRISQIHSKALFKLKNLLEKVIQ

SEQ ID NO: 63 - protein, 167 aa, Bacillus subtilis
MSTLLWLLSFMLHGVLLYAVIILYTRLAAVKETEKQQKQILEETENTLAAFLLELKEENEKLIENKASSASQSDEESQKSGLQTSETYQERDPVQEAENLPEHIEGLITEVDRREELVNSEVQSFEDQVIELYEQGYSASQIAQKMKSGKTEIELFLKFRSKGVKDS

SEQ ID NO: 64 - DNA, 25 bp, Artificial Sequence
  oTK0558
gagtgccaatgattctgcttctgac

SEQ ID NO: 65 - DNA, 50 bp, Artificial Sequence
  oTK0578
atactgcaatcggatgcgattattgaatatattcccccagatgtttttat

SEQ ID NO: 66 - DNA, 47 bp, Artificial Sequence
  oTK0579
gacctgcaggcatgcaagcttagagtaatcgcttctccgctgtttta

SEQ ID NO: 67 - DNA, 24 bp, Artificial Sequence
  oTK0561
ctatcgtcagttcagatgtgccga

SEQ ID NO: 68 - DNA, 50 bp, Artificial Sequence
  oTK0577
ataaaaacatctgggggaatatattcaataatcgcatccgattgcagtat

SEQ ID NO: 69 - DNA, 47 bp, Artificial Sequence
  oTK0580
taaaacagcggagaagcgattactctaagcttgcatgcctgcaggtc

SEQ ID NO: 70 - DNA, 4055 bp, Bacillus subtilis
gagtgccaatgattctgcttctgacattaatggagcactgaagtctgataaagagcaatcaaatcaggaaaatgaagtatccgagcctgcaaaagagcttatctatatccagatgttcattagccagctcgtagaagggaataagctgactgatctcgggaatggcaatgaagctcatgccatttaccaaaatggagatcaattcctttctgcattagaaaagaagggtgtatctcaacaactaattcaggatcttaaacagcaaatattcactaaagctgagtcaagttcaaaactttactcgatgacagcgtcagagctgaaaagttttcaaagcttaatggatcaaatgtctatgcttcctcaaaaagggaccaaagaatggagtttagctgaaagccagctgaaagccttcttgttatcgaaatcatccgaatcatcacaagactttgggaagagtgttctcacaccgctctcgcagagttcatccagcaaaaatgcatcggatgtttctggaagtattcagcctgtagatagtaaatcaggactccaaatgctgttttcggggtatcggggcataggaggagttcaaacacttgatctgcagcagatgtcatctgatataccaaatgccgaaacaaaaacggtggctgatcaagtgattaatgcgtggaagcagatgaaatatacgcctttcggcagatcgacgggaagtttcactattcggctgaaccctgaacatttaggatttgtcacaatcaagctgacaaatgaaaatggaatgtttcagagtaaaatcatagcatctagccaatcagcaaaagaattgcttgaacagcatcttcctcagctgaagcaatcattgccgaatatggccgtccaaattgaccgttttactcttccggtccaaagcggggatcagccgatatacggccagcttgctgatgaacagaaacagcagcaagaggggcagagacagcaaagacagaaaaagcaatcaaatgattttggcgatctgctcgatgaagtgtcgatggttgaaatggaggaagaagaatgacttctataagttcagaatataaactgcctgaaaaaacgaacactgtgtcgacgaacaacagcagcttggggaaagacgagtttttaaaaatattaatgactcaagttcaaaaccaagatccgcttaacccgattgacgataaagaatttatcagccagatggcgactttttcaagcttggagcaaatgatgaatctgaatacgacaatgactcaattcgttgaaaaccaagatccgtttacaacgtatgttgattggatgggaaaagaagtatcttggactgatggtaaaagtgcaacagataaaacaggcacagtaagctctgttaaacattttaaaggaaattattatctcgttcttgatgatgggaccgagatcagtcctgcgaatgtcatgtctgtgggacaatcatctaaataaaaacatctgggggaatatattcaataatcgcatccgattgcagtataaatttaacgatcactcatcatgttcatatttatcagagctcgtgctataattatactaattttataaggaggaaaaaatatgggcatttttagtatttttgtaatcagcacagttcattatcaaccaaacaaaaaataagtggttataatgaatcgttaataagcaaaattcatataaccaaattaaagagggttataatgaacgagaaaaatataaaacacagtcaaaactttattacttcaaaacataatatagataaaataatgacaaatataagattaaatgaacatgataatatctttgaaatcggctcaggaaaaggccattttacccttgaattagtaaagaggtgtaatttcgtaactgccattgaaatagaccataaattatgcaaaactacagaaaataaacttgttgatcacgataatttccaagttttaaacaaggatatattgcagtttaaatttcctaaaaaccaatcctataaaatatatggtaatataccttataacataagtacggatataatacgcaaaattgtttttgatagtatagctaatgagatttatttaatcgtggaatacgggtttgctaaaagattattaaatacaaaacgctcattggcattacttttaatggcagaagttgatatttctatattaagtatggttccaagagaatattttcatcctaaacctaaagtgaatagctcacttatcagattaagtagaaaaaaatcaagaatatcacacaaagataaacaaaagtataattatttcgttatgaaatgggttaacaaagaatacaagaaaatatttacaaaaaatcaatttaacaattccttaaaacatgcaggaattgacgatttaaacaatattagctttgaacaattcttatctcttttcaatagctataaattatttaataagtaagttaagggatgcataaactgcatcccttaacttgtttttcgtgtgcctattttttgtgaatcgacctgcaggcatgcaagcttagagtaatcgcttctccgctgttttatgattaaagtaacccgtttgaacgggcagccctttacactgaatgcgctatttattgaacagattgaatgttttccggatactacaattactctgtcaaatggtaagaagtttgtagtaaaagaagatgaagaagctgttctggaaaagatcgcagctttctaccgaaaaatacaaatatttgcaatggatcaaggaatagaggaaccggaatgaagaaaaagttaatgatcatattactaattattcttatcgtaattggtgctctcggggcggcggcttattttgttttaggcggaaagtccgaaaaaagtgaagcgaaaaaaagtattgatgaaatcgttgcgtcttctgttgatgtagaagagatcacaacaaatttaaagtctgataacattatccgtcttgctattaaacttgaaactgattctgataaatcaaaagaagaacttgagaaacgtgatttccaagtgaaagacgctgttatatcactgctggctgatacgaatgctgatcagattgagggagacaagggaaaagaaacctttaagaaggaactaaaagataaaataaatagctacctccaagaaggaaaagtagaaaaagtgtatattacctcctttaatctgcaataaagcataatttgacagaatacggaggtgaggaaaatgtcaggagaagttctctcccaaaatgaaatagatgcactgctctctgcaatatcaactggtgaaatggacgctgaagagctgaaaaaagaagaaaaagagaagaaagtcaaggtttatgatttcaaacgtgcgctgcggttttctaaggatcagatccgcagtttaacgagaattcatgacaattttgcaagacttcttaccactcatttttctgctcagctcagaacctatattcacatatctgtcagttctgttgatcaggttccgtatgaggaatttatcagatcgattccaaacatgacgattctgaatctatttgatgttcatccgatggaaggaagaattatgatggaggtcaaccccacgatagcttatacgatgatggatcgagtcatgggcgggattggaatcagtcataacaaggttgacagtttgacagaaattgaaacaaaaatcatttctaatttatttgaaaatgcactgggtaattataaagaagcttggcagtcaattgctgatattgaaccggaaatgactgagtttgaagtgaatccgcaatttgttcagatggtatctcctaatgaaacagtcgtggtgatctcgctcaatactcaaattggtgaaatcagcggtgtcattaatctctgtatcccgcatattgtactcgagccgctcataccgaagctttcagtccactattggatgcaatcagaccgaaatgagccaaagcctgaggaaacaaagtcgcttgaaaaacgtatcatgacagcacaaatacctgtcgtggccgagctcggcacatctgaactgacgatag

SEQ ID NO: 71 - protein, 217 aa, Bacillus subtilis
MIEIGENVLLEYIEENELKKAKSKAVSIENNELLIAYPVDVVTGRTVILHNDMEVTVEFVGKDEVPYRFISRIKGKVKDKLQMICLEMPPREKMKRIQRRQYVRTDAVLDVQIQPGNEEEIRTLSYNISAGGIAVVLADGLSFQSGESLRLIIRLPEEEHTRQIETEAVVRRIFNDPKSEKRKMTLEYSEIAAGDQQALLQYCIRRQLNKRRKARME

SEQ ID NO: 72 - protein, 382 aa, Bacillus clausii
MMRKKSFWLGMLTAFMLVFTMAFSDSASAAEEAKEKYLIGFNEQEAVSEFVEQVEANDEVAILSEEEEVEIELLHEFETIPVLSVELSPEDVDALELDPAISYIEEDAEVTTMAQSVPWGISRVQAPAAHNRGLTGSGVKVAVLDTGISTHPDLNIRGGASFVPGEPSTQDGNGHGTHVAGTIAALNNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 73 - protein, 381 aa, Artificial Sequence
  artificial sequence, Nattokinase
MRSKKLWISLLFALTLIFTMAFSNMSAQAAGKSSTEKKYIVGFKQTMSAMSSAKKKDVISEKGGKVQKQFKYVNAAAATLDEKAVKELKKDPSVAYVEEDHIAHEYAQSVPYGISQIKAPALHSQGYTGSNVKVAVIDSGIDSSHPDLNVRGGASFVPSETNPYQDGSSHGTHVTGTIAALNNSIGVLGVAPSASLYAVKVLDSTGSGQYSWIINGIEWAISNNMDVINMSLGGPTGSTALKTVVDKAVSSGIVVAAAAGNEGSSGSTSTVGYPAKYPSTIAVGAVNSSNQRASFSSVGSELDVMAPGVSIQSTLPGGTYGAYNGTSMATPHVAGAAALILSKHPTWTNAQVRDRLESTATYLGNSFYYGKGLINVQAAAQ

SEQ ID NO: 74 - protein, 210 aa, Artificial Sequence
  artificial sequence, xylanase
MFKFVTKVLTVVIAATISFCLSAVPASANTYWQYWTDGGGTVNATNGPGGNYSVTWRDTGNFVVGKGWEIGSPNRTIHYNAGVWEPSGNGYLTLYGWTRNQLIEYYVVDNWGTYRPTGTHRGTVVSDGGTYDIYTTMRYNAPSIDGTQTFQQFWSVRQSKRPTGNNVSITFSNHVNAWRNAGMNLGSSWSYQVLATEGYQSSGRSNVTVW

SEQ ID NO: 75 - protein, 1065 aa, Artificial Sequence
  artificial sequence, xanthan lyase
MKKPLGKIVASTALLISVAFSSSIASAADEFDTLREKYKAMLNGGTTYNLSDPDIAARVNAITVTAQGYWDSMLKDPNRNRLWNDAPFGSDSTSITTTYRHLYDMALAYTTYGSSLQGNAALKADIISGLDWMNANQFYNGCSQYQNWWHWQIGGPMALNDIVALMYTELTATQISNYMAAIYYTQASVTMTGANRLWESQVIAISGILNKDSARVAAGRDGISALLPYVAKGDGFYNDGSFVQHTYYAYNGGYGSSLLSGIADLIFILNGSSWQVTDPNKNNVYRWIYDSYEPFIYKGNLMDMVRGREISRHGLQDDKAAVTVMASIIRLSQTAASADATAFKRMVKYWLLLDTDKTFLKAVSIDLIIAANQLVNDSTVTSRGELVKYKQFSGMDRAVQLRPGFGFGLSMFSSRIGNYESINAENNKGWHTGDGMTYLYNTDLSQFNDHFWATVDNYRLPGTTVLQKTTQTANSRSDKSWAGGTDILGQYGVSGMELHTVGKSLTAKKSWFMFDDEIVALGSGIASTDGIATETIVENRKLNSSGNNALIVNGTAKPGSLGWSETMTGTNYIHLAGSVPGSDIGYYFPGGAAVKGLREARSGSWSSLNKSASWKDSTLHTRNFMTLWFDHGMNPTNGSYSYVLLPNKTSSEVASYAATPQIEILENSSSAQAVKETQLNVTGINFWNDEPTTVGLVTSDRKASVMTKETASDFEISVSDPTQSNVGTIYIDVNKSATGLISKDNEITVIQYYPTMKFKVNVNNSLGKSYKVKFSLTGTPEENPDPIPIPNPYEAEDLPINAATDTPVVYNDANASGGKKLGFNNNAVGDYVEFSLDVTQPGTYDVKSRIMKSTNSGIYQLSINGTNVGSAQDMFWTTSELSKEFTMGSYSFSTPGSYLFRLTTTGKNVSSSGYKLMLDYFSLVSTGIDTTVIVDNADAAGVTKVGTWTGTNTQTDRYGADYIHDGNTGKGTKSVTFTPNVPISGTYQVYMMWAAHTNRATNVPVDVTHSGGTATLNVNQQGNGGVWNLLGTYSFNAGSTGAIKIRTDATNGYVVADAVKLVKVP

SEQ ID NO: 76 - DNA, 2327 bp, Artificial Sequence
  artifical sequence, Pcons-aprH-cat
ttgacattattttactgatatgtataatataatttgtataagaaaatgagagggagaggaaacatgaagaaaccgttggggaaaattgtcgcaagcaccgcactactcatttctgttgcttttagttcatcgatcgcatcggctgctgaagaagcaaaagaaaaatatttaattggctttaatgagcaggaagctgtcagtgagtttgtagaacaagtagaggcaaatgacgaggtcgccattctctctgaggaagaggaagtcgaaattgaattgcttcatgaatttgaaacgattcctgttttatccgttgagttaagcccagaagatgtggacgcgcttgaactcgatccagcgatttcttatattgaagaggatgcagaagtaacgacaatggcgcaatcagtgccatggggaattagccgtgtgcaagccccagctgcccataaccgtggattgacaggttctggtgtaaaagttgctgtcctcgatacaggtatttccactcatccagacttaaatattcgtggtggcgctagctttgtaccaggggaaccatccactcaagatgggaatgggcatggcacgcatgtggccgggacgattgctgctttaaacaattcgattggcgttcttggcgtagcgccgagcgcggaactatacgctgttaaagtattaggggcgagcggttcaggttcggtcagctcgattgcccaaggattggaatgggcagggaacaatggcatgcacgttgctaatttgagtttaggaagcccttcgccaagtgccacacttgagcaagctgttaatagcgcgacttctagaggcgttcttgttgtagcggcatctgggaattcaggtgcaggctcaatcagctatccggcccgttatgcgaacgcaatggcagtcggagctactgaccaaaacaacaaccgcgccagcttttcacagtatggcgcagggcttgacattgtcgcaccaggtgtaaacgtgcagagcacatacccaggttcaacgtatgccagcttaaacggtacatcgatggctactcctcatgttgcaggtgcagcagcccttgttaaacaaaagaacccatcttggtccaatgtacaaatccgcaatcatctaaagaatacggcaacgagcttaggaagcacgaacttgtatggaagcggacttgtcaatgcagaagcggcaacacgctaaggtaataaaaaaacacctccaagctgagtgcgggtatcagcttggaggtgcgtttattttttcagccgtatgacaaggtcggcatcaggtgtgacaacgcgtgatctagaccagttccctgagcttccgtcagtcggatcccattgcggattttcctcctctaatatgctcaacttaaatgacctattcaataaatctattatgctgctaaatagtttataggacaaataagtatactctaatgacctataaaagatagaaaattaaaaaatcaagtgttcgcttctctctcacggagctgtaatataaaaaccttcttcagctaacggggcaggttagtgacattagaaaaccgactgtagaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatttcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatcctttttt

SEQ ID NO: 77 - DNA, 2338 bp, Artificial Sequence
  artifical sequence, Pcons-nattokinase-cat
ttgacattattttactgatatgtataatataatttgtataagaaaatgagagggagaggaaacatgagaagcaaaaaattgtggatcagcttgttgtttgcgttaacgttaatctttacgatggcgttcagcaacatgtctgcgcaggctgccggaaaaagcagtacagaaaagaaatacattgtcggatttaagcagacaatgagtgccatgagttccgccaagaaaaaggatgttatttctgaaaaaggcggaaaggttcaaaagcaatttaagtatgttaacgcggccgcagcaacattggatgaaaaagctgtaaaagaattgaaaaaagatccgagcgttgcatatgtggaagaagatcatattgcacatgaatatgcgcaatctgttccttatggcatttctcaaattaaagcgccggctcttcactctcaaggctacacaggctctaacgtaaaagtagctgttatcgacagcggaattgactcttctcatcctgacttaaacgtcagaggcggagcaagcttcgttccttctgaaacaaacccataccaggacggcagttctcacggtacgcatgtcaccggtacgattgccgctcttaataactcaatcggtgttctgggcgtagcgccaagcgcatcattatatgcagtaaaagtgcttgattcaacaggaagcggccaatatagctggattattaacggcattgagtgggccatttccaacaatatggatgttatcaacatgagccttggcggacctactggttctacagcgctgaaaacagtagttgataaagcggtttccagcggtatcgtcgttgctgccgcagccggaaacgaaggttcatccggaagcacaagcacagtcggctaccctgcaaaatatccttctactattgcagtaggtgcggtaaacagcagcaaccaaagagcttcattctccagcgtaggttctgagcttgatgtaatggctcctggcgtgtccatccaaagcacacttcctggaggcacttacggcgcttataacggaacgtccatggcgactcctcacgttgccggagcagcagcgctaattctttctaagcacccgacttggacaaacgcgcaagtccgtgatcgtttagaaagcactgcaacatatcttggaaactctttctactatggaaaagggttaatcaacgtacaagcagctgcacaataataaggtaataaaaaaacacctccaagctgagtgcgggtatcagcttggaggtgcgtttattttttcagccgtatgacaaggtcggcatcaggtgtgacaacgcgtgatccagaccagttccctgagcttccgtcagtcggatcccattgcggattttcctcctctaatatgctcaacttaaatgacctattcaataaatctattatgctgctaaatagtttataggacaaataagtatactctaatgacctataaaagatagaaaattaaaaaatcaagtgttcgcttctctctcacggagctgtaatataaaaaccttcttcagctaacggggcaggttagtgacattagaaaaccgactgtagaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatttcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatccttttttaaaag

SEQ ID NO: 78 - DNA, 1817 bp, Artificial Sequence
  artificial sequence, Pcons-xylanase-cat
ttgacattattttactgatatgtataatataatttgtataagaaaatgagagggagaggaaacatgtttaagttcgttacgaaagttttgacggtagtaattgcagctacaattagtttttgtttgagtgcagtaccggcaagtgctaatacctattggcaatattggaccgatggtggtggaacagtaaatgctacaaatggacctggtggaaattacagtgtgacatggagagatacagggaactttgttgtcggtaaaggttgggaaatcggttcaccaaatcgaacgatccattacaatgctggtgtttgggaaccgtctggaaatggatatttgactctctatgggtggacaaggaatcagctcatagaatattatgtcgttgataattggggaacttacagacctactggaacccatcgaggcaccgttgtcagtgatgggggaacatatgatatctatacgactatgcgatacaatgcaccttccattgatggcacacaaacgttccaacagttctggagtgtgaggcaatcgaagagaccgactggaaataacgttagcattacgtttagcaaccacgtgaatgcgtggagaaatgcaggaatgaatctgggaagtagttggtcttaccaggtattagcaacagaaggctatcaaagtagcgggagatcgaatgtgacggtttggtagggtaataaaaaaacacctccaagctgagtgcgggtatcagcttggaggtgcgtttattttttcagccgtatgacaaggtcggcatcaggtgtgacaacgcgtgatctagaccagttccctgagcttccgtcagtcggatcccattgcggattttcctcctctaatatgctcaacttaaatgacctattcaataaatctattatgctgctaaatagtttataggacaaataagtatactctaatgacctataaaagatagaaaattaaaaaatcaagtgttcgcttctctctcacggagctgtaatataaaaaccttcttcagctaacggggcaggttagtgacattagaaaaccgactgtagaaagtacagtcggcattatctcatattataaaagccagtcattaggcctatctgacaattcctgaatagagttcataaacaatcctgcatgataaccatcacaaacagaatgatgtacctgtaaagatagcggtaaatatattgaattacctttattaatgaattttcctgctgtaataatgggtagaaggtaattactattattattgatatttaagttaaacccagtaaatgaagtccatggaataatagaaagagaaaaagcattttcaggtataggtgttttgggaaacaatttccccgaaccattatatttctctacatcagaaaggtataaatcataaaactctttgaagtcattctttacaggagtccaaataccagagaatgttttagatacaccatcaaaaattgtataaagtggctctaacttatcccaataacctaactctccgtcgctattgtaaccagttctaaaagctgtatttgagtttatcacccttgtcactaagaaaataaatgcagggtaaaatttatatccttcttgttttatgtttcggtataaaacactaatttcaatttctgtggttatactaaaagtcgtttgttggttcaaataatgattaaatatctcttttctcttccaattgtctaaatcaattttattaaagttcatttgatatgcctcctaaatttttatctaaagtgaatttaggaggcttacttgtctgctttcttcattagaatcaatcctttttt

SEQ ID NO: 79 - DNA, 5175 bp, Artificial Sequence
  artificial sequence, P4199-Xanthan lyase-cat
gcaacgttcgcagatgctgctgaagagattattaaaaagctgaaagcaaaaggctatcaattggtaactgtatctcagcttgaagaagtgaagaagcagagaggctattgaataaatgagtagaaagcgccatatcggcgcttttcttttggaagaaaatatagggaaaatggtacttgttaaaaattcggaatatttatacaatatcatatgtatcacattgaaaggaggggcctgctgtccagactgtccgctgtgtaaaaaaaaggaataaaggggggttgacattattttactgatatgtataatataatttgtataagaaaatggaggggccctcgaaacgtaagatgaaaccttagataaaagtgctttttttgttgcaattgaagaattattaatgttaagcttaattaaagataatatctttgaattgtaacgcccctcaaaagtaagaactacaaaaaaagaatacgttatatagaaatatgtttgaaccttcttcagattacaaatatattcggacggactctacctcaaatgcttatctaactatagaatgacatacaagcacaaccttgaaaatttgaaaatataactaccaatgaacttgttcatgtgaattatcgctgtatttaattttctcaattcaatatataatatgccaatacattgttacaagtagaaattaagacacccttgatagccttactatacctaacatgatgtagtattaaatgaatatgtaaatatatttatgataagaagcgacttatttataatcattacatatttttctattggaatgattaagattccaatagaatagtgtataaattatttatcttgaaaggagggatgcctaaaaacgaagaacattaaaaacatatatttgcaccgtctaatggatttatgaaaatcatttttatcagtttgaaaattatgtattatggagctctataaaaatgaggagggaaccgaatgaagaaaccgttggggaaaattgtcgcaagcaccgcactactcatttctgttgcttttagttcatcgatagcatcggctgcggacgagtttgacacgctaagggaaaagtataaggccatgctgaacggagggacaacctataatctctccgacccggatatagcggcgcgtgttaatgccattacggtgactgcccagggatactgggactccatgcttaaagatccgaaccgtaaccgtctttggaacgatgcaccctttggctcggattcgacttccatcaccacgacctacagacacctttatgatatggcgctagcttatacgacttatggctccagtctgcagggcaatgccgcacttaaagcggatattatcagcggtttggactggatgaatgccaatcaattttataatggctgcagccaatatcaaaactggtggcactggcaaattggcggtcccatggccttgaatgatatcgtggcattaatgtacacggagctaaccgcaacacaaatttccaattacatggcggccatttattacacccaagcgagtgttacgatgacgggggcaaaccggctatgggaaagtcaggttattgccatctccggaatcttgaataaggattccgccagagttgccgctggtcgggatggcatcagcgctttgctgccgtatgtcgccaagggtgacggattttacaacgatggatcattcgttcagcatacttattatgcttacaacggtggttatggttcaagcctgttatctggcattgcagacttgatatttattttgaatggctcttcatggcaggtaacggatcctaataaaaacaatgtataccgttggatttatgattcctacgagcctttcatctataaagggaatctgatggacatggtccgcggtagagagatctcaaggcatggattgcaggacgataaggcagccgtgactgtgatggcatcgatcattcgtctgtcacaaaccgctgcttccgccgatgctaccgcatttaagagaatggtgaaatattggctgctgctggatacggataagactttccttaaagcagtatcgattgatctgattattgccgcgaaccaactggtgaacgattccaccgttacctctcgaggggagctagtgaaatataaacaattctccggaatggaccgcgctgtacagcttagacctggcttcggttttgggcttagcatgttttccagccggatcggtaattatgagtcgattaatgcagagaacaacaaaggctggcataccggcgacggcatgacctacctttacaatactgacctgagtcagttcaatgaccatttctgggcaactgtggataattaccgattgccgggtaccacagtgctccagaagacgacgcaaaccgcgaacagccgcagcgacaaaagctgggccggaggaacggatattcttgggcaatatggtgtttccggcatggaactgcataccgtaggtaagagcctgacagccaagaaatcctggttcatgtttgacgatgagatcgtcgcgctgggttcaggtattgccagcaccgatggcatcgcaaccgaaacgattgtagagaatcgaaagctcaatagcagcggcaataatgcattgattgttaacgggacggcgaagccgggctcccttggatggtcggaaacaatgaccggaaccaattatattcatctagccggcagcgtacccggctccgatatcggttattattttcctggtggagcagcagtcaaaggcttgcgtgaagcccggtcgggaagctggagctcgctgaataagtccgcatcctggaaggactcgacattgcatacacgcaactttatgacgctttggttcgatcatggcatgaacccgacaaacggtagttattcttatgtgctgcttccgaataagaccagcagtgaggtggccagctatgctgcaacgcctcagatcgagattctggagaattctagctcggcgcaagcggtgaaggagacgcaattgaatgtcaccggaattaacttttggaacgatgagccaaccacggtgggcctggttacttccgatcggaaagcatccgttatgacaaaagaaacggctagtgatttcgagatatccgtttccgacccgacccaaagtaatgtggggaccatctatattgatgtcaacaaaagtgcaaccggattgatttcgaaggataatgaaataacggtcattcagtactacccaaccatgaagtttaaagtcaatgtaaacaattctctggggaagtcctataaagtaaagtttagcctgacaggaacacccgaggagaacccggatccaatcccgataccgaatccttacgaagcggaagatttgccaattaacgctgccacagatactcccgtggtttacaatgatgccaatgccagtggtggcaagaagcttggcttcaataacaatgcagtgggcgattatgtggagttcagtctggacgtcacacagcccggcacctacgatgtcaaatcccggattatgaaatcaacgaacagcgggatttatcagctgtctattaatgggaccaacgtagggagcgcgcaggatatgttctggacgacctccgagctgtctaaggagtttactatgggctcatacagcttcagcacacccgggagctatttgttccgattaacgacaaccggcaagaatgtcagttcttcaggatataagctgatgctggactattttagtctggtatcaacaggtattgatacaacggtgattgtggacaatgccgatgcagctggtgttacgaaggtgggtacttggaccggaaccaatacgcagaccgatcggtacggcgccgactacattcacgatgggaacacggggaaaggtacgaagagcgttacctttactccaaatgtacctatcagtggaacttatcaggtttacatgatgtgggctgcccatacgaatagggcaacgaatgttcccgtagacgtaacgcattcaggcggtacagcaacgctaaatgttaaccaacaaggtaatggtggtgtgtggaatttactgggtacgtatagctttaatgctgggtccacgggggctatcaagatccgtacggacgcgacgaatggatatgttgtagccgatgccgtgaagctggtaaaggtcccataaacgcgttaatcaataaaaaaacgctgtgcggttaaagggcacagcgtttttttgtgtatcggcaatagttacccttattatcaagataagaaagaaaaggatttttcgctacgctcaaatcctttaaaaaaacacaaaagaccacattttttaatgtggtctttattcttcaactaaagcacccattagttcaacaaacgaaaattggataaagtgggatatttttaaaatatatatttatgttacagtaatattgacttttaaaaaaggattgattctaatgaagaaagcagacaagtaagcctcctaaattcactttagataaaaatttaggaggcatatcaaatgaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccatggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataa

SEQ ID NO: 80 - DNA, 22 bp, Artificial Sequence
  citA - fwd oligo
gccaaacaacatggacgatatg

SEQ ID NO: 81 - DNA, 22 bp, Artificial Sequence
  citA - rev oligo
gcaatgatagaaggcgtgattg

SEQ ID NO: 82 - DNA, 20 bp, Artificial Sequence
  flgE - fwd oligo
tcggcggaacaaactctaag

SEQ ID NO: 83 - DNA, 21 bp, Artificial Sequence
  flgE - rev oligo
gtcgccgtcaattgctaaatc

SEQ ID NO: 84 - DNA, 20 bp, Artificial Sequence
  flgB - fwd oligo
ccttgagcagagcggatatt

SEQ ID NO: 85 - DNA, 20 bp, Artificial Sequence
  flgB - rev oligo
gacgcgaggattcttgatct

SEQ ID NO: 86 - DNA, 21 bp, Artificial Sequence
  fliR - fwd oligo
cggcctatgtctgggtttaat

SEQ ID NO: 87 - DNA, 20 bp, Artificial Sequence
  fliR - rev oligo
ccggtttgcggatcaataac

SEQ ID NO: 88 - DNA, 22 bp, Artificial Sequence
  artificial sequence, flhG - fwd oligo
gcgttctgccaatggtttattc

SEQ ID NO: 89 - DNA, 22 bp, Artificial Sequence
  artificial sequence, flhG - rev oligo
cagcgcaagtgccatatttaag

SEQ ID NO: 90 - DNA, 22 bp, Artificial Sequence
  artificial sequence, cheD - fwd oligo
gtcttgttcatgtcatgcttcc

SEQ ID NO: 91 - DNA, 21 bp, Artificial Sequence
  artificial sequence, cheD - rev oligo
agcatatcaatcgtggtctgt

(19) United States
(12) Patent Application Publication
(10) Pub. No.: US 2024/0052000 A1
(43) Pub. Date: Feb. 15, 2024

(54) MUTATED HOST CELLS WITH REDUCED CELL MOTILITY ON

(71) Applicants: Novozymes A/S, Bagsvaerd (DK); University of Copenhagen, Copenhagen (DK)

(72) Inventors: Thomas Krogh Kallehauge, Herlev (DK); Annaleigh Ohrt Fehler, Copenhagen (DK); Adrian Sven Geissler, Copenhagen (DK); Jan Gorodkin, Copenhagen (DK); Jeppe Vinther, Copenhagen (DK)

(73) Assignees: Novozymes A/S, Bagsvaerd (DK); University of Copenhagen, Copenhagen (DK)

(21) Appl. No.: 18/257,840

(22) PCT Filed: Dec. 15, 2021
(86) PCT No.: PCT/EP2021/085880

§ 371 Date: Jun. 15, 2023

(30) Foreign Application Priority Data
Dec. 15, 2020 (DK) PA 2020 01402

(51) Int. Cl. C07K 14/32; C12N 15/75; C12N 9/26; C12N 9/24; C12N 9/50; C12N 9/88

(52) U.S. Cl. CPC C07K 14/32; C12N 15/75; C12N 9/2411; C12N 9/248; C12N 9/50; C12N 9/88; C12Y 304/21062; C12Y 402/02012

(57) ABSTRACT

The present invention relates to mutated bacterial host cells, said host cells producing a polypeptide of interest and having at least one disrupted flagellum gene, and to nucleic acid constructs and vectors encoding at least one flagellum polypeptide with reduced or eliminated activity as well as to methods of producing one or more polypeptide of interest in said host cells.

### REFERENCE TO A SEQUENCE LISTING

This application contains a Sequence Listing in computer readable form, which is incorporated herein by reference.

### BACKGROUND OF THE INVENTION

### Field of the Invention

The present invention relates to mutated bacterial host cells, said host cells producing one or more polypeptide of interest and having one or more disrupted flagellum gene, and to nucleic acid constructs and vectors encoding at least one flagellum polypeptide with reduced or eliminated activity as well as to methods of producing one or more polypeptide of interest in said host cells.

### Description of the Related Art

Recombinant gene expression in recombinant host cells, such as bacterial host cells, is a common method for recombinant protein production. Recombinant proteins produced in prokaryotic systems are enzymes and other valuable proteins. In industrial and commercial purposes, the productivity of the applied cell systems, i.e. the production of total protein per fermentation unit, is an important factor of production costs. Traditionally, yield increases have been achieved through mutagenesis and screening for increased production of proteins of interest. However, this approach is mainly only useful for the overproduction of endogenous proteins in isolates containing the enzymes of interest. Therefore, for each new protein or enzyme product, a lengthy strain and process development program is required to achieve improved productivities.

For the overexpression of heterologous proteins in prokaryotic systems, the production process is recognized as a complex multi-phase and multi-component process. Cell growth and product formation are determined by a wide range of parameters, including the composition of the culture medium, fermentation pH, fermentation temperature, dissolved oxygen tension, shear stress, and bacterial morphology.

Various approaches to improve transcription have been used in bacteria. For the expression of heterologous genes, codon-optimized, synthetic genes can improve the transcription rate (WO9923211, Novozymes A/S). To obtain high-level expression of a particular gene, a well-established procedure is targeting multiple copies of the recombinant gene constructs to the locus of a highly expressed endogenous gene. A further strategy to increase protein yield is to reduce the proteolytic degradation of recombinant proteins by the disruption of native proteases as described in WO9629391 (Novozymes A/S). Despite the presented approaches, it is of continuous interest to further improve recombinant protein production in bacterial host cells.

The object of the present invention is to provide a modified bacterial host strain and a method of protein production with increased productivity or yield of recombinant protein.

### SUMMARY OF THE INVENTION

The present invention is based on the surprising and inventive finding that host cells with a reduced or eliminated expression of at least one flagellum (plural: flagella) gene, or with at least one mutated flagellum gene, results in improved expression, activity and/or yield of heterologous proteins compared to the expression of the same heterologous protein in host cells with native flagellum gene expression or non-mutated flagellum genes.

Flagella are long protein filaments of uniform length that are responsible for cell motility. Flagella consist of molecules of a globular protein, flagellin, that are aggregated in a helical chain. They are anchored in the plasma membrane and the number of flagella per cell may range from one to several hundred. By spinning around their axis in a corkscrew motion, flagella propel the cell, which motion is often a response to a chemical concentration gradient, indicating a sensory feedback regulation system, which is the basis for bacterial chemotaxis. The flagellum rotary device is considered to exclusively have evolved for bacterial locomotion. It is believed that more than 40 genes are involved in the construction of a flagellum, and an export apparatus specific for flagellum proteins, a scaffolding protein and capping proteins are elaborated for efficient construction.

Surprisingly, by reduced or eliminated expression of at least one flagellum gene, or by mutation or deletion of at least one flagellum gene, the present invention results in increased productivity and/or activity of recombinant protein, which has been shown herein for expression of amylase, protease, nattokinase, xylanase, and xanthan lyase molecules. As can be seen from the examples below, a bacterial host cell with a mutated or deleted flagellum gene, or with a reduced or eliminated flagellum gene expression, provides improved recombinant protein yield, such as an 2.76 fold increased recombinant amylase activity, 2.0 fold increase for nattokinase, 1.11 fold increase for AprH protease, 1.68 fold increase for Xylanase, and 1.32 fold increase for xanthan lyase.

Surprisingly, with the approach of targeting a single flagella gene, not only expression of the gene(s) targeted by the sgRNA(s) was efficiently inhibited, but also other flagella genes in the operon downstream and upstream of the sgRNA target site which were not directly deleted or targeted.

We expect that this finding also applies to other proteins, such as other proteins, enzymes and polypeptides with enzymatic activity. Based on the fact that the disruption of each flagellum gene targeted in the examples resulted in increased protein activity, it is expected that this finding also applies to the deletion/mutation/silencing of other flagellum genes than the flagellum genes targeted in the present disclosure.

Thus, in a first aspect the present invention relates to a mutated bacterial host cell comprising a heterologous promoter operably linked to a first heterologous polynucleotide encoding one or more polypeptide of interest, wherein expression of at least one flagellum gene is reduced or eliminated compared to a non-mutated otherwise isogenic or parent cell.

In a second aspect, the present invention relates to a method for producing one or more polypeptide of interest, the method comprising:
i) providing a bacterial host cell according to the first aspect,
ii) cultivating said host cell under conditions conducive for expression of the one or more polypeptide of interest; and
iii) optionally, recovering the one or more polypeptide of interest.

In a third aspect, the present invention relates to a nucleic acid construct comprising a polynucleotide encoding at least one flagellum polypeptide comprising or consisting of a polypeptide sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100% to any one of SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, and SEQ ID NO: 63.

In a final and fourth aspect, the present invention also relates to an expression vector comprising a nucleic acid construct according to the third aspect of the invention.

### BRIEF DESCRIPTION OF THE FIGURES

FIG. 1 shows a swimming/motility assay of host cells with impaired flagellum gene expression.

FIG. 2 shows amylase activity data of cultivated host cells with impaired flagellum gene expression.

FIG. 3 shows (A) swimming/motility assay and (B) amylase activity data of host cells with a deleted flagellum gene.

FIG. 4 shows targeted flagella genes of the flagella operon.

FIG. 5 shows effect on steady-state RNA levels of the flagella operon when targeted with CISP at different locations.

FIG. 6 shows effect on steady-state RNA levels of the flagella operon when the flgE gene is deleted by a marker gene.

FIG. 7 shows increased recombinant protein production for host cells with a deleted flagellum gene.

### DEFINITIONS

In accordance with this detailed description, the following definitions apply. Note that the singular forms “a,” “an,” and “the” include plural references unless the context clearly dictates otherwise.

Reference to “about” a value or parameter herein includes aspects that are directed to that value or parameter per se. For example, description referring to “about X” includes the aspect “X”.

Unless defined otherwise or clearly indicated by context, all technical and scientific terms used herein have the same meaning as commonly understood by one of ordinary skill in the art to which this invention belongs.
Amylase: The term amylase, JE1 amylase, or JE1 means a glycosylase (EC 3.2), more specifically a glycosidase (EC 3.2.1) or an alpha-amylase (EC 3.2.1.1) that catalyzes the endohydrolysis of (1→4)-alpha-D-glucosidic linkages in polysaccharides containing three or more (1→4)-alpha-linked D-glucose units. For purposes of the present invention, amylase activity is determined according to the procedure described in the Examples. In one aspect, the polypeptides of the present invention have at least 20%, e.g., at least 40%, at least 50%, at least 60%, at least 70%, at least 80%, at least 90%, at least 95%, or at least 100% of the amylase activity of the mature polypeptide of SEQ ID NO: 8.
Catalytically inactive site-directed polypeptide: The term “catalytically inactive site-directed polypeptide” or CISP means a site-directed polypeptide with decreased or inactivated nuclease activity, wherein the CISP is directed towards an RNA-sequence or DNA-sequence. Non-limiting examples for catalytically inactive site-directed polypeptides are a catalytically inactive CRISPR-associated protein (such as a catalytically inactive MAD7/Cas12a endonuclease), a catalytically inactive zinc finger nuclease (ZFN), a zinc finger, a catalytically inactive transcription activator-like effector nuclease (TALEN), a TALE, and a catalytically inactive meganuclease. The site specificity of a CISP is determined by the polypeptide sequence of the CISP, e.g. for ZFN, TALEN, TALE or meganuclease; or the site specificity of a CISP is determined by a guide RNA, such as a single guide RNA for a CRISPR/Cas nuclease, e.g. the CRISPR/Cas12a nuclease MAD7. Thereby the CISP is directed towards a target RNA-sequence or target DNA-sequence to sterically block the transcription of the target gene by the RNA-polymerase or to prevent the translation of the target mRNA by the ribosomal subunits, respectively.
Catalytic domain: The term “catalytic domain” means the region of an enzyme containing the catalytic machinery of the enzyme.
cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.
Coding sequence: The term “coding sequence” means a polynucleotide, which directly specifies the amino acid sequence of a polypeptide. The boundaries of the coding sequence are generally determined by an open reading frame, which begins with a start codon, such as ATG, GTG, or TTG, and ends with a stop codon, such as TAA, TAG, or TGA. The coding sequence may be a genomic DNA, cDNA, synthetic DNA, or a combination thereof.
Control sequences: The term “control sequences” means nucleic acid sequences necessary for expression of a polynucleotide encoding a polypeptide of the present invention. Each control sequence may be native (i.e., from the same gene) or heterologous (i.e., from a different gene) to the polynucleotide encoding the polypeptide or native or heterologous to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.
Expression: The term “expression” means any step involved in the production of a polypeptide including, but not limited to, transcription, post-transcriptional modification, translation, post-translational modification, and secretion.
Expression vector: The term “expression vector” means a linear or circular DNA molecule that comprises a polynucleotide encoding a polypeptide and is operably linked to control sequences that provide for its expression.
Flagellum: The term flagellum (plural: flagella) describes a rotary device that has evolved exclusively for bacterial locomotion. It rotates at several hundred revolutions per second. More than 40 genes are involved in the construction of a flagellum. The number of flagella, helical handedness of filament and rotational direction of the flagellar motor determine the behavioral range of each bacterial species.
Flagellum activity: The term flagella activity or flagellum activity describes the activity of intact flagella or flagellum of a host cell. Flagella activity includes the cell's capability to perform cell motility, such as cell motility in liquid (swimming motility) or cell motility on solid surfaces. Examples of solid surface motility are swarming motility, gliding motility and twitching motility. Flagella activity is dependent on the expression, assembly and functionality of a plurality of flagellum sub-units (flagellum polypeptides) encoded by more than 40 genes. Gene disruption, gene deletion, gene mutation or decreased gene expression of one or more of the flagellum genes can lead to impaired and decreased flagella activity. Overexpression of the MotI protein can further be used to inhibit flagella activity.
Flagellum gene: The term flagellum gene describes a polynucleotide sequence encoding a flagellum polypeptide which is comprised in the flagellum complex and also includes flagellum associated regulatory genes of the flagellum operon. Non-limiting examples for a flagellum gene are the genes flgA, flgB, flgC, flgD, flgE, flgF, flgG, flgH, flgI, flgJ, flgK, flgL, flgM, flgN, flhA, flhB, flhC, flhD, flhE, flhF, flhG, flhO, flhP, fliA, fliD, fliE, fliF, fliG, fliH, fliI, fliJ, fliK, fliL, fliM, fliN, fliO, fliP, fliQ, fliR, fliS, fliT, fliY, fliZ, hag, motA, motB, ylxF, swrD, cheY, cheB, cheA, cheVV, cheC, cheD, sigD, and swrB, such as the polynucleotide sequences with SEQ ID NO: 15 (flgE), SEQ ID NO: 16 (fliR) and SEQ ID NO: 17 (flhG). Non-limiting examples of flagellum polypeptides which are encoded by a flagellum gene are the flagella polypeptides FlgA, FlgB, FlgC, FlgD, FlgE, FlgF, FlgG, FlgH, FlgI, FlgJ, FlgK, FlgL, FlgM, FlgN, FlhA, FlhB, FlhC, FlhD, FlhE, FlhF, FlhG, FlhO, FlhP, FliA, FliD, FliE, FliF, FliG, FliH, FliI, FliJ, FliK, FliL, FliM, FliN, FliO, FliP, FliQ, FliR, FliS, FliT, FliY, FliZ, Hag, MotA, MotB, MotI, YlxF, SwrD, CheY, CheB, CheA, CheW, CheC, CheD, SigD, and SwrB. In one aspect, the flagella genes of the present invention encode a flagellum polypeptide having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to a polypeptide sequence selected from the list of SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63 and SEQ ID NO: 71.
Flagellum polypeptide: The term flagellum polypeptide means a polypeptide which is comprised in the flagellum or flagella, or a polypeptide which has a regulatory function within the flagellum operon, wherein the flagellum polypeptide is encoded by a flagellum gene such as a flagellum gene described above. Non-limiting examples of flagella polypeptides are the flagella polypeptides FlgA, FlB, FlgC, FlgD, FlgE, FlgF, FlgG, FlgH, FlgI, FlgJ, FlgK, FlgL, FlgM, FlgN, FlhA, FlhB, FlhC, FlhD, FlhE, FlhF, FlhG, FlhO, FlhP, FliA, FliD, FliE, FliF, FliG, FliH, FliI, FliJ, FliK, FliL, FliM, FliN, FliO, FliP, FliQ, FliR, FliS, FliT, FliY, FliZ, Hag, MotA, MotB, MotI, YlxF, SwrD, CheY, CheB, CheA, CheW, CheC, CheD, SigD, and SwrB. In one aspect, the flagella polypeptides of the present invention have at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to a polypeptide sequence selected from the list of SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63 and SEQ ID NO: 71.
Fragment: The term “fragment” means a flagellum polypeptide sub-unit, or a flagellum complex sub-unit having one or more (e.g., several) amino acids absent from the amino and/or carboxyl terminus of a mature polypeptide or domain; wherein the fragment has no flagella activity or functionality.
Fusion polypeptide: The term “fusion polypeptide” is a polypeptide in which one polypeptide is fused at the N-terminus or the C-terminus of a polypeptide of the present invention. A fusion polypeptide is produced by fusing a polynucleotide encoding another polypeptide to a polynucleotide of the present invention. Techniques for producing fusion polypeptides are known in the art and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779). A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 263: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.
Heterologous: The term “heterologous” means, with respect to a host cell, that a polypeptide or nucleic acid does not naturally occur in the host cell. The term “heterologous” means, with respect to a polypeptide or nucleic acid, that a control sequence, e.g., promoter, or domain of a polypeptide or nucleic acid is not naturally associated with the polypeptide or nucleic acid, i.e., the control sequence is from a gene other than the gene encoding the mature polypeptide of SEQ ID NO: 8.
Host cell: The term “host cell” means any microbial or plant cell into which a nucleic acid construct or expression vector comprising a polynucleotide of the present invention has been introduced. Methods for introduction include but are not limited to protoplast fusion, transfection, transformation, electroporation, conjugation, and transduction. In some embodiments, the host cell is an isolated recombinant host cell that is partially or completely separated from at least one other component with, including but not limited to, proteins, nucleic acids, cells, etc.
Hybrid polypeptide: The term “hybrid polypeptide” means a polypeptide comprising domains from two or more polypeptides, e.g., a binding module from one polypeptide and a catalytic domain from another polypeptide. The domains may be fused at the N-terminus or the C-terminus.
Hybridization: The term “hybridization” means the pairing of substantially complementary strands of nucleic acids, using standard Southern blotting procedures. Hybridization may be performed under medium, medium-high, high or very high stringency conditions. Medium stringency conditions means prehybridization and hybridization at 42° C. in 5× SSPE, 0.3% SDS, 200 micrograms/ml sheared and denatured salmon sperm DNA, and 35% formamide for 12 to 24 hours, followed by washing three times each for 15 minutes using 0.2× SSC, 0.2% SDS at 55° C. Medium-high stringency conditions means prehybridization and hybridization at 42° C. in 5× SSPE, 0.3% SDS, 200 micrograms/ml sheared and denatured salmon sperm DNA, and 35% formamide for 12 to 24 hours, followed by washing three times each for 15 minutes using 0.2× SSC, 0.2% SDS at 60° C. High stringency conditions means prehybridization and hybridization at 42° C. in 5× SSPE, 0.3% SDS, 200 micrograms/ml sheared and denatured salmon sperm DNA, and 50% formamide for 12 to 24 hours, followed by washing three times each for 15 minutes using 0.2× SSC, 0.2% SDS at 65° C. Very high stringency conditions means prehybridization and hybridization at 42° C. in 5× SSPE, 0.3% SDS, 200 micrograms/ml sheared and denatured salmon sperm DNA, and 50% formamide for 12 to 24 hours, followed by washing three times each for 15 minutes using 0.2× SSC, 0.2% SDS at 70° C.
Isolated: The term “isolated” means a polypeptide, nucleic acid, cell, or other specified material or component that is separated from at least one other material or component with which it is naturally associated as found in nature, including but not limited to, for example, other proteins, nucleic acids, cells, etc. An isolated polypeptide includes, but is not limited to, a culture broth containing the secreted polypeptide.
Mature polypeptide: The term “mature polypeptide” means a polypeptide in its mature form following N-terminal processing (e.g., removal of signal peptide). In one aspect, the mature polypeptide is SEQ ID NO: 8. In another aspect the polypeptide is the mature polypeptide of any one of amino acid sequences with SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63 and SEQ ID NO: 71.
Mature polypeptide coding sequence: The term “mature polypeptide coding sequence” means a polynucleotide that encodes a mature polypeptide having amylase activity or a mature polypeptide being comprised in the flagellum complex and having flagella activity or flagella functionality.
Native: The term “native” means a nucleic acid or polypeptide naturally occurring in a host cell.
Non-coding RNA: The term non-coding RNA (ncRNA) or non-coding RNA molecule means an RNA molecule that is not translated into a protein. Abundant and functionally important types of ncRNA include tRNAs, rRNAs, as well as small RNAs such as microRNAs, siRNAs, piRNAs, snoRNAs, snRNAs, exRNAs, scaRNAs, lncRNAs, or synthetic ncRNAs. In general, ncRNAs function to regulate gene expression at the transcriptional and post-transcriptional level, or assist an endonuclease, such as a LISP, to target a DNA or RNA sequence. ncRNAs can therefore be designed to reduce or eliminate the expression of a target gene. Additionally or alternatively, ncRNA expression can be reduced or depleted to increase the expression of certain genes. An example for ncRNA is a guide RNA of a CRISPR/Cas endonuclease.
Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.
Operably linked: The term “operably linked” means a configuration in which a control sequence is placed at an appropriate position relative to the coding sequence of a polynucleotide such that the control sequence directs expression of the coding sequence.
Protease: The term “nattokinase” or “protease” means a protease (EC 3.4.21.62) that catalyzes the hydrolysis of proteins with broad specificity for peptide bonds, and a preference for a large uncharged residue in P1. A protease or nattokinase conducts proteolysis by hydrolysis of the peptide bonds that link amino acids together in the polypeptide chain forming the protein. For purposes of the present invention, protease/nattokinase activity is determined according to the procedure described in the Examples. In one aspect, the polypeptides of the present invention have at least 20%, e.g., at least 40%, at least 50%, at least 60%, at least 70%, at least 80%, at least 90%, at least 95%, or at least 100% of the protease activity of the mature polypeptide of SEQ ID NO: 72. In another aspect, the polypeptides of the present invention have at least 20%, e.g., at least 40%, at least 50%, at least 60%, at least 70%, at least 80%, at least 90%, at least 95%, or at least 100% of the nattokinase activity of the mature polypeptide of SEQ ID NO: 73.
Purified: The term “purified” means a nucleic acid or polypeptide that is substantially free from other components as determined by analytical techniques well known in the art (e.g., a purified polypeptide or nucleic acid may form a discrete band in an electrophoretic gel, chromatographic eluate, and/or a media subjected to density gradient centrifugation). A purified nucleic acid or polypeptide is at least about 50% pure, usually at least about 60%, about 65%, about 70%, about 75%, about 80%, about 85%, about 90%, about 91%, about 92%, about 93%, about 94%, about 95%, about 96%, about 97%, about 98%, about 99%, about 99.5%, about 99.6%, about 99.7%, about 99.8% or more pure (e.g., percent by weight on a molar basis). In a related sense, a composition is enriched for a molecule when there is a substantial increase in the concentration of the molecule after application of a purification or enrichment technique. The term “enriched” refers to a compound, polypeptide, cell, nucleic acid, amino acid, or other specified material or component that is present in a composition at a relative or absolute concentration that is higher than a starting composition.
Recombinant: The term “recombinant,” when used in reference to a cell, nucleic acid, protein or vector, means that it has been modified from its native state. Thus, for example, recombinant cells express genes that are not found within the native (non-recombinant) form of the cell, or express native genes at different levels or under different conditions than found in nature. Recombinant nucleic acids differ from a native sequence by one or more nucleotides and/or are operably linked to heterologous sequences, e.g., a heterologous promoter in an expression vector. Recombinant proteins may differ from a native sequence by one or more amino acids and/or are fused with heterologous sequences. A vector comprising a nucleic acid encoding a polypeptide is a recombinant vector. The term “recombinant” is synonymous with “genetically modified” and “transgenic”.
Heteroduplex: The term heteroduplex means a double-stranded nucleic acid molecule consisting of a DNA molecule and a RNA molecule (RNA:DNA heteroduplex), or consisting of two RNA molecules (RNA:RNA heteroduplex). The heteroduplex can be free of nucleotide mismatches (non-complementary) between the nucleic acid sequences or the heteroduplex can comprise at least one nucleotide mismatch, or a region of nucleotide mismatches. In vivo, these heteroduplexes can result from mutation, genetic recombination, gene silencing by ncRNAs or by targeting selected genes or their transcripts with programmed nucleases, such as a LISP. Within the context of the present invention a RNA:DNA heteroduplex comprises a DNA region of a targeted gene sequence and a non-coding RNA molecule configured to bind or hybridize with said DNA target sequence. Within the context of the present invention a RNA:RNA heteroduplex comprises a mRNA molecule, transcribed from a target gene, and a non-coding RNA molecule configured to bind or hybridize said target mRNA molecule.
RNA transcript: The term RNA transcript means a messenger RNA or mRNA transcribed from a gene sequence by the RNA-polymerase. Non-limiting examples for a RNA transcript are the mRNA molecules transcribed from the flagellum genes flgA, flgB, flgC, flgD, flgE, flgF, flgG, flgH, flgI, flgJ, flgK, flgL, flgM, flgN, flhA, flhB, flhC, flhD, flhE, flhF, flhG, flhO, flhP, fliA, fliD, fliE, fliF, fliG, fliH, fliI, fliJ, fliK, fliL, fliM, fliN, fliO, fliP, fliQ, fliR, fliS, fliT, fliY, fliZ, hag, motA, motB, motI, ylxF, swrD, cheY, cheB, cheA, cheVV, cheC, cheD, sigD, and swrB.
Sequence identity: The relatedness between two amino acid sequences or between two nucleotide sequences is described by the parameter “sequence identity”.

For purposes of the present invention, the sequence identity between two amino acid sequences is determined as the output of “longest identity” using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 6.6.0 or later. The parameters used are a gap open penalty of 10, a gap extension penalty of 0.5, and the EBLOSUM62 (EMBOSS version of BLOSUM62) substitution matrix. In order for the Needle program to report the longest identity, the -nobrief option must be specified in the command line. The output of Needle labeled “longest identity” is calculated as follows:

(Identical Residues×100)/(Length of Alignment−Total Number of Gaps in Alignment).

For purposes of the present invention, the sequence identity between two polynucleotide sequences is determined as the output of “longest identity” using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 6.6.0 or later. The parameters used are a gap open penalty of 10, a gap extension penalty of 0.5, and the EDNAFULL (EMBOSS version of NCBI NUC4.4) substitution matrix. In order for the Needle program to report the longest identity, the nobrief option must be specified in the command line. The output of Needle labeled “longest identity” is calculated as follows:

(Identical Deoxyribonucleotides×100)/(Length of Alignment−Total Number of Gaps in Alignment)
Guide RNA: The term single guide RNA, guide RNA or ncRNA means a RNA sequence of a RNA molecule that binds to a gene target or transcript target, to form a DNA:RNA heteroduplex or a RNA:RNA heteroduplex, and thereby facilitates binding of a CISP, such as a CRISPR/Cas 12a nuclease or an inactivated nuclease to said heteroduplex. To perform this function efficiently, the ncRNA comprises a first sequence being specific for the target sequence, and a second sequence specific for the nuclease and facilitating the binding of the nuclease to the ncRNA. Within the context of the present invention the guide RNA is categorized as a ncRNA. In one embodiment the guide RNA is complementary to any one of nucleotide sequences with SEQ ID NO: 18 to 23, or the guide RNA comprises any one of nucleotide sequences with SEQ ID NO: 18 to 23.
Subsequence: The term “subsequence” means a polynucleotide having one or more (e.g., several) nucleotides absent from the 5′ and/or 3′ end of a mature polypeptide coding sequence; wherein the subsequence encodes a fragment comprised in the flagellum complex.
Target polynucleotide sequence: The term “target polynucleotide sequence” means a polynucleotide sequence of a target gene or a polynucleotide sequence of an RNA transcript of said target gene. Each target gene, such as a flagellum gene, can facilitate one or several target polynucleotide sequences. Within the context of the present invention, the target polynucleotide sequence is subject to binding by or hybridization with a ncRNA or CISP. Additionally or alternatively, the target polynucleotide sequence comprises, consists of, or consists essentially of the entire coding region of a flagellum gene, e.g. when the flagellum gene or the coding region thereof is deleted either fully or partially. Additionally or alternatively, the target polynucleotide sequence is located adjacent a flagellum target gene, facilitating the deletion of the target gene by homologous recombination of the flanking DNA regions, thereby enabling the deletion of the flagellum gene or the coding region thereof either partially or fully. In one embodiment the target polynucleotide sequence comprises one of the nucleotide sequences with SEQ ID NO: 18 to 23.
Variant: The term “variant” means a polypeptide having amylase activity or a polypeptide contributing to reduced or eliminated flagellum activity, said variant comprising a man-made mutation, i.e., a substitution, insertion, and/or deletion (e.g., truncation), at one or more (e.g., several) positions. A substitution means replacement of the amino acid occupying a position with a different amino acid; a deletion means removal of the amino acid occupying a position; and an insertion means adding one or more (e.g. several) amino acids, e.g. 1-5 amino acids, adjacent to the amino acid occupying a position.
Wild-type: The term “wild-type” in reference to an amino acid sequence or nucleic acid sequence means that the amino acid sequence or nucleic acid sequence is a native or naturally-occurring sequence. As used herein, the term “naturally-occurring” refers to anything (e.g., proteins, amino acids, or nucleic acid sequences) that is found in nature. Conversely, the term “non-naturally occurring” refers to anything that is not found in nature (e.g., recombinant nucleic acids and protein sequences produced in the laboratory or modification of the wild-type sequence).
Xanthan lyase: The term “xanthan lyase” means a xanthan lyase, a xanthan-degrading enzyme (EC 4.2.2.12) that catalyses cleavage of the terminal beta-D-mannosyl-(1->4)-beta-D-glucuronosyl linkage of the side-chain of the polysaccharide xanthan, leaving a 4-deoxy-alpha-L-threo-hex-4-enuronosyl group at the terminus of the side-chain. Xanthan lyases release pyruvylated mannose via eliminate cleavage with concomitant production of a Δ4,5-unsaturated glucuronic acid (Δ4,5-ene-GlcA) side chain. For purposes of the present invention, xanthan lyase activity is determined according to the procedure described in the Examples. In one aspect, the polypeptides of the present invention have at least 20%, e.g., at least 40%, at least 50%, at least 60%, at least 70%, at least 80%, at least 90%, at least 95%, or at least 100% of the xanthan lyase activity of the mature polypeptide of SEQ ID NO: 75.
Xylanase: The term “xylanase” means an endo-1,4-beta-xylanase (EC 3.2.1.8), such as an endo-1,4-beta-xylanhydrolase, that catalyzes endohydrolysis of (1->4)-beta-D-xylosidic linkages in xylans. For purposes of the present invention, xylanase activity is determined according to the procedure described in the Examples. In one aspect, the polypeptides of the present invention have at least 20%, e.g., at least 40%, at least 50%, at least 60%, at least 70%, at least 80%, at least 90%, at least 95%, or at least 100% of the xylanase activity of the mature polypeptide of SEQ ID NO: 74.

### DETAILED DESCRIPTION OF THE INVENTION

### Host Cells

In a first aspect, the invention relates to a mutated bacterial host cell comprising a heterologous promoter operably linked to one or more first heterologous polynucleotide encoding a polypeptide of interest, wherein expression of at least one flagellum gene is reduced or eliminated compared to a non-mutated otherwise isogenic cell or parent cell. Additionally or alternatively the expression of the at least one flagellum gene is similar to the non-mutated otherwise isogenic or parent cell, wherein the polynucleotide sequence of the at least one flagellum gene is altered compared to the non-mutated otherwise isogenic cell or parent cell, said alteration being selected from the list of a premature stop-codon, a nucleotide insertion, and a nucleotide deletion, such as the deletion of one or more nucleotides of the polynucleotide sequence of the flagella gene.

A construct or vector comprising a polynucleotide is introduced into a host cell so that the construct or vector is maintained as a chromosomal integrant or as a self-replicating extra-chromosomal vector as described earlier. The choice of a host cell will to a large extent depend upon the gene encoding the polypeptide and its source.

In one embodiment the flagellum gene encodes a flagellum polypeptide selected from the list of FlgA, FlgB, FlgC, FlgD, FlgE, FlgF, FlgG, FlgH, FlgI, FlgJ, FlgK, FlgL, FlgM, FlgN, FlhA, FlhB, FlhC, FlhD, FlhE, FlhF, FlhG, FlhO, FlhP, FliA, FliD, FliE, FliF, FliG, FliH, FliI, FliJ, FliK, FliL, FliM, FliN, FliO, FliP, FliQ, FliR, FliS, FliT, FliY, FliZ, Hag, MotA, MotB, YlxF, SwrD, CheY, CheB, CheA, CheW, CheC, CheD, SigD, and/or SwrB; preferably the flagellum gene encodes the flagellum polypeptide FlgE, FliR and/or FlhG. The natural expression of the flagellum polypeptide may vary from species to species. Therefore, the selection of the targeted flagellum gene can be adapted to the flagellum gene expression profile of a certain host cell species. Preferably the reduced or eliminated expression of a single flagellum polypeptide results in a reduced or eliminated flagella activity of the host cell. Alternatively, the simultaneous reduced or eliminated expression of two, three or four flagella polypeptides results in a reduced or eliminated flagella activity. Additionally or alternatively, reduced or eliminated flagella activity is achieved by the overexpression of a motI gene encoding a MotI polypeptide with at least 60% sequence similarity to SEQ ID NO: 71.

In one embodiment the at least one flagellum gene is selected from the list of flgA, flgB, flgC, flgD, flgE, flgF, flgG, flgH, flgI, flgJ, flgK, flgL, flgM, flgN, flhA, flhB, flhC, flhD, flhE, flhF, flhG, flhO, flhP, fliA, fliD, fliE, fliF, fliG, fliH, fliI, fliJ, fliL, fliM, fliN, fliO, fliP, fliQ, fliR, fliS, fliT, fliY, fliZ, hag, motA, motB, ylxF, swrD, cheY, cheB, cheA, cheW, cheC, cheD, sigD, and/or swrB; preferably the at least one flagellum gene is flgE, fliR and/or flhG.

The expression of one or more of the flagella genes can be reduced or eliminated, such as a reduced or eliminated expression of two, three, four, five, six, seven, eight, or more than eight flagella genes, independently chosen from one another.

In one embodiment, the expression of the flagellum gene flgE is reduced or eliminated in combination with a reduced or eliminated expression of the flagellum gene fliR.

In one embodiment, the expression of the flagellum gene flgE is reduced or eliminated in combination with a reduced or eliminated expression of the flagellum gene flhG.

In one embodiment, the expression of the flagellum gene flhG is reduced or eliminated in combination with a reduced or eliminated expression of the flagellum gene fliR.

In one embodiment, the expression of the flagellum gene flgE is reduced or eliminated in combination with a reduced or eliminated expression of the flagellum gene fliR, and with a reduced or eliminated expression of the flagellum gene flhG.

In another embodiment, the expression of the at least one flagellum gene flgE, fliR and/or flhG is reduced or eliminated in combination with a reduced or eliminated expression of at least one flagellum gene selected from the list of flgA, flgB, flgC, flgD, flgF, flgG, flgH, flgI, flgJ, flgK, flgL, flgM, flgN, flhA, flhB, flhC, flhD, flhE, flhF, flhO, flhP, fliA, fliD, fliE, fliF, fliG, fliI, fliJ, fliK, fliL, fliM, fliN, fliO, fliP, fliQ, fliS, fliY, fliZ, hag, motA, motB, ylxF, swrD, cheY, cheB, cheA, cheW, cheC, cheD, sigD, and/or swrB.

In yet another embodiment the expression of two, three, four, or more than four flagella genes is reduced or eliminated, wherein the flagellum gene is selected from the list of flgA, flgB, flgC, flgD, flgE, flgF, flgG, flgH, flgI, flgJ, flgK, flgL, flgM, flgN, flhA, flhB, flhC, flhD, flhE, flhF, flhG, flhO, flhP, fliA, fliD, fluE, fliF, fliG, fliH, fliI, fliJ, fliK, fliL, fliM, fliN, fliO, fliP, fliQ, fliR, fliS, fliT, fliY, fliZ, hag, motA, motB, ylxF, swrD, cheY, cheB, cheA, cheW, cheC, cheD, sigD, and/or swrB.

In one embodiment, the at least one flagellum gene comprises or consists of a polynucleotide sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to a polynucleotide sequence selected from the list of SEQ ID NO: 15, SEQ ID NO: 16 and SEQ ID NO: 17.

In one embodiment the at least one flagellum gene comprises a polynucleotide sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to a polynucleotide sequence selected from the list of SEQ ID NO: 18 (flgE), SEQ ID NO: 19 (flgE), SEQ ID NO: 20 (fliR), SEQ ID NO: 21 (fliR), SEQ ID NO: 22 (flhG) and SEQ ID NO: 23 (flhG).

In another embodiment the at least one flagellum gene encodes a flagellum polypeptide comprising or consisting of an amino acid sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to a polypeptide sequence selected from the list of SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, and/or SEQ ID NO: 63.

In one embodiment the polynucleotide sequence of the at least one flagellum gene comprises at least one alteration selected from the list of a premature stop-codon, a nucleotide insertion, and a nucleotide deletion, such as the deletion of one or more nucleotides of the polynucleotide sequence of the flagella gene or its coding region, or the deletion of substantially all nucleotides of the polynucleotide sequence of the at least one flagellum gene or its coding region; or wherein the polynucleotide sequence of the at least one flagellum gene or its coding region is deleted in its entirety. The deletion of at least one flagellum gene can be achieved by substituting the open reading frame of the flagellum gene with another polynucleotide sequence, such as a polynucleotide sequence encoding a selection marker. In a preferred embodiment, the at least one flagellum gene is deleted by homologous recombination.

In one embodiment, the at least one alteration of the at least one flagellum gene results in a flagellum gene polynucleotide sequence comprising or consisting of a polynucleotide sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100% to a polynucleotide sequence selected from the list of SEQ ID NO: 15, SEQ ID NO: 16 and SEQ ID NO: 17.

In another embodiment, the at least one alteration of the at least one flagellum gene results in a flagellum polypeptide sequence comprising or consisting of a polypeptide sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100% to a polypeptide sequence selected from the list of SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, and SEQ ID NO: 63.

In a further embodiment the host cell comprises a reduced or eliminated flagella activity, a reduced or eliminated cell motility, or a reduced or eliminated swimming activity. The reduced or eliminated cell motility is identified by comparing the mutant cell motility to the cell motility of a non-mutated parent cell, and is an indication for a reduced or eliminated flagella activity and/or a reduced or eliminated flagellum gene expression.

In one embodiment the cell motility of the mutated host cell is reduced by at least 10%, e.g., at least 15%, at least 20%, at least 30%, at least 40%, at least 50%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or by 100% compared to a non-mutated otherwise isogenic or parent cell.

In a preferred embodiment the host cell is a Gram-negative bacteria selected from the group consisting of Campylobacter, E. coli, Flavobacterium, Fusobacterium, Helicobacter, llyobacter, Neisseria, Pseudomonas, Salmonella, and Ureaplasma cells, or the host cell is a Gram-positive cell selected from the group consisting of Bacillus, Clostridium, Enterococcus, Geobacillus, Lactobacillus, Lactococcus, Oceanobacillus, Staphylococcus, Streptococcus, or Streptomyces cells, such as Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, Bacillus thuringiensis, Streptococcus equisimilis, Streptococcus pyogenes, Streptococcus uberis, and Streptococcus equi subsp. zooepidemicus, Streptomyces achromogenes, Streptomyces avermitilis, Streptomyces coelicolor, Streptomyces griseus, and Streptomyces lividans cells, preferably the host cell is a Bacillus cell, most preferably a Bacillus subtilis or a Bacillus licheniformis cell.

In a preferred embodiment the host cell is a Bacillus subtilis cell.

In another preferred embodiment the host cell is a Bacillus licheniformis cell.

In another embodiment, the one or more polypeptide of interest comprises an enzyme; preferably the enzyme is selected from the group consisting of hydrolase, isomerase, ligase, lyase, oxidoreductase, or transferase; more preferably an aminopeptidase, amylase, carbohydrase, carboxypeptidase, catalase, cel lobiohydrolase, cellulase, chitinase, cutinase, cyclodextrin glycosyltransferase, deoxyribonuclease, endoglucanase, esterase, alpha-galactosidase, beta-galactosidase, alpha-glucosidase, beta-glucosidase, invertase, laccase, lipase, mannosidase, mutanase, nuclease, oxidase, pectinolytic enzyme, peroxidase, phosphodiesterase, phytase, polyphenoloxidase, proteolytic enzyme, ribonuclease, transglutaminase, xylanase, and beta-xylosidase; even more preferably the polypeptide of interest comprises an amylase.

In one embodiment, the one or more polypeptide of interest comprises an amylase, such as an amylase which comprises, consists essentially of, or consists of the mature polypeptide having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the amino acid sequence of SEQ ID NO: 8.

In one embodiment the one or more polypeptide of interest comprises, consists of, or essentially consists of a protease.

In one embodiment, the one or more polypeptide of interest comprises a protease, such as a protease which comprises, consists essentially of, or consists of the mature polypeptide having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the amino acid sequence of SEQ ID NO: 72.

In another embodiment the one or more polypeptide of interest comprises, consists of, or essentially consists of a phosphodiesterase (PDE).

In another embodiment, the one or more polypeptide of interest comprises, consists of, or essentially consists of a nattokinase.

In one embodiment, the one or more polypeptide of interest comprises a nattokinase, such as a nattokinase which comprises, consists essentially of, or consists of the mature polypeptide having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the amino acid sequence of SEQ ID NO: 73.

In another embodiment, the one or more polypeptide of interest comprises, consists of, or essentially consists of a xylanase.

In one embodiment, the one or more polypeptide of interest comprises a xylanase, such as a xylanase which comprises, consists essentially of, or consists of the mature polypeptide having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the amino acid sequence of SEQ ID NO: 74.

In another embodiment, the one or more polypeptide of interest comprises, consists of, or essentially consists of a xanthan lyase.

In one embodiment, the one or more polypeptide of interest comprises a xanthan lyase, such as a xanthan lyase which comprises, consists essentially of, or consists of the mature polypeptide having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the amino acid sequence of SEQ ID NO: 75.

In some embodiments, the one or more polypeptide is heterologous to the recombinant host cell.

In some embodiments, at least one of the one or more control sequences is heterologous to the polynucleotide encoding the one or more polypeptide.

In some embodiments, the recombinant host cell comprises at least two copies, e.g., three, four, or five, of the polynucleotide of the present invention.

In one embodiment, the expression of the polypeptide of interest is increased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 100%, at least 105%, at least 110%, at least 115%, at least 120%, at least 125%, at least 130%, at least 135%, at least 140%, at least 145%, at least 150%, at least 155%, at least 160%, at least 165%, at least 170%, at least 175%, at least 180%, at least 185%, at least 190%, at least 195%, at least 200%, at least 205%, at least 210%, at least 215%, at least 220%, at least 225%, at least 230%, at least 235%, at least 240%, at least 245%, at least 250%, at least 255%, at least 260%, at least 265%, at least 270%, at least 275%, at least 280%, at least 285%, at least 290%, at least 295%, at least 300%, at least 305%, at least 310%, at least 315%, at least 320%, at least 325%, at least 330%, at least 335%, at least 340%, at least 345%, at least 350%, at least 355%, at least 360%, at least 365%, at least 370%, at least 375%, at least 380%, at least 385%, at least 390%, at least 395%, or at least 400%, relative to the expression of the polypeptide of interest in a parent host cell which does not comprises a reduced or eliminated flagella activity, when cultivated under identical conditions. Preferably, the parent host cell is otherwise isogenic to the host cell according to the first aspect.

In one embodiment, the expression of the polypeptide of interest is increased when the cell is cultivated in a batch fermentation mode.

In one embodiment, the expression of the polypeptide of interest is increased when the cell is cultivated in a fed-batch fermentation mode.

In one embodiment the increased expression of the polypeptide of interest is achieved after 24 hours, 48 hours, 72 hours, 96 hours, or 120 hours of cultivation.

In one embodiment the increased expression of the polypeptide of interest is achieved after 24 hours, or after at least 24 hours of cultivation, preferably the cultivation is a batch mode.

In one embodiment the increased expression of the polypeptide of interest is achieved after 120 hours, or after at least 120 hours of cultivation, preferably the cultivation is a fed-batch mode.

In one embodiment, the polypeptide of interest is a protease, and the expression of the protease is increased up to 11%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90(Yo, up to 100%, up to 105%, up to 110%, up to 115%, up to 120%, up to 125%, up to 130%, up to 135%, up to 140%, up to 145%, up to 150%, up to 155%, up to 160%, up to 165%, up to 170%, up to 175%, up to 180%, up to 185%, up to 190%, up to 195%, up to 200%, up to 205%, up to 210%, up to 215%, up to 220%, up to 225%, up to 230%, up to 235%, up to 240%, up to 245%, up to 250%, up to 255%, up to 260%, up to 265%, up to 270%, up to 275%, up to 280%, up to 285%, up to 290%, up to 295%, up to 300%, up to 305%, up to 310%, up to 315%, up to 320%, up to 325%, up to 330%, up to 335%, up to 340%, up to 345%, up to 350%, up to 355%, up to 360%, up to 365%, up to 370%, up to 375%, up to 380%, up to 385%, up to 390%, up to 395%, or up to 400%, preferably up to 11%, relative to the expression of the polypeptide of interest in a parent host cell which does not comprises a reduced or eliminated flagella activity, when cultivated under identical conditions. Preferably, the parent host cell is otherwise isogenic to the host cell according to the first aspect.

In one embodiment, the polypeptide of interest is a nattokinase, and the expression of the nattokinase is increased up to 4%, up to 5%, up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 100%, up to 104%, up to 105%, up to 110%, up to 115%, up to 120%, up to 125%, up to 130%, up to 135%, up to 140%, up to 145%, up to 150%, up to 155%, up to 160%, up to 165%, up to 170%, up to 175%, up to 180%, up to 185%, up to 190%, up to 195%, up to 200%, up to 205%, up to 210%, up to 215%, up to 220%, up to 225%, up to 230%, up to 235%, up to 240%, up to 245%, up to 250%, up to 255%, up to 260%, up to 265%, up to 270%, up to 275%, up to 280%, up to 285%, up to 290%, up to 295%, up to 300%, up to 305%, up to 310%, up to 315%, up to 320%, up to 325%, up to 330%, up to 335%, up to 340%, up to 345%, up to 350%, up to 355%, up to 360%, up to 365%, up to 370%, up to 375%, up to 380%, up to 385%, up to 390%, up to 395%, or up to 400%, preferably up to 104%, relative to the expression of the polypeptide of interest in a parent host cell which does not comprises a reduced or eliminated flagella activity, when cultivated under identical conditions. Preferably, the parent host cell is otherwise isogenic to the host cell according to the first aspect.

In one embodiment, the polypeptide of interest is a xylanase, and the expression of the xylanase is increased up to 4%, up to 5%, up to 10%, up to 15%, up to 20%, up to 25%, up to 30%, up to 35%, up to 40%, up to 45%, up to 50%, up to 55%, up to 60%, up to 65%, up to 69%, up to 70%, up to 75%, up to 80%, up to 85%, up to 90%, up to 100%, up to 105%, up to 110%, up to 115%, up to 120%, up to 125%, up to 130%, up to 135%, up to 140%, up to 145%, up to 150%, up to 155%, up to 160%, up to 165%, up to 170%, up to 175%, up to 180%, up to 185%, up to 190%, up to 195%, up to 200%, up to 205%, up to 210%, up to 215%, up to 220%, up to 225%, up to 230%, up to 235%, up to 240%, up to 245%, up to 250%, up to 255%, up to 260%, up to 265%, up to 270%, up to 275%, up to 280%, up to 285%, up to 290%, up to 295%, up to 300%, up to 305%, up to 310%, up to 315%, up to 320%, up to 325%, up to 330%, up to 335%, up to 340%, up to 345%, up to 350%, up to 355%, up to 360%, up to 365%, up to 370%, up to 375%, up to 380%, up to 385%, up to 390%, up to 395%, or up to 400%, preferably up to 69%, relative to the expression of the polypeptide of interest in a parent host cell which does not comprises a reduced or eliminated flagella activity, when cultivated under identical conditions. Preferably, the parent host cell is otherwise isogenic to the host cell according to the first aspect.

In one embodiment, the polypeptide of interest is a xanthan lyase, and the expression of the xanthan is increased up to 32%, up to 45%, up to 50%, up to 55%, up to 60%, up to 65%, up to 70%, up to 75%, up to 80%, up to 85%, up to 90%, up to 100%, up to 105%, up to 110%, up to 115%, up to 120%, up to 125%, up to 130%, up to 135%, up to 140%, up to 145%, up to 150%, up to 155%, up to 160%, up to 165%, up to 170%, up to 175%, up to 180%, up to 185%, up to 190%, up to 195%, up to 200%, up to 205%, up to 210%, up to 215%, up to 220%, up to 225%, up to 230%, up to 235%, up to 240%, up to 245%, up to 250%, up to 255%, up to 260%, up to 265%, up to 270%, up to 275%, up to 280%, up to 285%, up to 290%, up to 295%, up to 300%, up to 305%, up to 310%, up to 315%, up to 320%, up to 325%, up to 330%, up to 335%, up to 340%, up to 345%, up to 350%, up to 355%, up to 360%, up to 365%, up to 370%, up to 375%, up to 380%, up to 385%, up to 390%, up to 395%, or up to 400%, preferably up to 32%, relative to the expression of the polypeptide of interest in a parent host cell which does not comprises a reduced or eliminated flagella activity, when cultivated under identical conditions. Preferably, the parent host cell is otherwise isogenic to the host cell according to the first aspect.

The host cell may be any microbial cell useful in the recombinant production of one or more polypeptide of the present invention, e.g., a prokaryotic cell.

The introduction of DNA into a Bacillus cell may be affected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). The introduction of DNA into an E. coli cell may be affected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be affected by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell may be affected by electroporation (see, e.g., Choi et al., 2006, J. Microbiol. Methods 64: 391-397) or conjugation (see, e.g., Pinedo and Smets, 2005, Appl. Environ. Microbiol. 71: 51-57). The introduction of DNA into a Streptococcus cell may be affected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804), or conjugation (see, e.g., Clewell, 1981, Microbiol. Rev. 45: 409-436). However, any method known in the art for introducing DNA into a host cell can be used.

In one embodiment, the host cell further comprises a heterologous promoter operably linked to a second heterologous polynucleotide encoding a non-coding RNA molecule, said non-coding RNA molecule being configured to form a RNA:DNA heteroduplex with a target polynucleotide sequence of a flagellum gene by hybridizing or binding with/to said target polynucleotide sequence of the flagellum gene, or being configured to form a RNA:RNA heteroduplex with a target polynucleotide sequence of a RNA transcript of the flagellum gene by hybridizing or binding with/to said target polynucleotide sequence of the RNA transcript.

In a preferred embodiment, the second heterologous polynucleotide encodes a non-coding RNA molecule comprising, essentially consisting of or consisting of a polynucleotide sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the polynucleotide sequence of SEQ ID NO: 1, SEQ ID NO: 2, SEQ ID NO: 3, SEQ ID NO: 4, SEQ ID NO: 5 or SEQ ID NO: 6.

In another preferred embodiment, the second heterologous polynucleotide consists of, essentially consists of, or comprises a single guide RNA.

In yet another embodiment, the host cell comprises a third heterologous polynucleotide encoding a catalytically inactive site-directed polypeptide (CISP), the CISP being selected from the group consisting of a catalytically inactive CRISPR-associated protein, a catalytically inactive zinc finger nuclease (ZFN), a zinc finger, a catalytically inactive transcription activator-like effector nuclease (TALEN), a TALE, a catalytically inactive meganuclease, and a catalytically inactive MAD7/Cas12a endonuclease.

In a preferred embodiment, the CISP is configured to bind to a binding sequence of:
i) the polynucleotide sequence of the non-coding RNA molecule or ncRNA, OR
ii) the DNA or RNA polynucleotide sequence of the RNA:DNA heteroduplex, OR
iii) a RNA polynucleotide sequence of the RNA:RNA heteroduplex, such as the ncRNA of the RNA:RNA heteroduplex;

In a preferred embodiment, the CISP is configured to bind to the binding sequence of the polynucleotide sequence of the non-coding RNA molecule, such as a ncRNA molecule.

In another embodiment, the CISP is configured to bind to a guide RNA molecule.

In another embodiment the CISP and ncRNA or guide RNA are configured to increase the expression of a motI gene encoding a MotI polypeptide having at least 60% sequence identity to SEQ ID NO: 71.

In another aspect, the invention relates to a mutated bacterial host cell comprising a heterologous promoter operably linked to a first heterologous polynucleotide encoding one or more polypeptide of interest, wherein at least one flagellum gene is altered, said altered flagellum gene thereby encoding an altered flagellum polypeptide. Preferably the altered flagellum polypeptide affects the flagella activity of said host cell by reducing or eliminating the flagella activity of the host cell. In one embodiment the expression of the altered flagellum polypeptide is similar to the expression of the flagellum polypeptide in the non-mutated parent cell. In another embodiment the altered flagellum gene comprises an alteration selected from a nucleotide deletion, nucleotide insertion and a premature stop codon. Preferably, the alteration affects the functionality and/or activity of the flagellum, wherein the flagella activity is reduced or eliminated.

In another aspect the invention relates to a method of producing a mutant of a parent cell, comprising inactivating a polynucleotide encoding at least one flagellum polypeptide, which results in the mutant producing less of the flagellum polypeptide than the parent cell.

In another aspect the invention relates to a mutant cell produced by said method of producing a mutant of a parent cell.

In one embodiment the said mutant cell further comprises a gene encoding one or more native or heterologous protein.

In a further aspect the invention relates to a double-stranded inhibitory RNA (dsRNA) molecule comprising a subsequence of a polynucleotide sequence of at least one flagellum gene, wherein optionally the dsRNA is an siRNA or a miRNA molecule.

In one embodiment, the double-stranded inhibitory RNA (dsRNA) molecule is about 15, 16, 17, 18, 19, 20, 21, 22, 23, 24, 25 or more duplex nucleotides in length.

In a further aspect the invention relates to a method of inhibiting the expression of at least one flagellum polypeptide in a cell, comprising administering to the cell or expressing in the cell said double-stranded inhibitory RNA (dsRNA) molecule.

In a further aspect the invention relates to a mutant cell produced by said method of inhibiting the expression of at least one flagellum polypeptide.

### Methods of Production

In a second aspect, the present invention also relates to a method for producing one or more polypeptide of interest, the method comprising: i) providing a bacterial host cell according to the first aspect, ii) cultivating said host cell under conditions conducive for expression of the one or more polypeptide of interest; and iii) optionally recovering the one or more polypeptide of interest.

In one aspect, the cell is a Bacillus cell. In another aspect, the cell is a Bacillus subtilis cell. In another aspect, the cell is a Bacillus licheniformis cell.

In one embodiment, the cell is cultivated in a batch fermentation mode.

In one embodiment, the cell is cultivated in a fed-batch fermentation mode.

In one embodiment the duration of cultivation is at least 24 hours, at least 48 hours, at least 72 hours, at least 96 hours, or at least 120 hours.

In one embodiment the duration of cultivation is at least 24 hours, preferably the cultivation is a batch mode.

In one embodiment the duration of cultivation is at least 120 hours, preferably the cultivation is a fed-batch mode.

The host cells are cultivated in a nutrient medium suitable for production of the polypeptide using methods known in the art. For example, the cells may be cultivated by shake flask cultivation, or small-scale or large-scale fermentation (including continuous, batch, fed-batch, or solid-state fermentations) in laboratory or industrial fermentors in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide may be detected using methods known in the art that are specific for the polypeptides. These detection methods include, but are not limited to, use of specific antibodies, formation of an enzyme product, or disappearance of an enzyme substrate. For example, an enzyme assay may be used to determine the activity of the polypeptide

The polypeptide may be recovered using methods known in the art. For example, the polypeptide may be recovered from the fermentation medium by conventional procedures including, but not limited to, collection, centrifugation, filtration, extraction, spray-drying, evaporation, or precipitation. In one aspect, a whole fermentation broth comprising the polypeptide is recovered.

The polypeptide may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure polypeptides.

### Flagella Polypeptides

In some embodiments, the present invention relates to flagellum polypeptides having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, but less than 100% to the mature polypeptide of SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, and SEQ ID NO: 63, which contribute to a reduced or eliminated flagella activity. In some embodiments, the present invention relates to a flagellum polypeptide having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide of SEQ ID NO: 71, which contributes to a reduced or eliminated flagella activity when being present in the host cell.

In one aspect, the polypeptides differ by up to 10 amino acids, e.g., 1, 2, 3, 4, 5, 6, 7, 8, 9, or 10, from the mature polypeptide of SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, and SEQ ID NO: 63.

The flagellum polypeptide preferably comprises, consists essentially of, or consists of a variant of the amino acid sequence of SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, and SEQ ID NO: 63, or the mature polypeptide of the variant; or is a fragment thereof contributing to a reduced or eliminated flagellum activity.

In some embodiments, the present invention relates to flagella polypeptides contributing to a reduced or eliminated activity encoded by polynucleotides that hybridize under medium stringency conditions, medium-high stringency conditions, high stringency conditions, or very high stringency conditions with the full-length complement of the mature polypeptide coding sequence encoding the polypeptides of SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, and SEQ ID NO: 63, or the cDNA thereof (Sambrook et al., 1989, Molecular Cloning, A Laboratory Manual, 2d edition, Cold Spring Harbor, New York).

The coding flagellum polynucleotide sequence or a subsequence thereof, as well as the mature polypeptides of SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, and SEQ ID NO: 63, or a fragment thereof, may be used to design nucleic acid probes to identify and clone DNA encoding flagella polypeptides from strains of different genera or species according to methods well known in the art. Such probes can be used for hybridization with the genomic DNA or cDNA of a cell of interest, following standard Southern blotting procedures, in order to identify and isolate the corresponding gene therein. Such probes can be considerably shorter than the entire sequence, but should be at least 15, e.g., at least 25, at least 35, or at least 70 nucleotides in length. Preferably, the nucleic acid probe is at least 100 nucleotides in length, e.g., at least 200 nucleotides, at least 300 nucleotides, at least 400 nucleotides, at least 500 nucleotides, at least 600 nucleotides, at least 700 nucleotides, at least 800 nucleotides, or at least 900 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA or cDNA library prepared from such other strains may be screened for DNA that hybridizes with the probes described above and encodes a flagella polypeptide. Genomic or other DNA from such other strains may be separated by agarose or polyacrylamide gel electrophoresis, or other separation techniques. DNA from the libraries or the separated DNA may be transferred to and immobilized on nitrocellulose or another suitable carrier material. In order to identify a clone or DNA that hybridizes with a flagellum gene or a subsequence thereof, the carrier material is used in a Southern blot.

For purposes of the present invention, hybridization indicates that the polynucleotides hybridize to a labeled nucleic acid probe corresponding to (i) SEQ ID NO: 15, SEQ ID NO: 16, or SEQ ID NO: 17; (ii) the mature polypeptide coding sequence of SEQ ID NO: 15, SEQ ID NO: 16, or SEQ ID NO: 17; (iii) the cDNA sequence thereof; (iv) the full-length complement thereof; or (v) a subsequence thereof, such as the subsequences of SEQ ID NO: 18, SEQ ID NO: 19, SEQ ID NO: 20, SEQ ID NO: 21, SEQ ID NO: 22 or SEQ ID NO: 23; under medium to very high stringency conditions. Molecules to which the nucleic acid probe hybridizes under these conditions can be detected using, for example, X-ray film or any other detection means known in the art.

In some embodiments, the present invention relates to flagella polypeptides encoded by polynucleotides having a sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to the mature polypeptide coding sequence of SEQ ID NO: 15, SEQ ID NO: 16, or SEQ ID NO: 17 or the cDNA sequence thereof.

The polynucleotide encoding the polypeptide preferably comprises, consists essentially of, or consists of the polynucleotide with SEQ ID NO: 15, SEQ ID NO: 16, or SEQ ID NO: 17.

In some embodiments, the present invention relates to a polypeptide derived from a mature polypeptide of SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, and/or SEQ ID NO: 63 by substitution, deletion or addition of one or several amino acids in the mature polypeptide of SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, and/or SEQ ID NO: 63. In some embodiments, the present invention relates to variants of the mature polypeptide of SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, and/or SEQ ID NO: 63comprising a substitution, deletion, and/or insertion at one or more (e.g., several) positions. In one aspect, the number of amino acid substitutions, deletions and/or insertions introduced into the mature polypeptide of SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, and/or SEQ ID NO: 63 is up to 10, e.g., 1, 2, 3, 4, 5, 6, 7, 8, 9, or 10. In an embodiment, the polypeptide has an N-terminal extension and/or C-terminal extension of 1-10 amino acids, e.g., 1, 2, 3, 4, 5, 6, 7, 8, 9, or 10 amino acids. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding module. Preferably the amino acid changes affect the flagella activity by reducing or eliminating the flagella activity.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant molecules are tested for flagella activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mot Biol. 224: 899-904; Wlodaver et al., 1992, FEBS Lett. 309: 59-64. The identity of essential amino acids can also be inferred from an alignment with a related polypeptide.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

In some embodiments, the gene encoding the at least one flagellum polypeptide comprises a premature stop codon. In one embodiment, the flagellum gene comprising a premature stop codon encodes a shortened flagellum polypeptide affecting the flagella activity by reducing or eliminating the flagella activity. Preferably, the gene encoding the flagellum polypeptide or its coding region, is deleted fully or partially from the host cell genome, e.g. by homologous recombination. In a preferred embodiment the flagellum gene deletion affects the host cell's flagella activity by reducing or eliminating the flagella activity.

### Sources of Flagella Genes and Flagella Polypeptides

A flagellum gene or a flagellum polypeptide of the present invention may be obtained from microorganisms of any genus. For purposes of the present invention, the term “obtained from” as used herein in connection with a given source shall mean that the polypeptide encoded by a polynucleotide is produced by the source or by a strain in which the polynucleotide from the source has been inserted.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen and Zellkulturen GmbH (DSMZ), Centraalbureau Voor Schimmelcultures (CBS), and Agricultural Research Service Patent Culture Collection, Northern Regional Research Center (NRRL).

The flagella genes and flagella polypeptides may be identified and obtained from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. A polynucleotide encoding the polypeptide may then be obtained by similarly screening a genomic DNA or cDNA library of another microorganism or mixed DNA sample. Once a polynucleotide encoding a polypeptide has been detected with the probe(s), the polynucleotide can be isolated or cloned by utilizing techniques that are known to those of ordinary skill in the art (see, e.g., Sambrook et al., 1989, supra).

### Polynucleotides

The present invention also relates to isolated polynucleotides encoding at least one mutated flagellum polypeptide of the present invention as described herein. Preferably the mutated flagellum polypeptide affects the flagella activity and/or cell motility of the host cell by reducing or eliminating the host cell's flagella activity and/or cell motility.

The techniques used to isolate or clone a polynucleotide are known in the art and include isolation from genomic DNA or cDNA, or a combination thereof. The cloning of the polynucleotides from genomic DNA can be affected, e.g., by using the polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligation activated transcription (LAT) and polynucleotide-based amplification (NASBA) may be used. The polynucleotides may be cloned from a strain of Bacillus, or a related organism and thus, for example, may be a species variant of the polypeptide encoding region of the polynucleotide.

Modification of a polynucleotide encoding a mutated flagellum polypeptide of the present invention may be necessary for synthesizing polypeptides substantially similar to the polypeptide. The term “substantially similar” to the polypeptide refers to non-naturally occurring forms of the polypeptide. These polypeptides may differ in some engineered way from the polypeptide isolated from its native source, e.g., variants that reduce or eliminate flagella activity. The variants may be constructed on the basis of the polynucleotide presented as the mature polypeptide coding sequence of SEQ ID NO: 15, SEQ ID NO: 16 or SEQ ID NO: 17 or the cDNA sequence thereof, e.g., a subsequence thereof, and/or by introduction of nucleotide substitutions that result in a change in the amino acid sequence of the polypeptide, or by introduction of nucleotide substitutions that may give rise to a different amino acid sequence. For a general description of nucleotide substitution, see, e.g., Ford et al., 1991, Protein Expression and Purification 2: 95-107.

### Nucleic Acid Constructs

The present invention also relates to nucleic acid constructs comprising a polynucleotide of the present invention, wherein the polynucleotide optionally is operably linked to one or more control sequences that direct the expression of the coding sequence in a suitable host cell under conditions compatible with the control sequences.

The polynucleotide may be manipulated in a variety of ways to provide for expression of the at least one flagellum polypeptide, polypeptide of interest, CISP or ncRNA. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide that is recognized by a host cell for expression of a polynucleotide encoding a polypeptide or ncRNA of the present invention. The promoter contains transcriptional control sequences that mediate the expression of the polypeptide or transcription of the ncRNA. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the polynucleotide of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xylB genes, Bacillus thuringiensis cryIIIA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

The control sequence may also be a transcription terminator, which is recognized by a host cell to terminate transcription. The terminator is operably linked to the 3′-terminus of the polynucleotide encoding the polypeptide or ncRNA. Any terminator that is functional in the host cell may be used in the present invention.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, J. Bacteriol. 177: 3465-3471).

The control sequence may also be a leader, a non-translated region of an mRNA that is important for translation by the host cell. The leader is operably linked to the 5′-terminus of the polynucleotide encoding the polypeptide or ncRNA. Any leader that is functional in the host cell may be used.

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

It may also be desirable to add regulatory sequences that regulate expression of the polypeptide or ncRNA relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. Other examples of regulatory sequences are those that allow for gene amplification.

In a third aspect, the invention relates to a nucleic acid construct comprising a polynucleotide encoding at least one mutated flagellum polypeptide comprising or consisting of a polypeptide sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100% to the polypeptide sequence of any one of SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, and SEQ ID NO: 63.

In one embodiment a promoter is operably linked to the polynucleotide encoding the at least one mutated flagellum polypeptide. In a preferred embodiment, the mutated flagellum polypeptide affects the flagella activity or cell motility of the host cell, wherein the host cell's flagella activity or cell motility is reduced or eliminated. The promoter can be any homologous or heterologous promoter. Preferably the promoter is heterologous. In one embodiment, the nucleic acid construct according to the third aspect is integrated into the host cell genome by homologous recombination, wherein the non-mutated flagellum gene is deleted.

In one aspect, the nucleic acid construct comprises a heterologous promoter operably linked to a polynucleotide encoding a non-coding RNA molecule, wherein the non-coding RNA molecule is configured to form a RNA:DNA heteroduplex with a target polynucleotide sequence of at least one bacterial flagellum gene by hybridizing with said target polynucleotide sequence of the bacterial flagellum gene, or configured to form a RNA:RNA heteroduplex with a target polynucleotide sequence of a RNA transcript of the flagellum gene by hybridizing with said target polynucleotide sequence of the RNA transcript.

In one embodiment, the non-coding RNA molecule comprises, essentially consists of, or consists of a polynucleotide sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the polynucleotide sequence of (i) the polynucleotide sequence of SEQ ID NO: 15, SEQ ID NO: 16, or SEQ ID NO: 17; (ii) the transcript of SEQ ID NO: 15, SEQ ID NO: 16, or SEQ ID NO: 17; or (iii) the complementary polynucleotide sequence of SEQ ID NO: 15, SEQ ID NO: 16, or SEQ ID NO: 17.

In one embodiment, the non-coding RNA molecule comprises, essentially consists of, or consists of a polynucleotide sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the polynucleotide sequence of (i) the polynucleotide sequence of the coding strand of at least one bacterial flagellum gene selected from flgA, flgB, flgC, flgD, flgE, flgF, flgG, flgH, flgI, flgJ, flgK, flgL, flgM, flgN, flhA, flhB, flhC, flhD, flhE, flhF, flhG, flhO, flhP, fliA, fliD, fliE, fliF, fliG, fliH, filI, fliJ, fliK, fliL, fliM, fliN, fliO, fliP, fliQ, fliR, fliS, fliT, fliY, fliZ, hag, motA, motB, ylxF, swrD, cheY, cheB, cheA, cheVV, cheC, cheD, sigD, and swrB; (ii) the transcript of at least one bacterial flagellum gene selected from flgA, flgB, flgC, flgD, flgE, flgF, flgG, flgH, flgI, flgJ, flgK, flgL, flgM, flgN, flhA, flhB, flhC, flhD, flhE, flhF, flhG, flhO, flhP, fliA, fliD, fliE, fliF, fliG, fliH, filI, filJ, fliK, fliL, fliM, fliN, fliO, fliP, fliQ, fliR, fliS, fliT, fliY, fliZ, hag, motA, motB, ylxF, swrD, cheY, cheB, cheA, cheVV, cheC, cheD, sigD, and swrB; or (iii) the complementary polynucleotide sequence to the polynucleotide sequence of the coding strand of at least one bacterial flagellum gene selected from flgA, flgB, flgC, flgD, flgE, flgF, flgG, flgH, flgI, flgJ, flgK, flgL, flgM, flgN, flhA, flhB, flhC, flhD, flhE, flhF, flhG, flhO, flhP, fliA, fliD, fliE, fliF, fliG, fliH, fliI, fliJ, fliK, fliL, fliM, fliN, fliO, fliP, fliQ, fliR, fliS, fliT, fliY, fliZ, hag, motA, motB, ylxF, swrD, cheY, cheB, cheA, cheVV, cheC, cheD, sigD, and swrB.

In one embodiment, the non-coding RNA molecule comprises, essentially consists of, or consists of a polynucleotide sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the polynucleotide sequence of SEQ ID NO: 1, SEQ ID NO: 2, SEQ ID NO: 3, SEQ ID NO: 4, SEQ ID NO: 5 or SEQ ID NO: 6.

### Expression Vectors

The present invention also relates to recombinant expression vectors comprising a polynucleotide of the present invention, and optionally a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide or ncRNA at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

In a fourth aspect, the invention relates to an expression vector comprising a nucleic acid construct according to the third aspect.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a mini-chromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used. In one embodiment, the vector is integrated into the polynucleotide sequence of at least one flagellum gene, wherein the expression of the at least one flagellum gene is reduced or eliminated. In another embodiment, the vector is subject to homologous recombination or non-homologous recombination with the polynucleotide sequence of at least one flagellum gene, wherein the at least one flagellum gene or its coding region is deleted.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin, or tetracycline resistance.

The selectable marker may be a dual selectable marker system as described in WO 2010/039889. In one aspect, the dual selectable marker is a hph-tk dual selectable marker system.

The vector preferably contains an element(s) that permits integration of the vector into the host cell's genome or autonomous replication of the vector in the cell independent of the genome.

For integration into the host cell genome, the vector may rely on the polynucleotide's sequence encoding the polypeptide or the ncRNA or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination. In a preferred embodiment, the vector or non-homologous recombination enables the deletion or partial deletion of a gene encoding a flagella polypeptide.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMß1 permitting replication in Bacillus.

More than one copy of a polynucleotide of the present invention may be inserted into a host cell to increase production of a polypeptide of interest. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Removal or Reduction of Flagella Activity

The present invention also relates to methods of producing a mutant of a parent cell, which comprises disrupting or deleting a polynucleotide of a polynucleotide sequence, or a portion of a polynucleotide sequence, said polynucleotide sequence encoding a flagellum polypeptide of the present invention, which results in the mutant cell producing less of the flagellum polypeptide than the parent cell when cultivated under the same conditions. Additionally or alternatively, the flagella activity can be inhibited by the overexpression of a MotI polypeptide, such as the polypeptide of SEQ ID NO: 71.

The mutant cell may be constructed by reducing or eliminating expression of the polynucleotide using methods well known in the art, for example, insertions, disruptions, replacements, or deletions. In some embodiments, the flagellum polynucleotide is inactivated. The polynucleotide to be modified or inactivated may be, for example, the coding region or a part thereof essential for flagella activity, or a regulatory element required for expression of the coding region. An example of such a regulatory or control sequence may be a promoter sequence or a functional part thereof, i.e., a part that is sufficient for affecting expression of the polynucleotide. Other control sequences for possible modification include, but are not limited to, a leader, polyadenylation sequence, propeptide sequence, signal peptide sequence, transcription terminator, and transcriptional activator.

Modification or inactivation of the flagellum polynucleotide may be performed by subjecting the parent cell to mutagenesis and selecting for mutant cells in which expression of the flagellum polynucleotide has been reduced or eliminated. The mutagenesis, which may be specific or random, may be performed, for example, by use of a suitable physical or chemical mutagenizing agent, by use of a suitable oligonucleotide, or by subjecting the DNA sequence to PCR generated mutagenesis. Furthermore, the mutagenesis may be performed by use of any combination of these mutagenizing agents.

Examples of a physical or chemical mutagenizing agent suitable for the present purpose include ultraviolet (UV) irradiation, hydroxylamine, N-methyl-N′-nitro-N-nitrosoguanidine (MNNG), O-methyl hydroxylamine, nitrous acid, ethyl methane sulphonate (EMS), sodium bisulphite, formic acid, and nucleotide analogues.

When such agents are used, the mutagenesis is typically performed by incubating the parent cell to be mutagenized in the presence of the mutagenizing agent of choice under suitable conditions, and screening and/or selecting for mutant cells exhibiting reduced or no expression of

Modification or inactivation of the polynucleotide may be accomplished by insertion, substitution, or deletion of one or more nucleotides in the gene or a regulatory element required for transcription or translation thereof. For example, nucleotides may be inserted or removed so as to result in the introduction of a stop codon, the removal of the start codon, or a change in the open reading frame. Such modification or inactivation may be accomplished by site-directed mutagenesis or PCR generated mutagenesis in accordance with methods known in the art. Although, in principle, the modification may be performed in vivo, i.e., directly on the cell expressing the polynucleotide to be modified, it is preferred that the modification be performed in vitro as exemplified below.

Convenient ways to eliminate or reduce expression of a polynucleotide based on techniques of gene silencing (as presented in examples 2-8, and example 11), gene deletion and replacement (as presented in examples 9-10, and example 12-13), or gene disruption. For example, in the gene disruption method, a nucleic acid sequence corresponding to the endogenous polynucleotide is mutagenized in vitro to produce a defective nucleic acid sequence that is then transformed into the parent cell to produce a defective gene. By homologous recombination, the defective nucleic acid sequence replaces the endogenous polynucleotide. It may be desirable that the defective polynucleotide also encodes a marker that may be used for selection of transformants in which the polynucleotide has been modified or destroyed. In an aspect, the polynucleotide is disrupted with a selectable marker such as those described herein.

The present invention also relates to methods of inhibiting the expression of at least one flagellum polypeptide in a cell, comprising administering to the cell or expressing in the cell a double-stranded RNA (dsRNA) molecule, wherein the dsRNA comprises a subsequence of a polynucleotide of the present invention. In a preferred aspect, the dsRNA is about 15, 16, 17, 18, 19, 20, 21, 22, 23, 24, 25 or more duplex nucleotides in length.

The dsRNA is preferably a small interfering RNA (siRNA) or a micro RNA (miRNA). In a preferred aspect, the dsRNA is small interfering RNA for inhibiting transcription. In another preferred aspect, the dsRNA is micro RNA for inhibiting translation.

The present invention also relates to such double-stranded RNA (dsRNA) molecules, comprising a portion of the mature polypeptide coding sequence of at least one bacterial flagellum gene selected from flgA, flgB, flgC, flgD, flgE, flgF, flgG, flgH, flgI, flgJ, flgK, flgL, flgM, flgN, flhA, flhB, flhC, flhD, flhE, flhF, flhG, flhO, flhP, fliA, fliD, fliE, fliF, fliG, fliH, fliI, fliJ, fliK, fliL, fliM, fliN, fliO, fliP, fliQ, fliR, fliS, fliT, fliY, fliZ, hag, motA, motB, ylxF, swrD, cheY, cheB, cheA, cheW, cheC, cheD, sigD, and swrB, preferably a portion of the sequence of SEQ ID NO: 15, SEQ ID NO: 16 or SEQ ID NO: 17, for inhibiting expression of at least one flagellum polypeptide in a cell. In one embodiment the dsRNA is directed to a polynucleotide sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to a polynucleotide sequence selected from the list of SEQ ID NO: 18 (flgE), SEQ ID NO: 19 (flgE), SEQ ID NO: 20 (fliR), SEQ ID NO: 21 (fliR), SEQ ID NO: 22 (flhG) and SEQ ID NO: 23 (flhG).

In one embodiment, the level of flagella target gene mRNA is decreased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, relative to the flagella target gene mRNA level in a parent host cell which does not comprises a reduced or eliminated flagella activity, when cultivated under identical conditions. Preferably, the parent host cell is otherwise isogenic to the host cell according to the first aspect.

While the present invention is not limited by any particular mechanism of action, the dsRNA can enter a cell and cause the degradation of a single-stranded RNA (ssRNA) of similar or identical sequences, including endogenous mRNAs. When a cell is exposed to dsRNA, mRNA from the homologous gene is selectively degraded by a process called RNA interference (RNAi).

The dsRNAs of the present invention can be used in gene-silencing. In one aspect, the invention provides methods to selectively degrade RNA using a dsRNAi of the present invention. The process may be practiced in vitro, ex vivo or in vivo. In one aspect, the dsRNA molecules can be used to generate a loss-of-function mutation in a cell, an organ or an animal. Methods for making and using dsRNA molecules to selectively degrade RNA are well known in the art; see, for example, U.S. Pat. Nos. 6,489,127; 6,506,559; 6,511,824; and 6,515,109.

The gene disruption can also be utilized by targeting the at least one flagellum gene with a site-specific endonuclease to generate out-of-frame insertions or deletions in the flagellum polynucleotide. Suitable site-specific endonucleases are well known and include ZFN, TALEN, meganucleases or RNA-guided endonucleases such as CRISPR/Cas9 or CRISPR/Cas12a, e.g. MAD7.

The present invention further relates to a mutant cell of a parent cell that comprises a disruption or deletion of a polynucleotide encoding at least one flagellum polypeptide or a control sequence thereof or a silenced gene encoding the polypeptide, which results in the mutant cell producing less of the flagellum polypeptide or no flagellum polypeptide compared to the parent cell.

The polypeptide-deficient mutant cells are particularly useful as host cells for expression of native and heterologous polypeptides. Therefore, the present invention further relates to methods of producing one or more native or heterologous polypeptide, comprising (a) cultivating the mutant cell under conditions conducive for production of the one or more polypeptide; and (b) recovering the polypeptide. The term “heterologous polypeptides” means polypeptides that are not native to the host cell, e.g., a variant of a native protein. The host cell may comprise more than one copy of a polynucleotide encoding the native or heterologous polypeptide.

The methods used for cultivation and purification of the product of interest may be performed by methods known in the art.

The present invention further relates to a mutant cell of a parent cell that comprises a disruption or deletion of a polynucleotide encoding the polypeptide or a control sequence thereof or a silenced gene encoding the at least one flagellum polypeptide, which results in the mutant cell producing less of the flagellum polypeptide or no polypeptide compared to the parent cell.

The flagellum polypeptide-deficient mutant cells are useful as host cells for expression of native and heterologous polypeptides. Therefore, the present invention further relates to methods of producing one or more native or heterologous polypeptide, comprising (a) cultivating the mutant cell under conditions conducive for production of the one or more polypeptide; and (b) recovering the polypeptide. The term “heterologous polypeptides” means polypeptides that are not native to the host cell, e.g., a variant of a native protein.

The methods used for cultivation and purification of the product of interest may be performed by methods known in the art.

### EXAMPLES

### Media

Bacillus strains were grown on LB agar (10 g/L Tryptone, 5 g/L yeast extract, 5 g/L NaCl, 15 g/L agar) plates or in TY liquid medium (20 g/L Tryptone, 5 g/L yeast extract, 7 mg/L FeCl₂, 1 mg/L MnCl₂, 15 mg/L MgCl₂). To select for erythromycin resistance, agar and liquid media were supplemented with 5 μg/ml erythromycin. To select for chloramphenicol resistance, agar and liquid media were supplemented with 6 μg/ml chloramphenicol. To select for spectinomycin resistance, agar and liquid media were supplemented with 120 μg/mL spectinomycin. Growth media for strains carrying the alr gene disruption was supplemented with D-alanine to a final concentration of 0.4 mg/mL. Transformation of Bacilli was in Spizizen I medium which consists of 1×Spizizen salts (6 g/L KH₂PO₄, 14 g/L K₂HPO₄, 2 g/L (NH₄)₂SO₄, 1 g/L sodium citrate, 0.2 g/L MgSO₄ pH 7.0), 0.5% glucose, 0.1% yeast extract and 0.02% casein hydrolysate.

### Molecular Biological Methods

DNA manipulations and transformations were performed by standard molecular biology methods as described in:
Sambrook et al. (1989): Molecular cloning: A laboratory manual. Cold Spring Harbor laboratory, Cold Spring Harbor, NY.
Ausubel et al. (eds) (1995): Current pro7tocols in Molecular Biology. John Wiley and Sons.
Harwood and Cutting (eds) (1990): Molecular Biological Methods for Bacillus. John Wiley and Sons.

Enzymes for DNA manipulation were obtained from New England Biolabs, Inc. and used essentially as recommended by the supplier.

Competent cells and transformation of B. subtilis was obtained as described in Yasbin et al. (1975, Transformation and transfection in lysogenic strains of Bacillus subtilis: evidence for selective induction of prophage in competent cells. J. Bacteriol. 121, 296-304). Genomic DNA was prepared by using the commercially available QIAamp DNA Blood Kit from Qiagen. The respective DNA fragments were amplified by PCR using the Phusion Hot Start DNA Polymerase system (Thermo Scientific). PCR amplification reaction mixtures contained 1 μL (0.1 μg) of template DNA, 1 μL of sense primer (20 pmol/μL), 1 μL of anti-sense primer (20 pmol/μL), 10 μL of 5× PCR buffer with 7.5 mM MgCl₂, 8 μL of dNTP mix (1.25 mM each), 39 μL water, and 0.5 μL (2 U/) DNA polymerase. A thermocycler was used to amplify the fragment. The PCR products were purified from a 1.2% agarose gel with 1× TBE buffer using the Qiagen QIAquick Gel Extraction Kit (Qiagen, Inc., Valencia, CA) according to the manufacturer's instructions.

The condition for SOE-PCR is as follows: purified PCR products were used in a subsequent PCR reaction to create a single fragment using splice overlapping PCR (SOE) using the Phusion Hot Start DNA Polymerase system (Thermo Scientific) as follows. The PCR amplification reaction mixture contained 50 ng of each of the three gel purified PCR products. Primers complementary to the very 3′-end of each strand of the outer PCR products were added and a thermocycler was used to assemble and amplify the SOE fragment.

The condition for POE-PCR is as follows: purified PCR products were used in a subsequent PCR reaction to create a single fragment using splice overlapping PCR (SOE) using the Phusion Hot Start DNA Polymerase system (Thermo Scientific) as follows. The very 5′ end fragment and the very 3′ end fragment have complementary end which will allow the SOE to concatemer into the POE PCR product. The PCR amplification reaction mixture contained 50 ng of each of the three gel purified PCR products. POE PCR was performed as described in (You, C et al (2017) Methods Mol. Biol. 116, 183-92).

### Swimming/Motility Assay

Strains were inoculated into 10 mL TY ON at 37 C 250 rpm. The ON cultures were dilute to OD(450 nm)=0.05 in 10 mL and grown for 4 hrs at 37 C 250 rpm. 2 μL culture was spotted onto a freshly made 0.26% LB-agar plate. Casting of the plate was done as follows: LB-agar was melted in a microwave oven. 15 mL LB-agar was poured into a petri-dish in a LAF-bench. Dried for 10 min. From the 10 mL ON culture, 2 μL culture was spotted onto the plate. Dried for additional 5 min. Placed at 37 C in incubator ON.

### Biolector Fermentations

Strains were fermented in flower plates (MTP-48-B) in 1 mL TY media for 24 hr at 37 C, 1000 rpm in the Biolector (m2p-labs). The cultivation plates were inoculated from an over-night culture grown in a M-tube in 10 mL TY media at 37 C, 250 rpm. The flower plates were inoculated to a OD(450 nm) of 0.05.

### Amylase Assay

Amylase activity was measured in culture supernatants using the AMYL (Roche/Hitachi #11876473 001). Culture supernatants from DWP were diluted to 1/50 in Stabilizer buffer (0.03M CaCl2; 0.0083% Brij 35). Samples from bioreactor samples were diluted in Stabilizer buffer. Reagent 1 and reagent 2 of the AMYL kit was mixed 10:1 to generate the assay substrate. 20 μL diluted sample was mixed with 180 μL assay substrate. Assay was incubated as 37 C w/shaking for 30 min. Absorbance was measured at 40 5nm in plate reader. An amylase standard was included from the final activity value, KNU(N)/g, was determined.

### Protease Assay

The serine endopeptidase hydrolyses the substrate N-Succinyl-Ala-Ala-Pro-Phe p-nitroanilide. The reaction was performed at Room Temperature at pH 9.0. The release of pNA results in an increase of absorbance at 405 nm and this increase is proportional to the enzymatic activity measured against a standard.

### Xanthan Lyase Assay

Reducing ends assay using Xanthan (Keltrol T) as a substrate, at 50° C. pH 7.0. The reaction is stopped by an alkaline reagent containing PAHBAH and bismuth that forms complexes with reducing sugar. The complex formation results in colour production which can be read at 405 nm by a spectrophotometer. The produced colour is proportional to the Xanthan Lyase activity.

### Xylanase Assay:

Reducing ends assay using Wheat Arabinoxylan (WAXY-M, Megazyme) as a substrate, at 50° C. pH 6.0. The reaction is stopped by an alkaline reagent containing PAHBAH and bismuth that forms complexes with reducing sugar. The complex formation results in colour pro-duction which can be read at 405 nm by a spectrophotometer. The produced colour is proportional to the Xylanase activity.

### RNA Extraction

Samples for qRT-PCR were obtained from overnight cultures in YT medium of each strain in triplicates diluted to OD₄₅₀ 0.05 before harvesting at OD₄₅₀ ˜0.8. For all samples, cells were collected at 3,220 g for 4 minutes at 4° C. Pellets were vortexed in 0.5 ml glass beads (Sigma #G8772), 1 ml extraction buffer (10 mM NaOAc, 150 mM sucrose, 1% SDS), and 1 ml phenol:chloroform 5:1 pH 4.5 (ThermoFisher #AM9720) for 4 minutes and glass beads were removed. Samples were incubated for 5 minutes at 65 ° C. before freezing in liquid nitrogen and centrifuged at 13,000 g for 20 minutes at 4° C. before transferring the aqueous phase to repeat the hot phenol extraction. The aqueous phase was then transferred to 1 volume of chloroform and inverted before centrifugation at 13,000 g for 10 minutes at 4° C. for phase separation. RNA was finally precipitated in 1 volume isopropanol at room temperature for 10 minutes before centrifugation at 15,000 g for 45 minutes at 4° C. RNA pellets were washed with 70% ethanol and dissolved in water. DNase digestion was performed for qRT-PCR samples using TURBO DNase (Invitrogen #AM2238) and purified using RNA Clean & Concentrator (Zymo research #R1016) according to manufacturer's instructions. DNase digestion was performed for fermentation RNA-seq samples using DNase I (Qiagen #79254) and purified using RNeasy MinElute Cleanup Kit (Qiagen #74204) according to manufacturer's instructions. RNA integrity was assessed using gel electrophoresis or bioanalyzer.

### qRT-PCR

Quantitative RT-PCR was performed using Brilliant III Ultra-Fast SYBR Green qRT-PCR Master Mix (Agilent Technologies #600886) according to manufacturer's protocol with 5 ng RNA in 10 μl reactions using 0.5 μM of each oligo (oligos listed in SEQ ID NO: 80-91). Each of three biological replicates were quantified in technical duplicates using Quantstudio 6 Flex (Applied Biosystems #4485694) incubating at 50° C. for 10 minutes, 95 ° C. for 3 minutes and 40 cycles of 95° C. for 5 seconds and 60° C. for 15 seconds. Fold changes were calculated using the 2^{-ΔΔCt} method and citA was used as reference gene.

### Plasmids

pE194 (SEQ ID NO: 9): Plasmid isolated from Staphylococcus aureus (Horinouchi, S 4 Weisbium, B. Journal of Bacteriology, 1982, 150(2):804-814).
pTK0001: Plasmid for expressing ncRNAs in B. subtilis (present disclosure)

### Strains

Bacillus subtilis 168: Kunst F, Ogasawara N, Moszer I, et al., Nature. 1997 Nov. 20; 390(6657). 249-56.
AEB2718: Bacillus subtilis 168 with deletions in the genes sigF, nprE, aprE, amyE, and srfAC, rendering them all inactive. Same deletions as described in Sloma, A., and L. Christianson (1999). Nucleic acids encoding a polypeptide having protease activity. U.S. Pat. No. 5,891,701.
ThKK0007: B. subtilis AEB2718, pel::P4199-JE1zyn-cat
ThKK0016: B. subtilis ThKK0007; amyE::spec P4199′-CISP
ThKK0086: B. subtilis ThKK0016; amyE::spec P4199′-CISP, ′dal−
ThKK0108: B. subtilis ThKK0086; amyE::spec P4199′-CISP, ′dal+Pq_ncRNA::GFP
ThKK0273: B. subtilis ThKK0086; amyE::spec P4199′-CISP, ′dal+Pq_ncRNA::ncRNA_19_0003_1 35
ThKK0274: B. subtilis ThKK0086; amyE::spec P4199′-CISP, ′dal+Pq_ncRNA::ncRNA_19_0003_2
ThKK0275: B. subtilis ThKK0086; amyE::spec P4199′-CISP, ′dal+Pq_ncRNA::ncRNA_19_0003_3
ThKK0276: B. subtilis ThKK0086; amyE::spec P4199′-CISP, ′dal+Pq_ncRNA::ncRNA_19_0003_4
ThKK0277: B. subtilis ThKK0086; amyE::spec P4199′-CISP, ′dal+Pq_ncRNA::ncRNA_19_0003_5
ThKK0278: B. subtilis ThKK0086; amyE::spec P4199′-CISP, ′dal+Pq_ncRNA::ncRNA_19_0003_6
BT11018: B. subtilis ThKK0007, flgE::ERM
BT11019: B. subtilis AEB2718, flgE::ERM
BT11103: B. subtilis AEB2718, amyE::Pcons-aprH-cat
BT11104: B. subtilis BT11019, amyE::Pcons-aprH-cat, flgE::ERM
BT11105: B. subtilis AEB2718, amyE::Pcons-Nattokinase-cat
BT11106: B. subtilis BT11019, amyE::Pcons-Nattokinase-cat, flgE::ERM
BT11109: B. subtilis AEB2718, amyE::Pcons-Xylanase-cat
BT11110: B. subtilis BT11019, amyE::Pcons-Xylanase-cat, flgE::ERM
BT11111: B. subtilis AEB2718, pel::P4199-Xathan lyase-cat
BT11112: B. subtilis BT11019, pel::P4199-Xathan lyase, flgE::ERM

### Primers

TABLE 1
Primer and sequence overview

| SEQ ID NO: | Name of molecule |
|---|---|
| 1 | flgE ncRNA_19_003_1 |
| 2 | flgE ncRNA_19_003_2 |
| 3 | fliR ncRNA_19_003_3 |
| 4 | fliR ncRNA_19_003_4 |
| 5 | flhG ncRNA_19_003_5 |
| 6 | flhG ncRNA_19_003_6 |
| 7 | GFP ncRNA |
| 8 | JE1 amylase polypeptide |
| 9 | PE194 |
| 10 | JE1zyn expression cassette |
| 11 | Upstream flanking region for alr integration |
| 12 | Downstream flanking region for alr integration |
| 13 | Up_frag sequence for HTP integration into alr |
| 14 | Down_frag sequence for HTP integration into alr |
| 15 | flgE gene sequence |
| 16 | fliR gene sequence |
| 17 | flhG gene sequence |
| 18 | flgE target sequence_1 |
| 19 | flgE target sequence_2 |
| 20 | fliR target sequence_3 |
| 21 | fliR target sequence_4 |
| 22 | flhG target sequence_5 |
| 23 | flhG target sequence_6 |
| 24 | FlgB polypeptide |
| 25 | FlgC polypeptide |
| 26 | FlgD polypeptide |
| 27 | FlgE polypeptide |
| 28 | FlgK polypeptide |
| 29 | FlgL polypeptide |
| 30 | FlhA polypeptide |
| 31 | FlhB polypeptide |
| 32 | FlhF polypeptide |
| 33 | FlhG polypeptide |
| 34 | FlhO polypeptide |
| 35 | FlhP polypeptide |
| 36 | FliD polypeptide |
| 37 | FliE polypeptide |
| 38 | FliF polypeptide |
| 39 | FliG polypeptide |
| 40 | FliH polypeptide |
| 41 | FliI polypeptide |
| 42 | FliJ polypeptide |
| 43 | Flik polypeptide |
| 44 | FliL polypeptide |
| 45 | FliM polypeptide |
| 46 | FliP polypeptide |
| 47 | FliQ polypeptide |
| 48 | FliR polypeptide |
| 49 | FliY polypeptide |
| 50 | FliZ (FliO) polypeptide |
| 51 | Hag polypeptide |
| 52 | MotA polypeptide |
| 53 | MotB polypeptide |
| 54 | YIxF polypeptide |
| 55 | SwrD polypeptide |
| 56 | CheY polypeptide |
| 57 | CheB polypeptide |
| 58 | CheA polypeptide |
| 59 | CheW polypeptide |
| 60 | CheC polypeptide |
| 61 | CheD polypeptide |
| 62 | SigD polypeptide |
| 63 | SwrD polypeptide |
| 64 | oTK0558 |
| 65 | oTK0578 |
| 66 | oTK0579 |
| 67 | oTK0561 |
| 68 | oTK0577 |
| 69 | oTK0580 |
| 70 | flgE deletion fragment |
| 71 | Motl polypeptide |
| 72 | protease polypeptide |
| 73 | nattokinase polypeptide |
| 74 | xylanase polypeptide |
| 75 | xanthan lyase polypeptide |
| 76 | Pcons-aprH-cat (protease cassette) |
| 77 | Pcons-nattokinase-cat (nattokinase cassette) |
| 78 | Pcons-xylanase-cat (xylanase cassette) |
| 79 | P4199-Xanthan lyase-cat (xanthan lyase cassette) |
| 80 | citA-fwd oligo |
| 81 | citA-rev oligo |
| 82 | flgE-fwd oligo |
| 83 | flgE-rev oligo |
| 84 | flgB-fwd oligo |
| 85 | flgB-rev oligo |
| 86 | fliR-fwd oligo |
| 87 | fliR-rev oligo |
| 88 | flhG-fwd oligo |
| 89 | flhG-rev oligo |
| 90 | cheD-fwd oligo |
| 91 | cheD-rev oligo |

### ncRNA Target Sequences

TABLE 2
List of ncRNA target sequences and target genes in B. subtilis

| | | | | ncRNA | |
|---|---|---|---|---|---|
| Target | Gene | | | SEQ | |
| gene | ID | Reference | ncRNA name | ID NO: | Strain |
| flgE | 936497 | NC_000964.3 | ncRNA_19_003_1 | 1 | ThKK0273 |
| flgE | 936497 | NC_000964.3 | ncRNA_19_003_2 | 2 | ThKK0274 |
| fliR | 940130 | NC_000964.3 | ncRNA_19_003_3 | 3 | ThKK0275 |
| fliR | 940130 | NC_000964.3 | ncRNA_19_003_4 | 4 | ThKK0276 |
| flhG | 940123 | NC_000964.3 | ncRNA_19_003_5 | 5 | ThKK0277 |
| flhG | 940123 | NC_000964.3 | ncRNA_19_003_6 | 6 | ThKK0278 |
| GFP | — | — | GFP ncRNA | 7 | ThKK0108 |

### Example 1: Generation of Amylase Strain

Synthetic DNA was ordered containing an expression cassette for JE1zyn amylase (SEQ ID NO: 10) under control of the P4199 promoter. The JE1zyn amylase is a codon optimized version of the JE1 amylase from Bacillus halmapalus. The P4199 promoter was earlier described in WO1993010249. The construct was inserted into the pel locus in AEB2718 resulting in strain Th KK0007.

### Example 2: Generation of Amylase Strain With CISP

An expression cassette was inserted at the amyE locus with the cisp gene encoding the CISP which is expressed from the P4199 promoter, into ThKK0007. The P4199 promoter was earlier described in WO1993010249. The resulting strain was named ThKK0016

### Example 3: Generation of ncRNA::GFP expression plasmid

To facilitate the binding of the CISP to a target DNA sequence of a flagellum gene, the presence of a ncRNA was necessary, said ncRNA being configured to bind to the DNA sequence of the flagellum target gene and enabling the binding of the CISP to the RNA:DNA heteroduplex in order to reduce or eliminate the expression of the flagellum target gene. The ncRNA was expressed from the amyQ promoter from Bacillus amyloliquefaciens (WO1999043835A2). The expression cassettes, including the Pq promoter, ncRNA target sequence, ncRNA constant domain, and terminator, were ordered from GeneArt as a DNA string with a ncRNA target sequence directed towards GFP. The ncRNA expression cassette was cloned into pE194 by POE-PCR. POE-PCR was performed as described in (You, C et al (2017) Methods Mol. Biol. 116, 183-92). The resulting plasmid is pTK0001.

Transformants were plated on erythromycin (1 μg/mL) LBPG plates. Growing colonies were restricken on erythromycin (1 μg/mL) LBPG plates and plasmid was purified and sequenced. Correct plasmid was saved as pTK0001

### Example 4: Generation of Recipient Strain for Chromosomal Integration of ncRNA

The alr locus was inactivated, rendering the strain unable to grow without D-alanine supplementation to the growth medium, by inserting by the neomycin resistance gene NeoR according to standard laboratory practice. The alr disruption was done in the ThKK0016 resulting in the ThKK0086 strain.

### Example 5: Generation of Strain With Chromosomally Integrated ncRNA Against GFP (ThKK0108)

To integrate the ncRNA expression construct, the episomal cloned ncRNA::GFP expression cassette was moved from pTK0001 into the alr locus of an appropriate acceptor strain (ThKK0086). The flanking sequences, which directs the homologues recombination, were amplified from a wild type strain carrying the functional alr (in this case ThKK0007). The upstream flanking region sequence is listed as SEQ ID NO: 11 and the downstream flanking region is listed as SEQ ID NO: 12. The flanking regions and ncRNA::GFP expression cassette was assembled by SOE PCR.

The transformation of the SOE PCR product, was performed as previously described in Yasbin et al. (1975, Transformation and transfection in lysogenic strains of Bacillus subtilis: evidence for selective induction of prophage in competent cells. J. Bacteriol. 121, 296-304), with the additional of 400 μL 10 mg/mL D-alanine to 10 mL Spitz transformation media. The resulting strain with the ncRNA::GFP is ThKK0108

### Example 6: Generation of Strains With Chromosomally Integrated ncRNA Based on ThKK0108

For the high-throughput cloning of ncRNA, the 20 bp ncRNA target sequence in the chromosomal integrated ncRNA::GFP (ThKK0108), was substituted with new 20 bp target sequences by oligo overlap.

The new ncRNA target sequence oligos were ordered in plates from Eurofins Genomics. The template for the upstream integration fragment (Up_frag) is made on genomic DNA from ThKK0108 (see SEQ ID NO: 13 for Up_frag sequence), and the template for the downstream integration fragment (Down_frag) is made on genomic DNA from ThKK0108 (see SEQ ID NO: 14 for Down_frag sequence). The ncRNA_up oligo was used to generate the up_ncRNA_fragment and the ncRNA_down oligo was used to generate the down_ncRNA _fragment (Bp, SEQ ID NO: 12). These PCRs generated the up- and downstream flanking regions for integration into alr Template Ap and Bp were then combined and the new SOE fragment carrying the new ncRNA sequence was created (see Table 2 for details). The SOE PCR is performed using a GC buffer (Phusion Hot Start DNA-Polymerase system (Thermo Scientific) with added 2% DMSO). The SOE was transformed in ThKK0086 as described above.

### Example 7: Effects of Directing CISP to Flagellum Operon

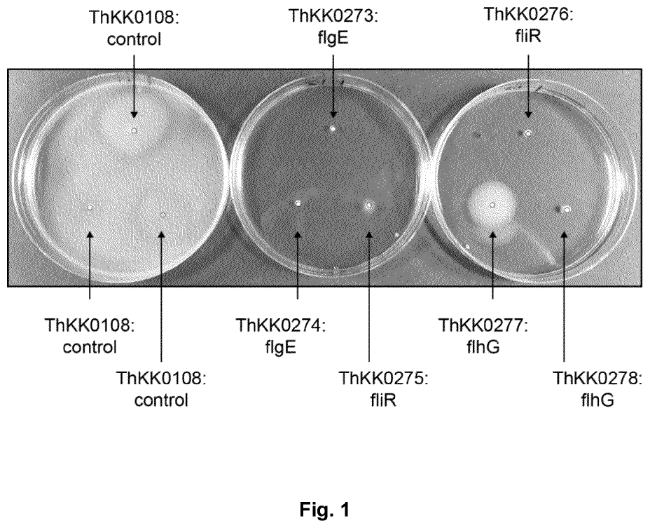

To evaluate the effect of directing the CISP machinery to the flagellum operon on the cells ability to form funtional flagella, ncRNAs were cloned to target three flagella genes in the flagellum operon (see Table 2).The DNA sequence of flgE, fliR, and flhG are found as SEQ ID NO: 15 (flgE), SEQ ID NO: 16 (fliR), and SEQ ID NO: 17 (flhG). The target nucleotide sequences of the targeted flagella genes are found as SEQ ID NO: 18 (flgE), SEQ ID NO: 19 (flgE), SEQ ID NO: 20 (fliR), SEQ ID NO: 21 (fliR), SEQ ID NO: 22 (flhG) and SEQ ID NO: 23 (flhG). A swimming/motility assay was performed to evaluate the effect of the block of the flagellum operon (FIG. 1: Swimming assay, ncRNAs directed to Flagellum operon inhibits swimming, black circle indicates where the strains were deposited on the plate). The control strain (ThKK0108) spreads out and covers the full plate, displaying its capability to swim in the thin agar plate (FIG. 1, left plate). All six strains carrying ncRNA directed against the flagellum operon (ThKK0273: flgE, ThKK0274: flgE, ThKK0275: fliR, ThKK0276: fliR, ThKK0277: flhG, and ThKK0278: flhG) resulted in a decreased swimming capacity compared to the control ncRNA::GFP (ThKK0108), indicating that the flagella functionallity is compromissed (FIG. 1, middle and right plate) by disrupting the gene expression of any one of the flagella genes flgE, fliR or flhG.

### Example 8: Increased Amylase Activity After Disruption of the Flagellum Operon

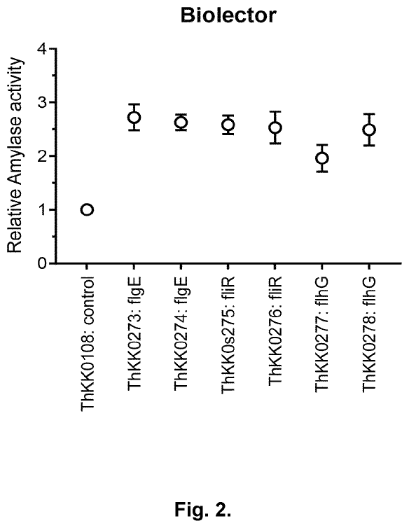

ThKK0273 (disrupted flgE expression), ThKK0274 (disrupted flgE expression), ThKK0275 (disrupted fliR expression), ThKK0276 (disrupted fliR expression), ThKK0277 (disrupted flhG expression), and ThKK0278 (disrupted flhG expression) were cultivated in the Biolector setup to evaluate the effect of the flagellum disruption on amylase activity and yield. ThKK0108 (intact flagellum gene expression) was included as control and the results of the activity assay can be seen in FIG. 2 (FIG. 2: JE1 amylase activity data from Biolector fermentations, normalized to ncRNA::GFP, n=3, error bars depict standard deviation).

As shown in FIG. 2, in the biolector ThKK0273 displayed 2,76 fold (±0.25, CV(%)=9.0) increase in amylase activity compared to ThKK0108, wherease ThKK0274 displayed 2.67 fold (±0.10, CV(%)=3.8) increase in amylase activity compared to ThKK0108, indicating that the disruption of flgE gene expression increases the activity and/or yield of recombinant protein.

As further shown in FIG. 2, ThKK0275 displayed 2.63 fold (±0.13, CV(%)=5.1) increase in amylase activity compared to ThKK0108, whereas ThKK0276 displayed 2.61 fold (±0.23, CV(%)=8.7) increase in amylase activity compared to ThKK0108, indicating that the disruption of fliR gene expression increases the activity and/or yield of recombinant protein.

As further shown in FIG. 2, ThKK0277 displayed 2.02 fold (±0.20, CV(%)=10.6) increase in amylase activity compared to ThKK0108, and ThKK0278 displayed 2.56 fold (±0.26, CV(%)=10.1) increase in amylase activity compared to ThKK0108, indicating that the disruption of flhG gene expression increases the activity and/or yield of recombinant protein.

### Example 9: Deletion of flgE in ThKK0007

The flgE gene encoding the flagellum hook protein was deleted in the ThKK0007 strain by substituting the open reading frame of flgE with the erythromycin (Erm) selection marker followed by a hairpin terminator. The individual fragments for the deletion are identified as SEQ ID NOs 64 to 69. The fragments are assembled by SOE PCR according to standard laboratory procedure. See SEQ ID NO: 70 for full deletion fragment. The SOE was transformed into ThKK0007. Transformants were plated onto erythromycin (1 μg/mL) LBPG plates. Colonies were plated on erythromycin (1 μg/mL) LBPG plates. Growing colonies were restricken on erythromycin (1 μg/mL) LBPG plates and sequenced. The resulting deletion strain was named BT11018. The deletion was also made in AEB2718, resulting in BT11019.

TABLE 3
PCR scheme for SOE generation for flgE deletion

| 1. PCRs | | | | | | |
|---|---|---|---|---|---|---|
| | Template DNA | | | SOE-PCR | | |
| Fragment | from | Primers | Size | Template | Primer | Size |
| Upstream flgE | Genomic DNA | oTK0558 (SEQ | 1.5 kb | Upstream | oTK0558 | 4.0 kb |
| | from | ID NO: 64) + | | flgE + | (SEQ ID | |
| | ThKK0007 | oTK0578 (SEQ | | Downstream | NO: 64 + | |
| | | ID NO: 65) | | flgE + Erm | oTK0561 | |
| Downstream flgE | Genomic DNA | oTK0579 (SEQ | 1.5 kb | | (SEQ ID | |
| | from | ID NO: 66) + | | | NO: 67) | |
| | ThKK0007 | oTK0561 (SEQ | | | | |
| | | ID NO: 67) | | | | |
| Erm marker | pE194 (SEQ | oTK0577 (SEQ | 1.0 kb | | | |
| | ID NO: 9) | ID NO: 68) + | | | | |
| | | oTK0580 (SEQ | | | | |
| | | ID NO: 69) | | | | |

### Example 10: Increased Amylase Activity Observed After Deletion of the flgE Gene

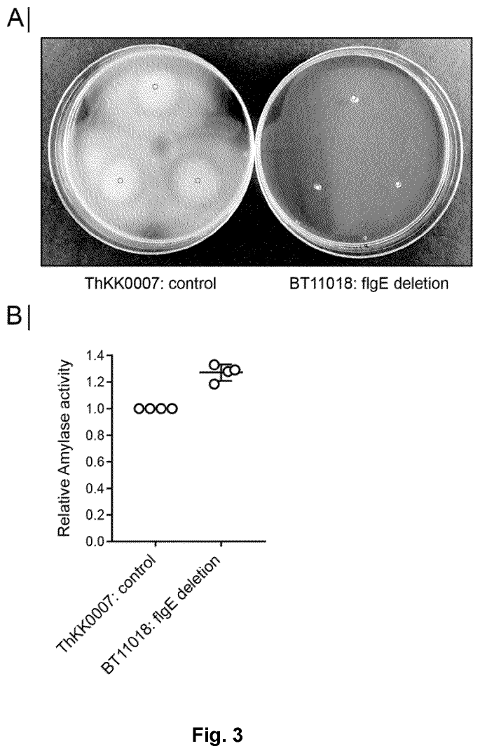

To evaluate the performance of BT11018, the strain was fermented in a Biolector and subjected to a swimming assay. ThKK0007 was included as control. BT11018 displayed no swimming compared to the control ThKK0007 (FIG. 3A) indicating that the reduced or eliminated expression of the flgE gene decreases or eliminates flagella activity. As shown in FIG. 3B (n=4, error bars depict standard deviation) BT11018 displayed 1,27 fold (±0.06, CV(%)=4.8) increase in amylase activity compared to ThKK0007, clearly indicating that the disruption of flgE gene expression increases the activity and/or yield of recombinant protein. Flagellum gene deletion has therefore been found to give similar effects as flagellum gene silencing in Examples 1-8.

### Example 11: Placement of CISP Along the Flagella Operon Disrupts Transcription

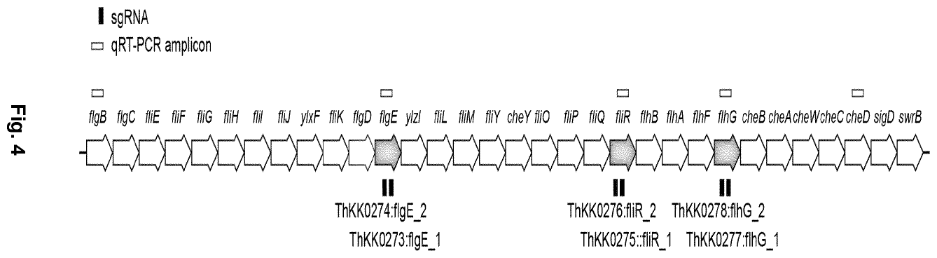

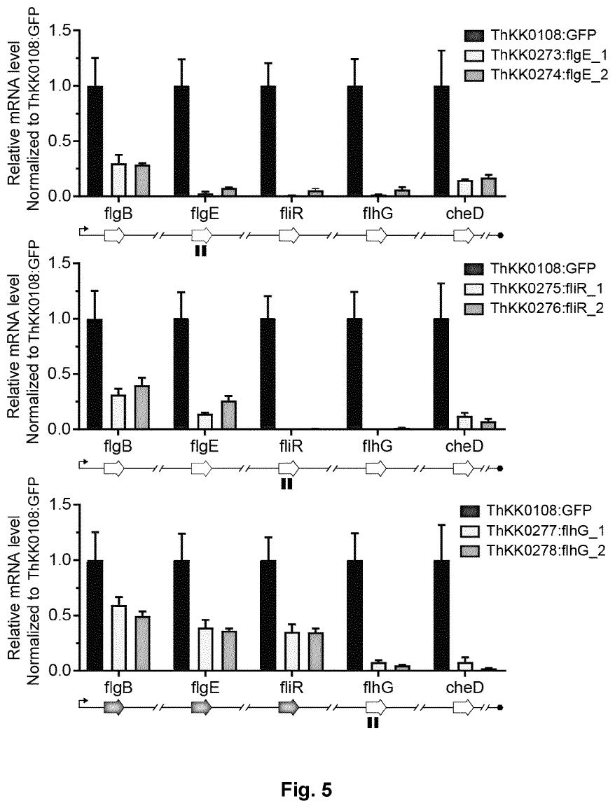

The effects on steady-state mRNA levels of the flagella operon when targeting CISP to it, was evaluated by qRT-PCR (FIG. 5). Exact positioning of sgRNA and qRT-PCR amplicons is shown in FIG. 4. As can be seen in FIG. 4, two sgRNAs were designed for each of the targets flgE, fliR, and flhG. We found that targeting said genes efficiently inhibits expression for each of the sgRNAs. With this approach, not only expression of the genes targeted by the sgRNAs was efficiently inhibited, but also other flagella genes in the operon downstream of the sgRNA target site, i.e. reduced expression of fliR, flhG and cheD after targeting of flgE, reduced expression of flhG and cheD after targeting of fliR, and reduced expression of cheD after targeting flhG (FIG. 5). In addition, the expression of genes upstream of the sgRNA was decreased to ˜30% compared to their expression levels in a strain expressing a sgRNA against gfp (FIG. 5).

### Example 12: Substitution of flgE With the ERM Plus a Terminator Disrupts Downstream Operon Transcription

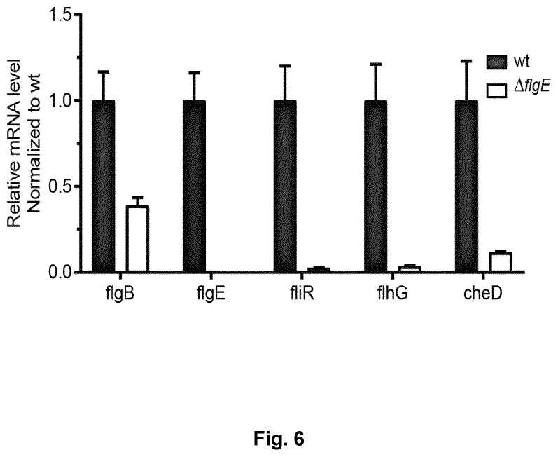

The steady-state mRNA levels of the flagella operon in BT11018, were evaluated by qRT-PCR (FIG. 6). Substituting the flgE gene with the erythromycin (Erm) selection marker followed by a hairpin terminator, abolished flgE expression as well as severely repressed expression of the downstream operon comprising fliR, flhG and cheD (FIG. 6). The expression of the transcript upstream of flgE, i.e. the transcript of flgB, was reduced to ˜40% of wild type levels.

### Example 13: Inserting Various Product Genes into flgE Deleted Strain

To evaluate the effect of the flgE deletion on different product genes, the synthetic constructs listed in Table 4 were ordered.

TABLE 4

| SEQ ID NO: | Construct name | Product (AA sequence) |
|---|---|---|
| 76 | Pcons-aprH-cat | Protease (SEQ ID |
| | | NO: 72) |
| 77 | Pcons-nattokinase-cat | Nattokinase (SEQ ID |
| | | NO: 73) |
| 78 | Pcons-xylanase-cat | Xylanase (SEQ ID |
| | | NO: 74) |
| 79 | P4199-Xanthan lyase-cat | Xanthan lyase (SEQ ID |
| | | NO: 75) |

The cat selection marker was included in all constructs. The synthetic constructs were then transformed into both AEB2718 (wt control) and BT11019 (flgE deletion strain) and selected on chloramphenicol, resulting in the following strains:
BT11103: B. subtilis AEB2718, amyE::Pcons-aprH-cat
BT11104: B. subtilis BT11019, amyE::Pcons-aprH-cat, flgE::ERM
BT11105: B. subtilis AEB2718, amyE::Pcons-Nattokinase-cat
BT11106: B. subtilis BT11019, amyE::Pcons-Nattokinase-cat, flgE::ERM
BT11109: B. subtilis AEB2718, amyE::Pcons-Xylanase-cat
BT11110: B. subtilis BT11019, amyE::Pcons-Xylanase-cat, flgE::ERM
BT11111: B. subtilis AEB2718, pel::P4199-Xathan lyase-cat
BT11112: B. subtilis BT11019, pel::P4199-Xathan lyase, flgE::ERM

### Example 14: The Deletion of flgE Incerases Productivity of a Range of Enzymes

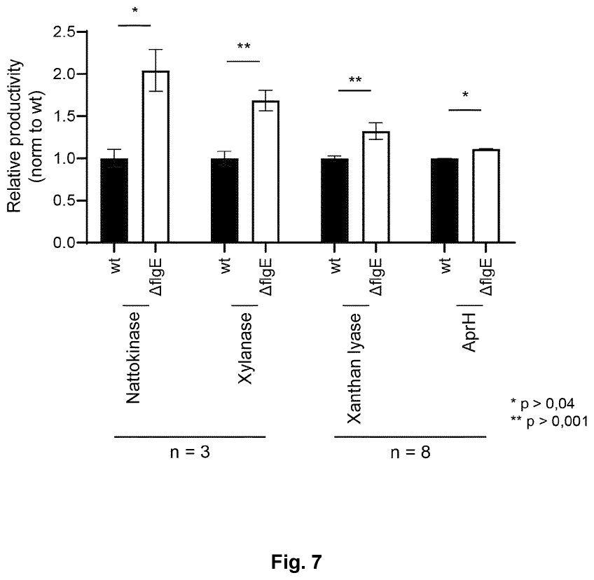

To evaluate the performance of strains BT11103, BT11104, BT11105. BT11106, BT11109, BT11110, BT11111, and BT11112, the strains were fermented in a Biolector and enzyme activity was measured (FIG. 7, error bars depict standard deviation). When the flgE deletion was normalized to the corresponding wild type, the following productivity improvements were observed: Nattokinase expression improved 2.04 fold, i.e. an increase of 104%, (+/−0.24 n=3, BT11106 normalized to BT11105), Xylanase expression improved 1.69 fold, i.e. an increase of 69%, (+/−0.12 n=3, BT11110 normalized to BT11109), Xanthan lyase expression improved 1.32 fold, i.e. an increase of 32%, (+/−0.10 n=8, BT11112 normalized to BT11111), and aprH (protease) expression improved 1.11 fold, i.e. an increase of 11%, (+/−0.004 n=8, BT11104 normalized to BT11103). The herein disclosed examples show that downregulation, disruption, and/or deletion of flagella genes increased productivity for different types of enzymes, e.g. amylase, nattokinase, xylanase, xanthan lyase, and protease.

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

The invention is further defined by the following numbered paragraphs:
1. A mutated bacterial host cell comprising a heterologous promoter operably linked to a first heterologous polynucleotide encoding one or more polypeptide of interest, wherein at least one flagellum gene is altered to encode an altered flagellum polypeptide; or wherein at least one flagellum gene is overexpressed, such as the MotI polypeptide.
2. The bacterial host cell of paragraph 1 wherein, wherein the altered flagellum polypeptide affects the flagella activity of said host cell by reducing or eliminating the flagella activity of the host cell.
3. The bacterial host cell according to any one of paragraphs 1 to 2, wherein the expression of the altered flagellum polypeptide is similar to the expression of the flagellum polypeptide in the non-mutated parent cell.
4. The bacterial host cell according to any one of paragraphs 1 to 3, wherein the altered flagellum gene comprises an alteration selected from a nucleotide deletion, nucleotide insertion and a premature stop codon.
5. The bacterial host cell according to paragraph 4, wherein the altered flagellum gene affects the functionality and/or activity of the flagella, wherein the flagella activity is reduced or eliminated.
6. A mutated bacterial host cell comprising a heterologous promoter operably linked to a first heterologous polynucleotide encoding one or more polypeptide of interest, wherein expression of at least one flagellum gene is reduced or eliminated compared to a non-mutated otherwise isogenic cell or parent cell.
7. The bacterial host cell according to any one of paragraphs 1 to 6, wherein the flagellum gene encodes a flagellum polypeptide selected from the list of FlgA, FlgB, FlgC, FlgD, FlgE, FlgF, FlgG, FlgH, FlgI, FlgJ, FlgK, FlgL, FlgM, FlgN, FlhA, FlhB, FlhC, FlhD, FlhE, FlhF, FlhG, FlhO, FlhP, FliA, FliD, FliE, FliF, FliG, FliH, FliI, FliJ, FliK, FliL, FliM, FliN, FliO, FliP, FliQ, FliR, FliS, FliT, FliY, FliZ, Hag, MotA, MotB, YlxF, SwrD, CheY, CheB, CheA, CheW, CheC, CheD, SigD, and SwrB; preferably the flagellum gene encodes the flagellum polypeptide FlgE, FliR or FlhG.
8. The bacterial host cell of any one of paragraphs 1 to 7, wherein the flagellum gene is selected from the list of flgA, flgB, flgC, flgD, flgE, flgF, flgG, flgH, flgI, flgJ, flgK, flgL, flgM, flgN, flhA, flhB, flhC, flhD, flhE, flhF, flhG, flhO, flhP, fliA, fliD, fliE, fliF, fliG, fliH, fliI, fliJ, fliK, fliL, fliM, fliN, fliO, fliP, fliQ, fliR, fliS, fliT, fliY, fliZ, hag, motA, motB, ylxF, swrD, cheY, cheB, cheA, cheW, cheC, cheD, sigD, and swrB; preferably the flagellum gene is flgE, fliR or flhG.
9. The bacterial host cell of any one of paragraphs 6 to 8, wherein the expression of one or more flagellum genes is reduced or eliminated, such as a reduced or eliminated expression of two, three, four, five, six, seven, eight, or more than eight flagella genes, independently chosen from one another.
10. The bacterial host cell of any one of paragraphs 6 to 9, wherein the expression of the flagellum gene flgE is reduced or eliminated in combination with a reduced or eliminated expression of the flagellum gene fliR.
11. The bacterial host cell of any one of paragraphs 6 to 10, wherein the expression of the flagellum gene flgE is reduced or eliminated in combination with a reduced or eliminated expression of the flagellum gene flhG.
12. The bacterial host cell of any one of paragraphs 6 to 11, wherein the expression of the flagellum gene flhG is reduced or eliminated in combination with a reduced or eliminated expression of the flagellum gene fliR.
13. The bacterial host cell of any one of paragraphs 6 to 12, wherein the expression of the flagellum gene flgE is reduced or eliminated in combination with a reduced or eliminated expression of the flagellum gene fliR, and with a reduced or eliminated expression of the flagellum gene flhG.
14. The bacterial host cell of any one of paragraphs 6 to 13, wherein the expression of the flagellum gene flgE, fliR and/or flhG is reduced or eliminated in combination with a reduced or eliminated expression of at least one flagellum gene selected from the list of flgA, flgB, flgC, flgD, flgF, flgG, flgH, flgI, flgJ, flgK, flgL, flgM, flgN, flhA, flhB, flhC, flhD, flhE, flhF, flhO, flhP, fliA, fliD, fliE, fliF, fliG, fliH, fliI, fliJ, fliK, fliL, fliM, fliN, fliO, fliP, fliQ, fliS, fliT, fliY, fliZ, hag, motA, motB, ylxF, swrD, cheY, cheB, cheA, cheVV, cheC, cheD, sigD, and swrB.
15. The bacterial host cell of any one of paragraphs 6 to 14, wherein the expression of two, three, four, or more than four flagella genes is reduced or eliminated, wherein the flagellum gene is selected from the list of flgA, flgB, flgC, flgD, flgE, flgF, flgG, flgH, flgI, flgJ, flgK, flgL, flgM, flgN, flhA, flhB, flhC, flhD, flhE, flhF, flhG, flhO, flhP, fliA, fliD, fliE, fliF, fliG, fliH, fliI, fliJ, fliK, fliL, fliM, fliN, fliO, fliP, fliQ, fliR, fliS, fliT, fliY, fliZ, hag, motA, motB, ylxF, swrD, cheY, cheB, cheA, cheW, cheC, cheD, sigD, and swrB.
16. The bacterial host cell of any one of paragraphs 1 to 15, wherein the flagellum gene comprises or consists of a polynucleotide sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to a polynucleotide sequence selected from the list of SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22 and SEQ ID NO: 23.
17. The bacterial host cell of any one of paragraphs 1 to 16, wherein the flagellum gene encodes a flagellum polypeptide comprising or consisting of an amino acid sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to a polypeptide sequence selected from the list of SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, and SEQ ID NO: 63.
18. The bacterial host cell of any one of paragraphs 1 to 17, wherein the polynucleotide sequence of the flagellum gene comprises at least one alteration selected from the list of a premature stop-codon, a nucleotide insertion, and a nucleotide deletion, such as the deletion of one or more nucleotides of the polynucleotide sequence of the flagellum gene or its coding region, or the deletion of substantially all nucleotides of the polynucleotide sequence of the flagellum gene or its coding region; or wherein the polynucleotide sequence of the flagellum gene or its coding region is deleted in its entirety.
19. The bacterial host cell of paragraph 18, wherein the flagellum gene is deleted by substituting the open reading frame of the flagellum gene with another polynucleotide sequence, such as a polynucleotide sequence encoding a selection marker.
20. The bacterial host cell of paragraph 19, wherein the flagellum gene is deleted by homologous recombination.
21. The bacterial host cell of any one of paragraphs 1 to 20, wherein the at least one alteration of the flagellum gene results in a flagellum gene polynucleotide sequence comprising or consisting of a polynucleotide sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100% to a polynucleotide sequence selected from the list of SEQ ID NO: 15, SEQ ID NO: 16 and SEQ ID NO: 17.
22. The bacterial host cell of any one of paragraphs 1 to 21, wherein the at least one alteration of the flagellum gene results in a flagellum polypeptide sequence comprising or consisting of a polypeptide sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100% to a polypeptide sequence selected from the list of SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, and SEQ ID NO: 63.
23. The bacterial host cell of any one of paragraphs 1 to 22, wherein the host cell comprises a reduced or eliminated flagella activity, a reduced or eliminated cell motility, or a reduced or eliminated swimming activity.
24. The bacterial host cell of paragraph 23, wherein the cell motility of the mutated host cell is 35 reduced by at least 10%, e.g., at least 15%, at least 20%, at least 30%, at least 40%, at least 50%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or by 100% compared to a non-mutated otherwise isogenic or parent cell.
25. The bacterial host cell of any one of paragraphs 1 to 24, wherein the host cell is a Gram-negative bacteria selected from the group consisting of Campylobacter, E. coli, Flavobacterium, Fusobacterium, Helicobacter, Ilyobacter, Neisseria, Pseudomonas, Salmonella, and Ureaplasma cells, or wherein the host cell is a Gram-positive cell selected from the group consisting of Bacillus, Clostridium, Enterococcus, Geobacillus, Lactobacillus, Lactococcus, Oceanobacillus, Staphylococcus, Streptococcus, or Streptomyces cells, such as Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, Bacillus thuringiensis, Streptococcus equisimilis, Streptococcus pyogenes, Streptococcus uberis, and Streptococcus equi subsp. Zooepidemicus, Streptomyces achromogenes, Streptomyces avermitilis, Streptomyces coelicolor, Streptomyces griseus, and Streptomyces lividans cells, preferably the host cell is a Bacillus cell, most preferably a Bacillus subtilis or a Bacillus licheniformis cell.
26. The bacterial host cell of any one of paragraphs 1 to 24, wherein the host cell is a Bacillus subtilis cell.
27. The bacterial host cell of any one of paragraphs 1 to 24, wherein the host cell is a Bacillus licheniformis cell.
28. The bacterial host cell of any one of paragraphs 1 to 27, wherein the polypeptide of interest comprises an enzyme; preferably the enzyme is selected from the group consisting of hydrolase, isomerase, ligase, lyase, oxidoreductase, or transferase; more preferably an aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, alpha-glucosidase, beta-glucosidase, invertase, laccase, lipase, mannosidase, mutanase, nuclease, oxidase, pectinolytic enzyme, peroxidase, phosphodiesterase, phytase, polyphenoloxidase, proteolytic enzyme, ribonuclease, transglutaminase, xylanase, and beta-xylosidase; even more preferably the polypeptide of interest comprises an amylase.
29. The bacterial host cell of any one of paragraphs 1 to 28, wherein the polypeptide of interest comprises an amylase, such as an amylase which comprises, consists essentially of, or consists of the mature polypeptide having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the amino acid sequence of SEQ ID NO: 8.
30. The bacterial host cell of any one of paragraphs 1 to 28, wherein the polypeptide of interest comprises, consists of, or essentially consists of a protease.
31. The bacterial host cell according to any one of paragraphs 1 to 28, wherein the one or more polypeptide of interest comprises a protease, such as a protease which comprises, consists essentially of, or consists of the mature polypeptide having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the amino acid sequence of SEQ ID NO: 72.
32. The bacterial host cell according to any one of paragraphs 1 to 28, wherein the one or more polypeptide of interest comprises a nattokinase, such as a nattokinase which comprises, consists essentially of, or consists of the mature polypeptide having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the amino acid sequence of SEQ ID NO: 73.
33. The bacterial host cell according to any one of paragraphs 1 to 28, wherein the one or more polypeptide of interest comprises a xylanase, such as a xylanase which comprises, consists essentially of, or consists of the mature polypeptide having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the amino acid sequence of SEQ ID NO: 74.
34. The bacterial host cell according to any one of paragraphs 1 to 28, wherein the one or more polypeptide of interest comprises a xanthan lyase, such as a xanthan lyase which comprises, consists essentially of, or consists of the mature polypeptide having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the amino acid sequence of SEQ ID NO: 75.
35. The bacterial host cell according to any one of paragraphs 1 to 28, wherein the one or more polypeptide of interest comprises a xanthanase.
36. The bacterial host cell of any one of paragraphs 1 to 28, wherein the polypeptide of interest comprises, consists of, or essentially consists of a phosphodiesterase (PDE).
37. The bacterial host cell of any one of paragraphs 1 to 36, wherein the polypeptide is heterologous to the recombinant host cell.
38. The bacterial host cell of any one of paragraphs 1 to 37, wherein at least one of the one or more control sequences is heterologous to the polynucleotide encoding the polypeptide.
39. The bacterial host cell of any one of paragraphs 1 to 38, wherein the host cell comprises at least two copies, e.g., three, four, or five, of the polynucleotide encoding the polypeptide of interest.
40. The bacterial host cell of any one of paragraphs 1 to 39, wherein the host cell further comprises a heterologous promoter operably linked to a second heterologous polynucleotide encoding a non-coding RNA molecule, said non-coding RNA molecule being configured to form a RNA:DNA heteroduplex with a target polynucleotide sequence of a flagellum gene by hybridizing or binding with/to said target polynucleotide sequence of the flagellum gene, or being configured to form a RNA:RNA heteroduplex with a target polynucleotide sequence of a RNA transcript of the flagellum gene by hybridizing or binding with/to said target polynucleotide sequence of the RNA transcript.
41. The host cell of paragraph 40, wherein the second heterologous polynucleotide encodes a non-coding RNA molecule comprising, essentially consisting of or consisting of a polynucleotide sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the polynucleotide sequence of SEQ ID NO: 1, SEQ ID NO: 2, SEQ ID NO: 3, SEQ ID NO: 4, SEQ ID NO: 5 or SEQ ID NO: 6.
42. The bacterial host cell of any one of paragraphs 40 to 41, wherein the second heterologous polynucleotide consists of, essentially consists of, or comprises a single guide RNA.
43. The bacterial host cell of any one of paragraphs 40 to 42, wherein the host cell comprises a third heterologous polynucleotide encoding a catalytically inactive site-directed polypeptide (CISP), the CISP being selected from the group consisting of a catalytically inactive CRISPR-associated protein, a catalytically inactive zinc finger nuclease (ZFN), a zinc finger, a catalytically inactive transcription activator-like effector nuclease (TALEN), a TALE, a catalytically inactive meganuclease, and a catalytically inactive MAD7/Cas12a endonuclease.
44. The bacterial host cell of paragraph 43, wherein the CISP is configured to bind to a binding sequence of:
   i) the polynucleotide sequence of the non-coding RNA molecule or ncRNA, OR
   ii) the DNA or RNA polynucleotide sequence of the RNA:DNA heteroduplex, OR
   iii) a RNA polynucleotide sequence of the RNA:RNA heteroduplex, such as the ncRNA of the RNA:RNA heteroduplex.
45. The bacterial host cell of any one of paragraphs 43 to 44, wherein the CISP is configured to bind to the binding sequence of the polynucleotide sequence of the non-coding RNA molecule, such as a ncRNA molecule.
46. The bacterial host cell of any one of paragraphs 43 to 45, wherein the CISP is configured to bind to a guide RNA molecule.
47. A method of producing a mutant of a parent cell, comprising inactivating at least one polynucleotide encoding a flagellum polypeptide, which results in the mutant producing less of the flagellum polypeptide than the parent cell.
48. A mutant cell produced by the method of producing a mutant of a parent cell of paragraph 47.
49. The mutant cell of paragraph 48 further comprising a gene encoding a native or heterologous protein.
50. A double-stranded inhibitory RNA (dsRNA) molecule comprising a subsequence of a polynucleotide sequence of a flagellum gene, wherein optionally the dsRNA is an siRNA or a miRNA molecule.
51. The dsRNA molecule of paragraph 50, wherein the double-stranded inhibitory RNA (dsRNA) molecule is about 15, 16, 17, 18, 19, 20, 21, 22, 23, 24, 25 or more duplex nucleotides in length.
52. A method of inhibiting the expression of a flagellum polypeptide in a cell, comprising administering to the cell or expressing in the cell said double-stranded inhibitory RNA (dsRNA) molecule of any one of paragraphs 50 to 51.
53. A mutant cell produced by the method of inhibiting the expression of a flagella polypeptide of paragraph 52.
54. A method for producing one or more polypeptide of interest, the method comprising:
   i) providing a bacterial host cell according to any one of paragraphs 1 to 49, or according to paragraph 53,
   ii) cultivating said host cell under conditions conducive for expression of the one or more polypeptide of interest; and,
   iii) optionally recovering the polypeptide of interest.
55. A nucleic acid construct comprising at least one polynucleotide encoding a flagellum polypeptide comprising or consisting of a polypeptide sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, but less than 100% to any one of SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, and SEQ ID NO: 63.
56. A nucleic acid construct comprising a heterologous promoter operably linked to a polynucleotide encoding a non-coding RNA molecule, wherein the non-coding RNA molecule is configured to form a RNA:DNA heteroduplex with a target polynucleotide sequence of a bacterial flagellum gene by hybridizing with said target polynucleotide sequence of the bacterial flagellum gene, or configured to form a RNA:RNA heteroduplex with a target polynucleotide sequence of a RNA transcript of the flagellum gene by hybridizing with said target polynucleotide sequence of the RNA transcript.
57. The nucleic acid construct according to paragraph 56, wherein the non-coding RNA molecule comprises, essentially consists of, or consists of a polynucleotide sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the polynucleotide sequence of (i) the polynucleotide sequence of SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22 or SEQ ID NO: 23; (ii) the transcript of SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22 or SEQ ID NO: 23; or (iii) the complementary polynucleotide sequence of SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22 or SEQ ID NO: 23.
58. The nucleic acid construct according to any one of paragraphs 56 to 57, wherein the non-coding RNA molecule comprises, essentially consists of, or consists of a polynucleotide sequence having a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% to the polynucleotide sequence of SEQ ID NO: 1, SEQ ID NO: 2, SEQ ID NO: 3, SEQ ID NO: 4, SEQ ID NO: 5 or SEQ ID NO: 6.
59. The nucleic acid construct according to any one of paragraphs 56 to 58, comprising a polynucleotide sequence encoding a LISP.
60. The nucleic acid construct according to any one of paragraphs 55 to 59, wherein a promoter is operably linked to any one of the polynucleotides encoding the mutated flagellum polypeptide, the non-coding RNA or the LISP, such as a homologous or heterologous promoter, preferably a heterologous promoter.
61. An expression vector comprising a nucleic acid construct according to any one of paragraphs 55 to 60.
62. A recombinant host cell comprising the polynucleotide according to any one of paragraphs 55 to 60, or the expression vector of paragraph 61.
63. A method of producing a mutant of a parent cell, comprising inactivating one or more polynucleotide encoding a flagellum polypeptide, which results in the mutant producing less of the flagellum polypeptide than the parent cell, preferably the polynucleotide is inactivated by gene silencing, gene deletion, nucleotide insertion, nucleotide deletion or a premature stop-codon.
64. The method of paragraph 63, wherein the polynucleotide is a flagellum gene selected from the list of flgA, flgB, flgC, flgD, flgE, flgF, flgG, flgH, flgI, flgJ, flgK, flgL, flgM, flgN, flhA, flhB, flhC, flhD, flhE, flhF, flhG, flhO, flhP, fliA, fliD, fliE, fliF, fliG, fliH, fliI, fliJ, fliK, fliL, fliM, fliN, fliO, fliP, fliQ, fliR, fliS, fliT, fliY, fliZ, hag, motA, motB, ylxF, swrD, cheY, cheB, cheA, cheW, cheC, cheD, sigD, and swrB.
65. A mutant cell produced by the method of paragraph 63 or 64. 66. The mutant cell of paragraph 65, further comprising a gene encoding a native or heterologous protein.
67. A method of producing a protein, comprising cultivating the mutant cell of paragraph 65 or 66 under conditions conducive for production of the protein.
68. The method of paragraph 67, further comprising recovering the protein.

1-15. (canceled)

16. A mutated bacterial host cell comprising a heterologous promoter operably linked to a first heterologous polynucleotide encoding one or more polypeptides of interest, wherein expression of at least one flagellum gene is reduced or eliminated compared to a non-mutated otherwise isogenic cell or parent cell.

17. The bacterial host cell according to claim 16, wherein the at least one flagellum gene encodes a flagellum polypeptide selected from FlgA, FlgB, FlgC, FlgD, FlgE, FlgF, FlgG, FlgH, FlgI, FlgJ, FlgK, FlgL, FlgM, FlgN, FlhA, FlhB, FlhC, FlhD, FlhE, FlhF, FlhG, FlhO, FlhP, FliA, FliD, FliE, FliF, FliG, FliH, FliI, FliJ, FliK, FliL, FliM, FliN, FliO, FliP, FliQ, FliR, FliS, FliT, FliY, FliZ, Hag, MotA, MotB, YlxF, SwrD, CheY, CheB, CheA, CheW, CheC, CheD, SigD, and SwrB.

18. The bacterial host cell according to claim 16, wherein the at least one flagellum gene encodes the flagellum polypeptide FlgE, FliR and/or FlhG.

19. The bacterial host cell according to 16, wherein the at least one flagellum gene encodes one or more flagellum polypeptides comprising or consisting of an amino acid sequence having a sequence identity of at least 80% to a polypeptide sequence selected from SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, and SEQ ID NO: 63.

20. The bacterial host cell according to claim 16, wherein the at least one flagellum gene encodes one or more flagellum polypeptides comprising or consisting of an amino acid sequence selected from SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, and SEQ ID NO: 63.

21. The bacterial host cell according to claim 16, wherein the polynucleotide sequence of the at least one flagellum gene comprises at least one alteration selected from the list of a premature stop-codon, a nucleotide insertion, and a nucleotide deletion, such as the deletion of one or more nucleotides of the at least one polynucleotide sequence of the flagellum gene or its coding region, or the deletion of substantially all nucleotides of the polynucleotide sequence of the at least one flagellum gene or its coding region; OR wherein the polynucleotide sequence of the at least one flagellum gene or its coding region is fully deleted.

22. The bacterial host cell according to claim 16, wherein the host cell comprises a reduced or eliminated flagellum activity, a reduced or eliminated cell motility, or a reduced or eliminated swimming activity.

23. The bacterial host cell according to claim 16, wherein the host cell is a Bacillus cell.

24. The bacterial host cell according to claim 16, wherein the host cell is a Bacillus subtilis or a Bacillus licheniformis cell.

25. The bacterial host cell according to claim 16, wherein the one or more polypeptides of interest comprises an enzyme.

26. The bacterial host cell according to claim 16, wherein the one or more polypeptides of interest comprises an amylase, a protease, a nattokinase, a xylanase, and/or a xanthan lyase.

27. The bacterial host cell according to claim 16, wherein the one or more polypeptides of interest comprises an amylase which comprises or consists of a mature polypeptide having a sequence identity at least 80% to the amino acid sequence of SEQ ID NO: 8.

28. The bacterial host cell according to claim 16, wherein the one or more polypeptides of interest comprises an amylase which comprises or consists of a mature polypeptide having the amino acid sequence of SEQ ID NO: 8.

29. The bacterial host cell according to claim 16, wherein the one or more polypeptides of interest comprises an amylase which comprises or consists of a mature polypeptide having a sequence identity at least 80% to the amino acid sequence of SEQ ID NO: 8.

30. The bacterial host cell according to claim 16, wherein the one or more polypeptides of interest comprises an amylase which comprises or consists of a mature polypeptide having the amino acid sequence of SEQ ID NO: 8.

31. The bacterial host cell according to claim 16, wherein the one or more polypeptides of interest comprises a nattokinase which comprises or consists of a mature polypeptide having a sequence identity at least 80% to the amino acid sequence of SEQ ID NO: 73.

32. The bacterial host cell according to claim 16, wherein the one or more polypeptides of interest comprises a nattokinase which comprises or consists of a mature polypeptide having the amino acid sequence of SEQ ID NO: 73.

33. The bacterial host cell according to claim 16, wherein the one or more polypeptides of interest comprises a xylanase which comprises or consists of a mature polypeptide having a sequence identity at least 80% to the amino acid sequence of SEQ ID NO: 74.

34. The bacterial host cell according to claim 16, wherein the one or more polypeptides of interest comprises a xylanase which comprises or consists of a mature polypeptide having the amino acid sequence of SEQ ID NO: 74.

35. The bacterial host cell according to claim 16, wherein the one or more polypeptides of interest comprises a xanthan lyase which comprises or consists of a mature polypeptide having a sequence identity at least 80% to the amino acid sequence of SEQ ID NO: 75.

36. The bacterial host cell according to claim 16, wherein the one or more polypeptides of interest comprises a xanthan lyase which comprises or consists of a mature polypeptide having the amino acid sequence of SEQ ID NO: 75.

37. A method for producing one or more polypeptides of interest, the method comprising:
i) providing a bacterial host cell according to claim 16,
ii) cultivating said host cell under conditions conducive for expression of the one or more polypeptide of interest; and,
iii) optionally recovering the one or more polypeptides of interest.

38. A nucleic acid construct comprising a polynucleotide encoding at least one flagellum polypeptide comprising or consisting of a polypeptide sequence having a sequence identity of at least 80% but less than 100% to any one of SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, and SEQ ID NO: 63.

39. An expression vector comprising a nucleic acid construct according to claim 38.
